(19) United States
(12) Patent Application Publication
(10) Pub. No.: US 2023/0346902 A1
(43) Pub. Date: Nov. 2, 2023

(54) Shigella-Tetravalent (Shigella4V) Bioconjugate

(71) Applicant: GlaxoSmithKline Biologicals SA, Rixensart (BE)

(72) Inventors: Martin Edward BRAUN, Rixensart (BE); Rainer FOLLADOR, Rixensart (BE); Stefan Jochen KEMMLER, Rixensart (BE)

(73) Assignee: GlaxoSmithKline Biologicals SA, Rixensart (BE)

(21) Appl. No.: 18/001,551

(22) PCT Filed: Jun. 17, 2021
(86) PCT No.: PCT/IB2021/055361

§ 371 Date: Dec. 12, 2022

Related U.S. Application Data
(60) Provisional application No. 63/043,883, filed on Jun. 25, 2020.
(60) Provisional application No. 63/040,844, filed on Jun. 18, 2020.

(30) Foreign Application Priority Data
Jun. 25, 2020 (EP) 20182138.6
Jun. 25, 2020 (EP) 20182139.4

(51) Int. Cl. A61K 39/112; A61P 31/04

(52) U.S. Cl. CPC A61K 39/0283; A61P 31/04; A61K 2039/6037

(57) ABSTRACT

A composition comprising Shigella-Tetravalent (4-valent Shigella) bioconjugates. That encompasses the Shigella O-polysaccharide antigens of serotypes Shigella flexneri 2a, 3a, 6 and Shigella sonnei covalently linked to the protein carrier.

### SEQUENCE LISTING

The instant application contains a Sequence Listing which has been submitted electronically in ASCII format and is hereby incorporated by reference in its entirety. Said ASCII copy, created on May 13, 2021, is named VU66948_SL.txt and is 81,530 bytes in size.

### FIELD OF THE INVENTION

The present invention relates to a composition comprising Shigella-Tetravalent (4-valent Shigella) bioconjugates. More particularly, the invention encompasses the Shigella O-polysaccharide antigens of serotypes Shigella flexneri 2a, 3a, 6 and Shigella sonnei covalently linked to the protein carrier.

The four bioconjugates may be produced separately by a process starting with cell substrates. In common to all four cell substrates is the same type of original host, the replacement of a polysaccharide biosynthesis (rfb) cluster by an O-polysaccharide cluster, introduction of a plasmid encoding a carrier protein, and a plasmid encoding the oligosaccharyltransferase PglB or PglL.

### BACKGROUND OF THE INVENTION

Diarrhea disease (DD) is a disease of major importance for children/infants in low- and middle-income countries (LMICs). According to the Global Burden of Disease 2015, diarrhea is the fourth leading cause of death for children and is responsible for 8.6% of all deaths in children aged under five years old. Despite the medical need for a vaccine against Shigella in children in LMICs, no vaccine is currently on the market.

Shigella is the leading pathogen causing DD in LMICs in children above 1 year of age and is among the top six pathogens causing diarrhea in children below 1 year of age (1-3). About 11% of diarrhea deaths are due to Shigella, leading to 55,900 to 65,000 deaths per year, mostly children. Shigella is a gram negative non-sporulating, facultative anaerobe and primate-restricted pathogen. Transmission can occur via the fecal-oral route, through contaminated water, fomites, food or direct contact. A low bacterial load (between 100 and 1000 colony forming units) can result in a symptomatic infection.

Following an incubation time between 1 and 4 days, shigellosis is typically diagnosed by fever, watery diarrhea, enteric symptoms like anorexia, abdominal cramps and vomiting, and dysentery (acute colitis of distal colon/rectum with blood in stools). Such clinical manifestations are normally self-limited in immunocompetent adults whereas in children below 5 years of age they can result in intestinal or metabolic complications, with toxic megacolon and hemolytic uremic syndrome being the major complications (i.e., intestinal perforation, rectal prolapse or hypoglycemia, hyponatremia, dehydration).

Clinical outcomes associated to the acute phase of Shigella infection, especially if repeated and prolonged, are also responsible for post-infection complications, such as reactive arthritis and irritable bowel syndrome, as well as developmental complications with reduction in cognitive performance and a drop of height-for-age Z (HAZ) score. Indeed, the unseen impact of DD has long term consequences for children and households, with a high burden driving to further impoverishment at household level and lost schooling translating into reduced potential earnings.

Current disease burden estimates may still under-represent the incidence of DD, which is affected by various methodologies used in different studies as well as the sensitivity of the diagnostic techniques used. Shigella is conventionally diagnosed by isolation on stool culture, subsequent standard biochemical testing, and serotyping via serum agglutination. Isolating colonies of the pathogens from stool is time consuming and difficult, especially if antibiotics were used or if bacteria remain in non-dividing states when cultured. Such challenges reported by conventional culture diagnostic methods have highlighted the need for new methods non-culture-dependent (like quantitative PCR). Indeed, results from the re-analysis of samples subsets from GEMS (Global Enteric Multicenter Study) and MAL-ED (Etiology, Risk Factors, and Interactions of Enteric Infections and Malnutrition and the Consequences for Child Health and Development) studies in pediatric populations, have indicated that prior studies of Shigella-attributable diarrhea in children may have underestimated the true incidence by at least two-fold.

The general clinical management of the disease includes rehydration (oral or intravenous), zinc supplementation and antibiotic treatment. However, resistance to traditional first-and second line antibiotics such as ampicillin/ trimethoprim-sulfamethoxazole or ciprofloxacin/azithromycin has been increasingly reported. Consequently, because initial treatment can fail, resistant infections can last longer than infections with susceptible bacteria, with consequently more severe clinical outcomes and higher costs for the health-care system.

Prevention of DD caused by Shigella, with the introduction of an efficacious and affordable vaccine, is especially important in LMICs, where other interventions, i.e. health care access, safe water, sanitation, and hygiene are difficult to be achieved in the short term.

Immunity to Shigella appears to be strain-specific. Four species belong to the Shigella genus; flexneri, sonnei, boydii and dysenteriae, with respectively 19, 20, 1 and 15 serotypes (different O antigen structures) each. Important for vaccine development considerations is the distribution of Shigella serotypes. The predominant serotypes responsible for moderate-to-severe disease in children of the developing world are Shigella flexneri 2a, S. sonnei and S. flexneri types 3a and 6. [1] [2] In particular, the long-term-trends of global Shigella species distribution shows an increase in S. sonnei in regions that have undergone significant industrialization compared to rural areas where S. flexneri levels remain high and with a very heterogeneous geographic distribution.

There remains an unmet need for the provision of a vaccine against shigella. There also remains difficulty in synthesizing bioconjugates with particular reducing end sugars.

### SUMMARY OF THE INVENTION

A multivalent Shigella conjugate using bioconjugation technology has been developed. The vaccine candidate, Shigella-Tetravalent (Shigella4V (S-4V)), is an immunogenic composition composed of O-antigen polysaccharides (PS) bioconjugates from four different Shigella strains representing the most epidemiologically relevant strains: S. sonnei (SsE), S. flexneri 2a, 3a and 6 (Sf2E, Sf3E, Sf6E). In an embodiment, each PS is conjugated to the recombinant Pseudomonas aeruginosa Exoprotein A, rEPA. The candidate encompasses the Shigella O-polysaccharide antigens of serotypes Shigella flexneri 2a, 3a, 6 and Shigella sonnei covalently linked to the protein carrier.

For example, for Sf2E, Sf3E and Sf6E the polysaccharide is linked covalently via the reducing end of the O-antigen to the side chain nitrogen atom of an asparagine residue (4) residing in a consensus sequence for N- glycosylation (see Table 3). The signal peptide (underlined letters) is cleaved off during translocation to the periplasm. The N-glycosylation consensus sites are marked with bold letters. The Leu-Glu to Val mutation (italicized) leads to a significant detoxification of EPA.

For SsE, the polysaccharide is linked covalently via the reducing end of the O-antigen to the side chain oxygen atom of a serine residue residing in the O-glycosylation site (see Table 4). The signal peptide (underlined letters) is cleaved off during translocation to the periplasm. The O-glycosylation consensus site is bold with the putative O-glycosylated Serine in underlined. The Leu-Glu to Val mutation (italicized) leads to a significant detoxification of EPA.

Bioconjugation technology is a versatile tool to deliver safe, well defined and immunogenic glycoconjugate vaccines at high yields. For O-antigen based vaccines the technology is particularly well suited due to the use of a common biosynthetic pathway. Bioconjugation enables the biosynthesis of conjugate vaccines with complex polysaccharide structures in engineered E. coli. Bioconjugates are immunogenic complexes of polysaccharides and proteins that are directly synthesized in vivo using appropriately engineered bacterial cells.

The identification of N-linked glycoproteins in Campylobacter jejuni demonstrated that prokaryotes can N-glycosylate their proteins. A specific Campylobacter enzyme (the oligosaccharyltransferase PgIB) is able to transfer an oligosaccharide from a lipid-linked carrier to the side chain of the amino acid asparagine when located in a particular consensus sequence within the polypeptide chain of the protein carrier. [19] This protein glycosylation system has been functionally transferred into Escherichia coli, enabling the production of glycoproteins in a well characterized and frequently used bacterial expression host. Using recombinant DNA technologies, this glycosylation machinery can be modified to produce various polysaccharides, which can be transferred to different acceptor proteins. Corresponding glycoengineering strategies for the production of novel bioconjugates are developed, allowing the production of bioconjugates that can be used as novel vaccines.

In summary, in the bioconjugation process, PglB as well as similar oligosaccharyltransferases more recently discovered [20], i.e. PglL, act to transfer diverse polysaccharides to a protein carrier (e.g. exotoxin A of Pseudomonas aeruginosa, EPA) present in the periplasm of E. coli, from which the resulting bioconjugate is subsequently harvested using periplasmic extraction and subsequent purification, FIG. 1A and FIG. 1B. [21]

The advantages of the bioconjugation technology include: (1) better immunogenicity of the conjugated antigens (PS and protein) due to absence of modifications by the conjugation chemistry and due to the configuration of PS antigen, (2) bioconjugates quality is reproducible: Bioconjugates are characterized for detailed structure at the drug substance level, and any quality issue is detected by high resolution technologies, (3) bioconjugates do not cause competing, chemical linker derived immune reactions (there are no chemical linkers) (Immune reaction to chemical conjugates often suffer from anti cross linker responses that prevail antigen specific responses), (4) simplified manufacturing result in reproducible-quality product, the bioconjugate is produced entirely in recombinant non-pathogenic E. coli and no growth of pathogenic organisms for the extraction of antigenic polysaccharides is required, resulting in advantages for the safety of the product and manufacturing, (5) the defined structure enables detailed analytical testing, both on drug substance and drug product level, as well as freezing of the product (if required), (6) no free polysaccharides and only minor amount of product related impurities are present after manufacturing, the enzymatic in vivo conjugation does not denature the protein carrier, therefore conserving important B-cell epitopes and correct protein folding opening the possibility to develop a bioconjugate that contains protein as well as sugar epitopes from the same organism, potentially broadening the protection of the respective vaccine, since no chemical treatments such as removal of endotoxin and crosslinking are necessary and the length of the polysaccharide is controlled in vivo, (the conjugates contain a defined and reproducible sugar pattern), and (7) the bioconjugation technology may enable the production of antigens that cannot be produced with existing technologies due to the chemical lability of the antigenic polysaccharide.

### DESCRIPTION OF THE FIGURES

FIG. 1A: In vivo bioconjugation process using recombinant Escherichia coli: polysaccharide (PS) and carrier protein genes are transferred to E. coli. Engineered bacteria are fermented. During fermentation, PS chains and carrier protein are produced and conjugated. Once fermentation is complete, conjugated proteins are purified from the bacterial periplasm.

FIG. 1B: Conjugation process: Campylobacter oligosaccharyltransferase (PglB) transfers polysaccharides from a lipid carrier to the Pseudomonas aeruginosa exoprotein A (EPA) protein carrier in the periplasm.

FIG. 2: Detailed schematic representation of the in-vivo protein glycosylation process.

FIG. 3: Structural properties of the S. flexneri 2a -antigen polysaccharide (PS). The reducing end and biological starting point of synthesis is GlcNAc. L-Rha: L-Rhamnose, Rha; D-Glc: D-Glucose, Glc; D-GlcNAc: D-N-acetyl-glucosamine, GlcNAc.

FIG. 4: Structural properties of the S. flexneri 3a -antigen polysaccharide (PS). The reducing end and biological starting point of synthesis is GlcNAc. L-Rha: L-Rhamnose, Rha; D-Glc: D-Glucose, Glc; D-GlcNAc: D-N-acetyl-glucosamine, GlcNAc.

FIG. 5: Structural properties of the S. flexneri 6 -antigen polysaccharide (PS). The reducing end and biological starting point of synthesis is GalNAc. L-Rha: L-Rhamnose, Rha; D-GalA: D-Galacturonic acid, GalA; D-GalNAc: D-N-acetylgalactosamine, GalNAc.

FIG. 6: Structural properties of the S. sonnei -antigen polysaccharide (PS). The reducing end and biological starting point of synthesis is D-FucNAc4N. D-FucNAc4N: 2-acetamido-4-amino-2, 4-dideoxy-D-fucose, FucNAc4N; LAltNAcA: 2-acetamido-2-deoxy-L-altruronic acid, AltNAcA.

FIG. 7: Degree of glycosylation characterization by SDS-PAGE of the Sf2E ENG and GMP API batches.

FIG. 8: Degree of glycosylation characterization by SDS-PAGE of the Sf3E ENG and GMP API batches.

FIG. 9: Degree of glycosylation characterization by SDS-PAGE of the Sf6E ENG and GMP API batches.

FIG. 10: Monosaccharide composition analysis by HPAEC-PAD of Sf2E GMP API batch.

FIG. 11: Monosaccharide composition analysis by HPAEC-PAD of Sf3E GMP API batch.

FIG. 12: Monosaccharide composition analysis by HPAEC-PAD of Sf6E GMP API batch.

FIG. 13: Monosaccharide composition analysis by HPAEC-PAD of Sf6E GMP API batch, zoomed overlay.

FIG. 14: Glycan structure characterization of SsE ENG API batch by hydrazinolysis. *FucNAc4N is deacetylated during hydrazinolysis and amino groups in position 2 and 4 are both re-acetylated during process.

FIG. 15: Glycan structure characterization of SsE GMP API batch by hydrazinolysis. *FucNAc4N is deacetylated during hydrazinolysis and amino groups in position 2 and 4 are both re-acetylated during process.

FIG. 16: Glycan structure characterization of the Sf2E ENG API batch by hydrazinolysis.

FIG. 17: Glycan structure characterization of the Sf2E GMP API batch by hydrazinolysis.

FIG. 18: Glycan structure characterization of the Sf3E ENG API batch by hydrazinolysis.

FIG. 19: Glycan structure characterization of the Sf3E GMP API batch by hydrazinolysis.

FIG. 20: Glycan structure characterization of the Sf6 ENG API batch by hydrazinolysis.

FIG. 21: Glycan structure characterization of the Sf6E GMP API batch by hydrazinolysis.

FIG. 22A: 1H-NMR spectra of SsE recorded at 600 MHz (313 K). The full 1H-NMR spectrum. Diagnostic anomeric and ring signals are labelled. Small peaks are from the terminal RU.

FIG. 22B: 1H-NMR spectra of SsE recorded at 600 MHz (313 K). 1D DOSY expansion of the anomeric region and ring regions. Diagnostic anomeric and ring signals are labelled. Small peaks are from the terminal RU.

FIG. 23: Left panel: The 2D 1H-13C overlay for SsE HSQC/ HMBC recorded at 600 MHz (313 K); the HMBC was optimized for J = 6 Hz. Proton/carbon crosspeaks have been labelled according to the corresponding residue (A= AltNAcA and B= FucNAcN). The major crosspeaks are labelled and the key inter-residue correlations are in squares. Small crosspeaks are from the terminal RU (tRU). Right panel: Expansion of the HSQC spectrum of SsE recorded at 600 MHz (313 K), the crosspeaks from the methyl region of the spectrum are shown in the inset. Key disaccharide repeating unit proton/carbon crosspeaks have been labelled as well as the small crosspeaks from the terminal AltNAcA.

FIG. 24: Stability of O-Acetyl-groups of Shigella4V IMP formulated in different buffers. Samples were stored at +37° C. for four weeks. The concentration of free, total and bound acetate as determined by ion chromatography is shown for the different formulations. An increase in free acetate, combined with a reduction in bound acetate is indicative for a loss of O-acetyl groups. Formulations are based on 10 mM sodium phosphate, 150 mM and S4V DP-18: pH 6.5 w/o Polysorbate 80, S4V DP-19: pH 6.5 0.015% Polysorbate 80, S4V DP-20: pH 7.0 w/o Polysorbate 80 and S4V DP-21: pH 7.0 0.015% Polysorbate 80.

FIG. 25A: Sf2a-LPS, Sf3a-LPS, Sf6-LPS, Ss-LPS and EPA-specific serum IgG titers in pre- and post-immunization rabbit sera by treatment group. Lines indicate the GMT +/- 95% confidence interval.

FIG. 25B: Sf2a-LPS, Sf3a-LPS, Sf6-LPS, Ss-LPS and EPA-specific serum IgG titers in pre- and post-immunization rabbit sera by treatment group. Lines indicate the GMT +/- 95% confidence interval.

FIG. 25C: Sf2a-LPS, Sf3a-LPS, Sf6-LPS, Ss-LPS and EPA-specific serum IgG titers in pre- and post-immunization rabbit sera by treatment group. Lines indicate the GMT +/- 95% confidence interval.

FIG. 25D: Sf2a-LPS, Sf3a-LPS, Sf6-LPS, Ss-LPS and EPA-specific serum IgG titers in pre- and post-immunization rabbit sera by treatment group. Lines indicate the GMT +/- 95% confidence interval.

FIG. 25E: Sf2a-LPS, Sf3a-LPS, Sf6-LPS, Ss-LPS and EPA-specific serum IgG titers in pre- and post-immunization rabbit sera by treatment group. Lines indicate the GMT +/- 95% confidence interval.

FIG. 26: EPA-specific IgG response by EPA dose.

### DETAILED DESCRIPTION OF THE INVENTION

### Definitions

To facilitate an understanding of the present invention, a number of terms and phrases are defined below. Alternate forms (tenses) of these terms and phrases are also encompassed herein. Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCR Publishers, Inc., 1995 (ISBN 1-56081-569-8).

“Comprise” (“comprising” or “comprises”) as used herein is open-ended and means “including, but not limited to.” “Having” is used herein as a synonym of comprising. It is understood that wherever embodiments are described herein with the language “comprising,” such embodiments encompass those described in terms of “consisting of” and/or “consisting essentially of”.

“Comprises therein” or “comprising therein” means that the referenced molecule, amino acid sequence, or nucleotide sequence has incorporated within it an O-linked glycosylation site molecule, amino acid sequence or nucleotide sequence, respectively. With respect to, for example, a “carrier protein comprising therein an O-linked glycosylation site,” the nucleotide sequence encoding that carrier protein has, between the 5’ and 3’ ends, a nucleotide sequence encoding a O-linked glycosylation site, likewise the carrier protein amino acid sequence has, between the N- and C- terminus, an O-linked glycosylation site amino acid sequence. “Protein carrier” or “Carrier protein” refers to a protein that comprises the consensus sequence into which the oligo- “poly″-saccharide is attached and external outer membrane of Gram-negative bacteria.

As used in the description and the appended claims, the singular forms “a,” “an,” and “the” include plural referents unless the context clearly dictates otherwise.

“About” or “approximately” mean roughly, around, or in the regions of. The terms “about” or “approximately” further mean within an acceptable contextual error range for the particular value as determined by one of ordinary skill in the art, which will depend in part on how the value is measured, i.e. the limitations of the measurement system or the degree of precision required for a particular purpose. When the terms “about” or “approximately” are used in conjunction with a numerical range, it modifies that range by extending the boundaries above and below the numerical values set forth.

The term “and/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless specifically stated, a process comprising a step of mixing two or more components does not require any specific order of mixing. Components can be mixed in any order. Where there are three components then two components can be combined with each other, and then the combination may be combined with the third component, etc. Similarly, while steps of a method may be numbered (such as (1), (2), (3), etc. or (i), (ii), (iii)), the numbering of the steps does not itself mean that the steps must be performed in that order (i.e., step 1 then step 2 then step 3, etc.). In certain embodiments, the word “then” is used to specify the order of a method’s steps.

“Essentially the same” herein means a high degree of similarity between at least two molecules (including structure or function) or numeric values such that one of skill in the art would consider the difference to be immaterial, negligible, and/or statistically insignificant. For example, a first polypeptide, conjugate, antibody, polynucleotide, vector, cell, composition, or molecule is “essentially the same” as a second polypeptide, conjugate, antibody, polynucleotide, vector, cell, composition, or molecule herein if the first has only immaterial differences in structure and function as compared to the second. “Essentially the same” herein encompasses “the same.”

An “effective amount” means an amount sufficient to cause the referenced effect or outcome. An “effective amount” can be determined empirically and in a routine manner using known techniques in relation to the stated purpose. In certain embodiments, a composition comprises an immunologically effective amount of an antigen, adjuvant, or both. In certain embodiments, an “effective amount” in the context of administering a therapy (e.g. an immunogenic composition or vaccine of the invention) to a subject refers to the amount of a therapy which has a prophylactic and/or therapeutic effect(s). In certain embodiments, an “effective amount” refers to the amount of a therapy which is sufficient to achieve one, two, three, four, or more of the following effects: (i) reduce or ameliorate the severity of a bacterial infection or symptom associated therewith; (ii) reduce the duration of a bacterial infection or symptom associated therewith; (iii) prevent the progression of a bacterial infection or symptom associated therewith; (iv) cause regression of a bacterial infection or symptom associated therewith; (v) prevent the development or onset of a bacterial infection, or symptom associated therewith; (vi) prevent the recurrence of a bacterial infection or symptom associated therewith; (vii) reduce organ failure associated with a bacterial infection; (viii) reduce hospitalization of a subject having a bacterial infection; (ix) reduce hospitalization length of a subject having a bacterial infection; (x) increase the survival of a subject with a bacterial infection; (xi) eliminate a bacterial infection in a subject; (xii) inhibit or reduce a bacterial replication in a subject; and/or (xiii) enhance or improve the prophylactic or therapeutic effect(s) of another therapy.

“Subject” refers to an animal, in particular a mammal such as a primate (e.g. human).

“Essentially free,” as in “essentially free from” or “essentially free of,” means comprising less than a detectable level of a referenced material or comprising only unavoidable levels of a referenced material (trace amounts).

The word “substantially” does not exclude “completely” e.g. a composition which is “substantially free” from Y may be completely free from Y. “Substantially pure” refers to material which is at least 50% pure (i.e., free from contaminants), at least 90% pure, at least 95% pure, at least 98% pure, or at least 99% pure.

As is conventional, the designation “NH₂” or “N-” refers to the N-terminus of an amino acid sequence and the designation “COOH” or “C-” refers to the C-terminus of an amino acid sequence.

“Internal”, “Interior” as used herein with respect to a protein, residue, or amino acid sequence means located between the N-terminus and the C-terminus.

“Fragment” is a nucleotide or polypeptide comprising “n” consecutive nucleic acids or amino acids, respectively, of the reference sequence and wherein “n” is any integer that is less than the total number of amino acids in the reference sequence. In certain embodiments, “n” is any integer between 1 and 100. In this way, a “fragment thereof” of a hypothetical 100 residue long reference sequence (SeqX) may consist of any 1 to 99 consecutive amino acids of SeqX. In certain embodiments, a fragment consists of 10, 20, 30, 40 or 50 contiguous amino acids of the full-length sequence. Fragments may be readily obtained by removing “n” consecutive amino acids from either or both of the N-terminus and C-terminus of the full-length reference polypeptide sequence. Fragments may be readily obtained by removing “n” consecutive nucleic acids from either or both of the 3′ and 5′ ends of the nucleotide sequence that encodes the full-length reference polypeptide sequence.

An “immunogenic fragment” as used herein consists of “n” consecutive amino acids of an antigen sequence and is capable of eliciting an antibody or immune response in a mammal. Fragments of a polypeptide, for example, can be produced using techniques known in the art, e.g. recombinantly, by proteolytic digestion, hydrolysis, energy (microwave, electrons, and other ions (MS), or by chemical synthesis. Internal or terminal fragments of a polypeptide can be generated by removing one or more nucleic acids from the 3’ or 5’ end (for a terminal fragment) or by removing one or more nucleic acids from both 3’ and 5’ ends (for an internal fragment) of a nucleotide sequence that encodes the polypeptide’s full-length amino acid sequence.

“Operably linked” or “operatively linked” means linked so as to be “operational”, for example, the configuration of polynucleotide sequences for recombinant protein expression. In certain embodiments, “operably linked” refers to the art-recognized positioning of, e.g., nucleic acid components such that the intended function (e.g., expression) is achieved. A person with ordinary skill in the art will recognize that under certain circumstances (e.g., a cleavage site or purification tag), two or more components “operably linked” together are not necessarily adjacent to each other in the nucleic acid or amino acid sequence (contiguously linked). A coding sequence that is “operably linked” to a “control sequence” (e.g., a promoter, enhancer, or IRES) is ligated in such a way that expression of the coding sequence is under the influence or control of the control sequence. A person with ordinary skill in the art will recognize that a variety of configurations are functional and encompassed.

“Recombinant” means artificial or synthetic. In certain embodiments, “recombinant” indicates the referenced amino acid, polypeptide, conjugate, antibody, nucleic acid, polynucleotide, vector, cell, composition, or molecule was made by an artificial combination of two or more molecules (e.g., heterologous nucleic acid or amino acid sequences). Such artificial combination includes, without limitation, chemical synthesis and genetic engineering techniques. In certain embodiments, a “recombinant polypeptide” refers to a polypeptide that has been made using recombinant nucleic acids (nucleic acids introduced into a host cell). In certain embodiments, a recombinant nucleic acid is not heterologous (e.g., wherein the recombinant nucleic acid is a second copy of a nucleic acid innately present within a host cell). A “transgene” herein means a polynucleotide introduced into a cell, therefore a transgene is recombinant.

The term “recombinant N-glycosylated protein” refers to any heterologous poly- or oligopeptide produced in a host cell that does not naturally comprise the nucleic acid encoding said protein. In the context of the present invention, this term refers to a protein produced recombinantly in any host cell, e.g. an eukaryotic or prokaryotic host cell, preferably a procaryotic host cell, e.g. Escherichia ssp., Campylobacter ssp., Salmonella ssp., Shigella ssp., Helicobacter ssp., Pseudomonas ssp., Bacillus ssp., more preferably Escherichia coli, Campylobacter jejuni, Salmonella typhimurium etc., wherein the nucleic acid encoding said protein has been introduced into said host cell and wherein the encoded protein is N-glycosylated by the OTase from Campylobacter spp., preferably C. jejuni, said transferase enzyme naturally occurring in or being introduced recombinantly into said host cell.

“Mutant” and “Modified” are given their well-understood and customary meanings and at least signify that the referenced molecule is altered (structure and/or function) as compared to control (e.g., wild type molecule or its naturally occurring counterpart) under comparable conditions or signify that the referenced numeric value is altered (increased or decreased) as compared to that of control under comparable conditions.

“Conservative” amino acid substitutions or mutations refer to the interchangeability of residues having similar side chains, and thus typically involves substitution of the amino acid in the polypeptide with amino acids within the same or similar defined class of amino acids. However, as used herein, in some embodiments, conservative mutations do not include substitutions from a hydrophilic to hydrophilic, hydrophobic to hydrophobic, hydroxyl-containing to hydroxyl-containing, or small to small residue, if the conservative mutation can instead be a substitution from an aliphatic to an aliphatic, non-polar to non-polar, polar to polar, acidic to acidic, basic to basic, aromatic to aromatic, or constrained to constrained residue. Further, as used herein, A, V, L, or l can be conservatively mutated to either another aliphatic residue or to another non-polar residue. The table below shows exemplary conservative substitutions.

TABLE 1

Conservative Substitutions

| | |
|---|---|
| Residue | Possible Conservative Mutations |
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| P | None |
| N, Q, S, T | Other polar |
| H, Y, W, F | Other aromatic (H, Y, W, F) |
| C | None |

As used herein, the term “deletion” is the removal of one or more amino acid residues from the protein sequence. Typically, no more than about from 1 to 6 residues (e.g. 1 to 4 residues) are deleted at any one site within the protein molecule.

As used herein, the term “insertion” is the addition of one or more non-native amino acid residues in the protein sequence. Typically, no more than about from 1 to 10 residues, (e.g. 1 to 7 residues, 1 to 6 residues, or 1 to 4 residues) are inserted at any one site within the protein molecule.

Hydrophilic amino acids herein include arginine (R), lysine (K), aspartic acid (D), glutamic acid (E), glutamine (Q), asparagine (N), histidine (H), serine (S), threonine (T), tyrosine (Y), cysteine (C), and tryptophan (W).

The term “any amino acids” is meant to encompass common and rare natural amino acids as well as synthetic amino acid derivatives and analogs that will still allow the optimized consensus sequence to be N-glycosylated by the OTase. Naturally occurring common and rare amino acids are preferred for X and Z. X and Z may be the same or different.

“Isolated” or “purified” herein means a polypeptide, conjugate, antibody, polynucleotide, vector, cell, composition, or molecule in a form not found in nature. This includes, for example, a polypeptide, conjugate, antibody, polynucleotide, vector, cell, composition, or molecule having been separated from host cell or organism (including crude extracts) or otherwise removed from its natural environment. In certain embodiments, an isolated or purified protein is a protein essentially free from all other polypeptides with which the protein is innately associated (or innately in contact with). For example, “isolated PgIL” or “purified PgIL” includes the recombinant PglL protein essentially free from other periplasmic polypeptides that the PglL protein would otherwise be associated with (in contact with) inside the host cell. For example, an “isolated O-glycosylated modified carrier protein” or “purified O-glycosylated modified carrier protein” may have been separated from un-O-glycosylated modified carrier protein (e.g., following in vitro conjugation steps). In certain embodiments, “isolated” or “purified” also means a protein is not bound to an antibody or antibody fragment. In certain embodiments, an isolated or purified protein does not include a collection of the protein’s components (sub-parts). For example, wherein the protein is a complex of protein components, an “isolated/purified complex” may not include a collection of the complex’s components (unbound to each other) obtained after, for example, application of sodium dodecyl sulfate (SDS) or 2-Mercaptoethanol (both of which break down the bonds between protein components in a complex).

A “Pharmaceutical-grade” or “pharmaceutically acceptable” polypeptide, conjugate, antibody, polynucleotide, vector, cell, composition, or molecule is isolated, purified, or otherwise formulated to be essentially free from impurities (e.g., essentially free from components (e.g., naturally occurring components) which are unacceptably toxic to a subject to which the polypeptide, conjugate, antibody, polynucleotide, vector, cell, composition, or molecule may be administered). A pharmaceutical-grade polypeptide, conjugate, antibody, polynucleotide, vector, cell, composition, or molecule is not a crude polypeptide, conjugate, antibody, polynucleotide, vector, cell, composition, or molecule.

“Homologue(s)” as used herein means two or more molecules that, despite originating from a different genus or species of organism and/or having divergent structure, have essentially the same function. To denote similar functionality herein, “PgIL” or “PilE” may be used to refer to oligosaccharyltransferases or pilin, respectively, even if alternate designations are used in the art.

“Endogenous” as used herein means the referenced two or more polypeptides, conjugates, antibodies, polynucleotides, vectors, cells, compositions, or molecules originate from the same species of organism, or, in the case of a synthetic or recombinant polypeptide for example, consists essentially of the structure and function as those that originate from the same species of organism. With respect to PgIL, for example, “endogenous” refers to the relationship of the subject PglL to the subject pilin (or O-linked glycosylation site therefrom) and means that they both originate from the same species of organism or consist essentially of the structure and function as those that originate from the same species of organism. As an example, a Neisseria meningitidis PglL is “endogenous” to N. meningitidis PilE (and in this way, a PglL may be said to be “endogenous to” the referenced pilin). As a further example, a Neisseria meningitidis PglL is “endogenous to” N. meningitidis cells (especially control or wild type N. meningitidis cells).

“Heterologous” as used herein means the referenced two or more things are not associated with each other in nature. In certain embodiments, a protein is “heterologous” to a cell if a comparable naturally occurring cell (e.g., wild type cell under comparable conditions) would not produce that protein. In certain embodiments, a periplasmic signal sequence is “heterologous” to a protein (or to the protein’s amino acid sequence) because the comparable naturally occurring protein (e.g., wild type protein) would not be operatively linked to that signal sequence.

“Nucleic acid,” “nucleotide,” “polynucleotide” is used to refer to ribonucleic acid (RNA), deoxyribonucleic acid (DNA), a polyribonucleotide molecule, or a polydeoxyribonucleotide molecule whether or not modified, unmodified, or synthetic. Thus, polynucleotides as defined herein may include single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. Thus, DNAs or RNAs with backbones modified for stability or for other reasons are “polynucleotides” as that term is intended herein. DNAs or RNAs may be synthetic (including, without limitation, the nucleic acid subunits that together form the polynucleotide). Moreover, DNAs or RNAs comprising unusual bases, such as inosine, or modified bases, such as tritiated bases, are included within the term “polynucleotides” as defined herein.

In general, the term “polynucleotide” embraces all chemically, enzymatically and/or metabolically modified forms of unmodified polynucleotides. Polynucleotides can be made by a variety of methods, including in vitro recombinant DNA-mediated techniques and by expression of DNAs in cells and organisms. Polynucleotides include genomic and plasmid nucleic acids. DNA includes, without limitation, genomic (nuclear) DNA having introns, e.g., as well as recombinant DNA such as cDNA (e.g., introns removed). RNA includes, without limitation, mRNA and tRNA. It is envisioned that codon optimization is utilized for any recombinant expression of a polynucleotide molecule of the present invention.

“Vector” refers to a vehicle by which nucleic acid molecules are contained and transferred from one environment to another or that facilitates the manipulation of a nucleic acid molecule. A vector may be, for example, a cloning vector, an expression vector, or a plasmid. Vectors include, for example, a BAC or a YAC vector. The term “expression vector” includes, without limitation, any vector, (e.g., a plasmid, cosmid or phage chromosome) containing a coding sequence suitable for expression by a cell (e.g., wherein the coding sequence is operatively linked to a transcriptional control element such as a promoter). A vector may comprise two or more nucleic acid molecules, in certain embodiments each of those two or more nucleic acid molecules comprises a nucleotide sequence that encodes a protein.

“Polypeptide” and “protein” are used interchangeably herein to refer to polymers of amino acids of any length. “Peptide” may be used to refer to a polymer of amino acids consisting of 1 to 50 amino acids. The polymer can be linear or branched, it can comprise modified amino acids, and it can be interrupted by non-amino acids. The terms also encompass an amino acid polymer that has been modified naturally or by intervention; for example, disulfide bond formation, glycosylation (except the O-glycosylation of modified carrier proteins), lipidation, acetylation, phosphorylation, amidation, derivatization by known protecting/blocking groups, proteolytic cleavage, modification by non-naturally occurring amino acids, or any other manipulation or modification, such as conjugation with a labeling component. Also included within the definition are, for example, polypeptides containing one or more analogs of an amino acid (including, for example, unnatural amino acids, etc.), as well as other modifications known in the art.

A “Glycan” is a large carbohydrate molecule containing smaller sugar molecules and in certain embodiments herein refers to the oligosaccharide chain of a “glycoprotein” (a protein comprising glycan(s) covalently attached to amino acid side chains). “O-glycan” or “O-linked-glycan” is used herein to reference a glycan that is covalently attached to a serine or threonine residue of another molecule (i.e., the glycan is engaged in o-linked glycosylation). Glycans may be immunogenic. A glycan is any sugar that can be transferred (e.g, covalently attached) to a carrier protein. A glycan comprises monosaccharides, oligosaccharides and polysaccharides. An oligosaccharide is a glycan having 2 to 10 monosaccharides. A polysaccharide is a glycan having greater than 10 monosaccharides. Polysaccharides can be selected from the group consisting of O-antigens, capsules, and exopolysaccharides.

Glycans for use with the present invention are PglL Otase substrates. [3], [29], [30], [31], [32], and [33]. In certain embodiments, the glycan is operably linked to a lipid-carrier. In certain embodiments, the glycan can be, but is not limited to, hexoses, N-acetyl derivatives of hexoses, oligosaccharides, and polysaccharides. In certain embodiments, the monosaccharide at the reducing end of the glycan is a hexose or an N-acetyl derivative of a hexose. In a certain embodiment, the glycan comprises a hexose monosaccharide at its reducing end such as glucose, galactose, rhamnose, arabinotol, fucose or mannose. In certain embodiments, the hexose monosaccharide at the reducing end is glucose or galactose. In certain embodiments, the reducing end of the glycan is an N-acetyl derivative of hexose. In general, N-acetyl derivatives of hexose (or “hexose monosaccharide derivatives”) comprise an acetamido group at position 2. In certain embodiments, N-acetyl derivatives of hexose is selected from N-acetylglucosamine (GlcNAc), N-acetylhexosamine (HexNAc), deoxy HexNAc, and 2,4-diacetamido-2,4,6-trideoxyhexose (DATDH), N-acetylfucoseamine (FucNAc), and N-acetylquinovosamine (QuiNAc). In certain embodiments, the N-acetyl derivative of hexose is selected from N-acetylglucosamine (GlcNAc), N-acetylgalactosamine (GalNAc), N-acetylfucoseamine (FucNAc), 2,4-diacetarnido-2,4,6-trideoxyhexose (DATDH), glyceramido-acetamido trideoxyhexose (GATDH), and N-acetylhexosamine (HexNAc). In certain embodiments, the glycan has a reducing end of N,N-diacetylbacillosamine (diNAcBac) or Pseudaminic acid (Pse). In certain embodiments, the glycan is one that has a reducing end of Glucose, Galactose, arabinotol, fucose, mannose, Galactofuranose, Rhamnose, GlcNAc, GalNAc, FucNAc, DATDH, GATDH, HexNAc, deoxy HexNAc, QuiNAc, diNAcBac, or Pse. In certain embodiments, the glycan is one that has a reducing end of Glucose, Galactose, GlcNAc, GalNAc, FucNAc, DATDH, GATDH, HexNAc, deoxy HexNAc, or diNAcBac. In certain embodiments, the glycan is one that has a reducing end of Glucose, Galactose, Galactofuranose, Rhamnose, GlcNAc, GalNAc, FucNAc, DATDH, GATDH, or diNAcBac. In certain embodiments, the glycan is one that has a reducing end of Glucose, Galactose, GlcNAc, GalNAc, FucNAc, DATDH, GATDH, or diNAcBac. In certain embodiments, the glycan is one that has a reducing end selected from the group consisting of DATDH, GlcNAc, GalNAc, FucNAc, Galactose, and Glucose. In certain embodiments, the glycan is one that has a reducing end GlcNAc, GalNAc, FucNAc, or Glucose. In certain embodiments, the glycan is one that has a S-2 to S-1 reducing end of Galactose-β1,4-Glucose; Glucuronic acid-β1,4-glucose; N-acetyl-fucosamine-α1,3-N-acetyl-galactosamine; Galactose-β1,4-glucose; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine.

In certain embodiments, the glycan is endogenous to a Neisseria, Shigella, Salmonella, Streptococcus, Escherichia, Pseudomonas, Yersinia, Campylobacter, or Heliobacter cell. In certain embodiments, the glycan is endogenous to a Shigella, Salmonella, Escherichia, or Pseudomonas cell. In certain embodiments, the glycan is endogenous to a Shigella flexneri, Salmonella paratyphi, Salmonella enterica, or E. coli cell. In certain embodiments, the glycan is from C. jejuni, N. meningitidis, P. aeruginosa, S. enterica LT2, or E. coli. See [4], [29], [3], [34].

In certain embodiments, the glycan is an immunogenic glycan (an antigen). In certain embodiments, the glycan is an O-antigen. In certain embodiments, the glycan is an immunogenic O-antigen endogenous to a Neisseria, Shigella, Salmonella, Streptococcus, Escherichia, Pseudomonas, Yersinia, Campylobacter, or Heliobacter cell. In further embodiments, the PglL Glycan Substrate is a Shigella sonnei glycan antigen e.g. S. sonnei O-antigen, a Shigella flexneri glycan antigen e.g. Shigella flexneri 2a CPS, a Shigella dysenteriae glycan antigen, a Streptococcus pneumoniae glycan antigen e.g. Streptococcus pneumoniae sp. 12F CPS, S. pneumoniae sp. 8 CPS, S. pneumoniae sp. 14 CPS, S. pneumoniae sp. 23A CPS, S. pneumoniae sp. 33F CPS, or S. pneumoniae sp. 22A CPS. In certain embodiments, the glycan is a Streptococcus pneumoniae glycan having a reducing end of Glucose, Galactose, arabinotol, fucose, mannose, Galactofuranose, Rhamnose, GlcNAc, GalNAc, FucNAc, DATDH, GATDH, HexNAc, deoxy HexNAc, QuiNAc, diNAcBac, or Pse. In certain embodiments, the glycan is a Streptococcus pneumoniae glycan is one that has a S-2 to S-1 reducing end of Galactose-β1,4-Glucose; Glucuronic acid-β1,4-glucose; N-acetyl-fucosamine-α1,3-N-acetyl-galactosamine; Galactose-β1,4-glucose; Rhamnose-β1,4-glucose; Galactofuranose-β1,3-glucose; N-acetyl-altruronic acid-a1,3-4-amino-N-acetyl-fucosamine; or Rhamnose-β1,4-N-acetylgalactosamine. The CP gene clusters of all 90 S. pneumoniae serotypes have been sequenced by Sanger Institute (available at WorldWideWeb(www).sanger.ac.uk/Projects/S_pneumoniae/CPS/). Sequences are provided in NCBI as Genbank CR931632-CR931722. The capsular biosynthetic genes of S. pneumoniae are further described in Serotype 23A from Streptococcus pneumoniae strain 1196/45 (serotype 23a) as NCBI GenBank accession number: CR931683.1. Serotype 23B from Streptococcus pneumoniae strain 1039/41 as NCBI GenBank accession number: CR931684.1. Serotype 23F from Streptococcus pneumoniae strain Dr. Melchior as NCBI GenBank accession number: CR931685.1.

In certain embodiments, the glycan is an S. sonnei O-antigen. In certain embodiments, the S. sonnei O-antigen consists of a wbgT protein, a wbgU protein, a wzx protein, a wzy protein, a wbgV protein, a wbgW protein, a wbgX protein, a wbgY protein, and a wbgZ protein. In certain embodiments, the S. sonnei O-antigen consists of a wbgT protein having at least 90% identity to SEQ ID NO: 3, a wbgU protein having at least 90% identity to SEQ ID NO: 4, a wzx protein having at least 90% identity to SEQ ID NO: 5, a wzy protein having at least 90% identity to SEQ ID NO: 6, a wbgV protein having at least 90% identity to SEQ ID NO: 7, a wbgW protein having at least 90% identity to SEQ ID NO: 8, a wbgX protein having at least 90% identity to SEQ ID NO: 9, a wbgY protein having at least 90% identity to SEQ ID NO: 10, and a wbgZ protein having at least 90% identity to SEQ ID NO: 11).

“Homogeneity” means the variability of glycan length and possibly the number of glycosylation sites. Methods listed above can be used for this purpose. SE-HPLC allows the measurement of the hydrodynamic radius. Higher numbers of glycosylation sites in the carrier lead to higher variation in hydrodynamic radius compared to a carrier with less glycosylation sites. However, when single glycan chains are analyzed, they may be more homogenous due to the more controlled length. Glycan length is measured by hydrazinolysis, SDS PAGE, and CGE. In addition, homogeneity can also mean that certain glycosylation site usage patterns change to a broader/narrower range. These factors can be measured by Glycopeptide LC-MS/MS.

“Bioconjugate homogeneity” means the homogeneity of the attached sugar residues and can be assessed using methods that measure glycan length and hydrodynamic radius.

“Reducing end” of an oligosaccharide or polysaccharide is the monosaccharide with a free anomeric carbon that is not involved in a glycosidic bond and is thus capable of converting to the open-chain form. The first sugar (“S-1”) herein is that comprising the reducing end and the second sugar (“S-2”) is that which is adjacent to S-1. The S-2 sugar may be attached to the S-1 sugar by, for example, an α-(1→3), β-(1→3), β-(1→4), or α-(1→6) linkage.

“Glycosyltransferases” (GTFs, Gtfs) are enzymes that establish glycosidic linkages. Glycosyltransferases are enzymes that catalyze the formation of the glycosidic linkage to form a glycoside. For example, they catalyze the transfer of saccharide moieties from an activated nucleotide sugar (also known as the “glycosyl donor”) to a nucleophilic glycosyl acceptor molecule, the nucleophile of which can be oxygen- carbon-, nitrogen-, or sulfur-based.

“O-Antigens” (also known as O-specific polysaccharides or O-side chains) are a component of the surface lipopolysaccharide (LPS) of Gram-negative bacteria. Examples include O-antigens from Pseudomonas aeruginosa and Klebsiella pneumoniae.

“O-glycosylated modified carrier protein” means the modified carrier protein is glycosylated and, in particular, is engaged in O-linked glycosylation (e.g., a modified carrier protein that is O-linked to a PglL Glycan Substrate).

An O-glycosylated modified carrier protein may be directly or indirectly attached to two or more distinct immunogenic glycans and, in this way, useful for inducing an immune or antibody response to the two or more immunogenic glycans (i.e., multivalent).

The use of multiple O-linked glycosylation site s within one carrier protein is envisioned (see Examples), optionally, multiple O-linked glycosylation site s being adjacent to each other. Two or more O-linked glycosylation site s may be separated by a “Amino Acid Linker” consisting of one or more amino acids, which can be, for example, one or more glycine ( [26]), one or more serine, and/or combinations thereof (See [27]). An “amino acid linker” herein is a type of “linker”.

O-glycosylation efficiency of O-linked glycosylation site s located at the N- or C-terminus of a carrier protein may be increased by flanking the O-linked glycosylation site (i.e., placing toward the N-terminus and/or toward the C-terminus of the O-linked glycosylation site) with one or more “Flanking Peptide” (a peptide comprising hydrophilic amino acids such as, for example, DPRNVGGDLD (residues 599-608 of SEQ ID NO: 12) or QPGKPPR (residues 628-634 of SEQ ID NO: 12)). [28]. Such Flanking Peptide may be adjacent to the O-linked glycosylation site (i.e., with no amino acids between the O-linked glycosylation site and the Flanking Peptide) or may have 1, 2, 3, 4, 5, 6, 7, 8, 9, or 10 amino acids between it and the O-linked glycosylation site. An insertion of two or more Flanking Peptides can be used. Flanking Peptides can be used to increase the O-glycosylation efficiency of shorter O-linked glycosylation site s, such as those having the sequence SEQ ID NO: 13, 14, 15, or 16 (all 12 amino acids long).

“Lipopolysaccharide” (LPS), also known as lipoglycans, are large molecules consisting of a lipid and a polysaccharide joined by a covalent bond; they are found in the outer membrane of Gram-negative bacteria, act as endotoxins and elicit strong immune responses in animals.

N-glycans or N-linked oligosaccharides refer to mono-, oligo- or polysaccharides of variable compositions that are linked to an ε-amide nitrogen of an asparagine residue in a protein via an N-glycosidic linkage.

N-linked protein glycosylation refers to a process or pathway to link covalently “glycans” (mono-, oligo- or polysaccharides) to a nitrogen of asparagine (N) side-chain on a target protein.

O-antigens refers to a repetitive glycan polymer contained within an LPS, also called O-polysaccharide. The O antigen is attached to the core oligosaccharide and comprises the outermost domain of the LPS molecule.

Oligosaccharides or Polysaccharides refers to homo or heteropolymer formed by covalently bound carbohydrates (monosaccharides) consisting of repeating units (monosaccharides, disaccharides, trisaccharides, etc.) linked together by glycosidic bonds.

OTase or OST refers to oligosaccharyl transferase which catalyzes a mechanistically unique and selective transfer of an oligo- or polysaccharide (glycosylation) to the asparagine (N) residue at the consensus sequence of nascent or folded proteins.

“Capsular polysaccharide” (CP) is a polysaccharide found on the bacterial cell wall. Examples include capsular polysaccharide from Streptococcus pneumoniae, Haemophilus influenzae, Neisseria meningitidis and Staphylcoccus aureus.

“wzy” is a polysaccharide polymerase gene encoding an enzyme which catalyzes polysaccharide polymerization. The encoded enzyme transfers oligosaccharide units to the non-reducing end forming a glycosidic bond.

“waaL” is an O antigen ligase gene encoding a membrane bound enzyme. The encoded enzyme transfers undecaprenyl-diphosphate (UPP)-bound O antigen to the lipid A core oligosaccharide, forming lipopolysaccharide.

As used herein, the term “bioconjugate” refers to conjugate between a protein (e.g. a carrier protein) and an antigen (e.g. a saccharide antigen, such as a bacterial polysaccharide antigen) prepared in a host cell background, wherein host cell machinery links the antigen to the protein (e.g. N-linked glycosylation).

As used herein, the term “modified protein” means a protein that is altered (in one or more way) as compared to wild type (e.g. a “modified EPA protein” excludes a wild type EPA protein”).

As used herein, the term “subject” refers to an animal, in particular a mammal such as a primate (e.g. human).

“Antigen” or “immunogen” herein refer to a substance, typically a protein or glycan, which is capable of inducing an immune response in a subject. In certain embodiments, an antigen is a protein (e.g., a glycoprotein) that is “immunologically active,” meaning that once administered to a subject (either directly or by administering to the subject a nucleotide sequence or vector that encodes the protein) it is able to evoke an immune response of the humoral and/or cellular type directed against that protein. “O-antigens” consist of repeats of an oligosaccharide unit (O-unit), which generally has between two and six sugar residues. O-antigens are components of the outer-membrane of gram-negative bacteria. In certain embodiments, the glycan is an O-antigen.

“Adjuvants” are substances that enhance the induction, magnitude, and/or longevity of an antigen’s immunological effect.

Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see United Kingdom Patent GB2220211), MF59 (Novartis), AS03 (GlaxoSmithKline), AS04 (GlaxoSmithKline), polysorbate 80 (Tween 80; ICL Americas, Inc.), imidazopyridine compounds [35] and saponins, such as QS21 [36]. Suitable adjuvants include an aluminum salt such as aluminum hydroxide gel (alum) or aluminium phosphate, but may also be a salt of calcium, magnesium, iron or zinc, or may be an insoluble suspension of acylated tyrosine, or acylated sugars, cationically or anionically derivatized polysaccharides, or polyphosphazenes.

“Conjugation” references the coupling of carrier protein to saccharide (e.g., by covalent bond).

“Conjugate” herein means two or more molecules (e.g., proteins) which are attached to each other. The two or molecules are optionally recombinant molecules and/or are heterologous to each other. In certain embodiments, the conjugate comprises two or more molecules, the first being a carrier protein, for example a modified carrier protein, and the remaining one or more molecules being glycans covalently attached to a serine or threonine residue of the carrier protein. In certain embodiments, a conjugate comprises a glycosylated carrier protein, such as an O-glycosylated carrier protein, including an O-glycosylated modified carrier protein. A conjugate may be the result of chemical conjugation or in vitro conjugation (bioconjugation).

“Antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, multispecific antibodies such as bispecific antibodies generated from at least two intact antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment” refers to a portion of an intact antibody that binds to an antigen. An antigen-binding fragment can contain the antigenic determining variable regions of an intact antibody. Examples of antibody fragments include, but are not limited to Fab, Fab’, F(ab’)2, and Fv fragments, linear antibodies, and single chain antibodies.

“Antibody response” means production of an anti-antigen antibody. “Inducing an antibody response” or “raising an antibody response” means stimulating in vivo the production of an anti-antigen antibody, e.g., an anti-O-antigen antibody or an anti-glycan-antibody.

“Percentage of sequence identity,” “percent identity,” and “percent identical” are used herein to refer to comparisons between polynucleotide sequences or polypeptide sequences, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage is calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Determination of optimal alignment and percent sequence identity is performed using the BLAST and BLAST 2.0 algorithms (see, e.g., Altschul, et al., 1990, J. Mol. Biol. 215: 403-410 and Altschul, et al., 1977, Nucleic Acids Res. 3389-3402). Software for performing BLAST analyses is publicly available through the National Center for Biotechnology Information website.

Briefly, the BLAST analyses involve first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul, et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=-4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915).

Numerous other algorithms are available that function similarly to BLAST in providing percent identity for two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel, et al., eds., Current Protocols, a joint venture between Greene Publishing Associates, Inc. and John Wiley & Sons, Inc., (1995 Supplement) (Ausubel)). Additionally, determination of sequence alignment and percent sequence identity can employ the BESTFIT or GAP programs in the GCG Wisconsin Software package (Accelrys, Madison WI), using default parameters provided. The ClustalW program is also suitable for determining identity.

As a non-limiting example, whether any particular polynucleotide or polypeptide has a certain percentage sequence identity (e.g., is at least 80% identical, at least 85% identical, at least 90% identical, and in some embodiments, at least 90%, 91%, 92%, 93%, 94%, 95%, 96%, 97%, 98%, or 99% identical) to a reference sequence can, be determined using known methods such as the Bestfit program (Wisconsin Sequence Analysis Package, Version 8 for Unix, Genetics Computer Group, University Research Park, 575 Science Drive, Madison, Wl 53711). Best fit uses the local homology algorithm of Smith and Waterman (Advances in Applied Mathematics 2: 482 489 (1981)) to find the best segment of homology between two sequences. When using Bestfit or any other sequence alignment program to determine whether a particular sequence is, for instance, 95% identical to a reference sequence according to the present invention, the parameters are set such that the percentage of identity is calculated over the full length of the reference nucleotide sequence and that gaps in homology of up to 5% of the total number of nucleotides in the reference sequence are allowed.

In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. Identity can exist over a region of the sequences that is at least about 10, about 20, about 40-60 residues in length or any integral value there between, and can be over a longer region than 60-80 residues, for example, at least about 90-100 residues, and in some embodiments, the sequences are substantially identical “over the full length of” the sequences being compared, such as the coding region of a nucleotide sequence for example.

“Numbered with respect to”, “as compared to”, “numbered according to” is used herein to reference a location in an amino acid sequence while not being limited to that referenced amino acid sequence. It would therefore be understood, for example, that residue “I28 numbered with respect to SEQ ID NO: 17” may encompass I29 of SEQ ID NO: 18 as well as l28 of SEQ ID NO: 19 (demonstrated below).

SEQ_ID_NO_17 SAVTEYYLNHGEWPGNNTSAGVA
TS-SEIK------ 29

SEQ_ID_NO_18 SAVTGYYLNHGTWPKDNTSAGVA
SSPTDIK------ 30

SEQ_ID_NO_19 GAVTEYEADKGVFPTSNASAGVA
AA-ADINGK---- 31

“Host cell” as used herein refers to a cell into which a molecule (usually a heterologous or non-native nucleic acid molecule) is, has been, or will be introduced. A host cell herein does not encompass a whole human organism.

Oligosaccharyltransferases (OSTs or OTases) are membrane-embedded enzymes that transfer oligosaccharides from a lipid carrier to a nascent protein (a type of glycosyltransferase). O-linked glycosylation consists of the covalent attachment of a sugar molecule (a glycan) to a side-chain hydroxyl group of an amino acid residue (e.g. serine, or threonine) in the protein target (e.g., pilin).

“Carrier protein” as used herein means a protein suitable for use as a carrier protein in the production of bioconjugate vaccines (e.g., [32]). “Carrier protein” as used herein is distinct from a “lipid carrier” (or “lipid-linked-carrier”), the latter of which include, without limitation, undecaprenyl-pyrophosphate (UndPP). “Carrier protein” may be covalently attached to an antigen (e.g. saccharide antigen, such as a bacterial polysaccharide antigen) to create a conjugate (e.g. bioconjugate). A carrier protein activates T-cell mediated immunity in relation to the antigen to which it is conjugated.

Any carrier protein suitable for use in the production of conjugate vaccines (e.g., bioconjugates for use in vaccines) can be used herein, e.g., nucleic acids encoding the carrier protein can be introduced into a host provided herein for the production of a bioconjugate comprising a carrier protein linked to Pseudomonas antigen. Exemplary carrier proteins include, without limitation, detoxified Exotoxin A of P. aeruginosa (EPA; see, e.g., Ihssen, et al., (2010) Microbial cell factories 9, 61), CRM197, maltose binding protein (MBP), Diphtheria toxoid, Tetanus toxoid, detoxified hemolysin A of S. aureus, clumping factor A, clumping factor B, E. coli FimH, E. coli FimHC, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin and detoxified variants thereof, C. jejuni AcrA, Pseudomonas PcrV protein, and C. jejuni natural glcyoproteins.

A “modified carrier protein” as used herein means a carrier protein that is altered (in one or more way) as compared to wild type (i.e., a “modified carrier protein” excludes a wild type pilin protein). A modified carrier protein includes, without limitation, a carrier protein incorporating one or more O-linked glycosylation site(s), purification tag, deletion (e.g., of at least a part of the transmembrane domain), insertion, and/or mutation (e.g., AcrA mutation(s) ([22]). In certain embodiments, the modified carrier protein is altered as compared to a control carrier protein (e.g., wild type) such that the modified carrier protein may be an “acceptor” of the PglL Glycan Substrate (i.e., accept the PglL Glycan Substrate directly from PglL without pilin intermediate). In certain embodiments, one such modified carrier protein is altered by comprising one or more O-linked glycosylation site s. In certain embodiments, one such modified carrier protein comprises one or more O-linked glycosylation site s at its N-terminus, C-terminus, and/or interior residues. For clarity, “a modified carrier protein comprising a carrier protein having one or more O-linked glycosylation site s at its N-terminus and/or C-terminus” means “a modified carrier protein comprising a carrier protein operably linked to one or more O-linked glycosylation site s at its N-terminus and/or C-terminus.”

In certain embodiments, the modified carrier protein is covalently coupled to a glycan, either directly (e.g., via an O-linked glycosidic bond) or indirectly (e.g., via a linker), wherein the coupling is at one or more of the O-linked glycosylation site s. In further embodiments, the glycan is a PglL Glycan Substrate.

In certain embodiments, the modified carrier protein is coupled to a Shigella glycan (e.g. a Shigella sonnei glycan (such as S. sonnei O-antigen), or e.g. a Shigella flexneri glycan (such as Shigella flexneri 2a CPS), or a Shigella dysenteriae glycan).

In certain embodiments, the modified carrier protein is coupled to a Streptococcus glycan (e.g. Streptococcus pneumoniae (such as Streptococcus pneumoniae sp. 12F CPS, S. pneumoniae sp. 8 CPS, S. pneumoniae sp. 14 CPS, S. pneumoniae sp. 23A CPS, S. pneumoniae sp. 33F CPS, or S. pneumoniae sp. 22A CPS)).

In certain embodiments, the PglL OTase is a Neisseria meningitidis PglL, Neisseria gonorrhoeae PglL, Neisseria lactamica 020-06 PglL, Neisseria lactamica ATCC 23970 PglL, Neisseria gonorrhoeae F62 PglL, Neisseria cinerea ATCC 14685 PglL, Neisseria mucosa PglL, Neisseria flavescens NRL30031/H210 S. pneumoniae PgIL, Neisseria mucosa ATCC 25996 PglL, Neisseria sp. oral taxon 014 strain F0314 PglL, Neisseria arctica PgIL, Neisseria shayeganii 871 PglL, Neisseria shayeganii 871 PgIL, Neisseria sp. 83E34 PgIL, Neisseria wadsworthii PglL, Neisseria elongata subsp. glycolytica ATCC 29315 PglL, Neisseria bacilliformis ATCC BAA-1200 PglL, Neisseria sp. oral taxon 020 str. F0370 PgIL, Neisseria sp. 74A18 PglL, Neisseria weaver ATCC 51223 PglL, or Neisseria macacae ATCC 33926 PglL OTase.

In certain embodiments, the PglL Glycan Substrate is an O-antigen. In certain embodiments, the PglL Glycan Substrate is S. sonnei O-antigen.

Exemplary carrier proteins include, without limitation, detoxified Exotoxin A of P. aeruginosa (“EPA”; see, e.g., [6]), CRM197, maltose binding protein (MBP), Diphtheria toxoid (DT), Tetanus toxoid (TT), Tetanus Toxin C fragment (TTc), detoxified hemolysin A of S. aureus, clumping factor A, clumping factor B, E. coli FirmH, E. coli FirmHC, E. coli heat labile enterotoxin, detoxified variants of E. coli heat labile enterotoxin, Cholera toxin B subunit (CTB), cholera toxin, detoxified variants of cholera toxin, E. coli Sat protein, the passenger domain of E. coli Sat protein, Streptococcus pneumoniae Pneumolysin and detoxified variants thereof, C. jejuni Acriflavine resistance protein A (CjAcrA), E. coli Acriflavine resistance protein A (EcAcrA), Pseudomonas aeruginosa PcrV protein (PcrV), C. jejuni natural glycoproteins, S. pneumoniae NOX, S. pneumoniae PspA, S. pneumoniae PcpA, S. pneumoniae PhtD, S. pneumoniae PhtE, S. pneumoniae ply (e.g. detoxified ply), S. pneumoniae LytB, Haemophilus influenzae protein D (PD). [23], [24], [25]. In certain embodiments, the carrier protein is selected from the group consisting of CTB, TT, TTc, DT, CRM197, EPA, EcAcrA, CjAcrA, and PcrV. In certain embodiments, the carrier protein is selected from the group consisting of EPA, EcAcrA, CjAcrA, and PcrV. In certain embodiments, the carrier protein is EPA. In certain embodiments, the carrier protein is EcAcrA.

A “purification tag” as used herein refers to a ligand that aids protein purification with, for example, size exclusion chromatography, ion exchange chromatography, and/or affinity chromatography. Purification tags and their use are well known to the art and may be, for example, poly-histidine (HIS), glutathione S-transferase (GST), c-Myc (Myc), hemagglutinin (HA), FLAG, or maltose binding protein (MBP). In certain embodiments, a purification tag is an epitope tag (which include, e.g., a histidine, FLAG, HA, Myc, V5, Green Fluorescent Protein (GFP), β-galactosidase (b-GAL), luciferase, Maltose Binding Protein (MBP), or Red Fluorescence Protein (RFP) tag). In certain embodiments, polypeptides are operably linked to one or more purification tags (including combinations of purification tags). A step of purifying, collecting, obtaining, or isolating a protein may therefore include size exclusion chromatography, ion exchange chromatography, or affinity chromatography. In certain embodiments, a step of purifying a modified carrier protein (or a conjugate comprising it), utilizes affinity chromatography and, for example, a σ28 affinity column or an affinity column comprising an antibody that binds the modified carrier protein or the conjugate comprising it (optionally by binding to the glycn). In a certain embodiment, a step of purifying a fusion protein comprising at least a modified carrier protein operably linked to a purification tag utilizes affinity chromatography and, for example, an affinity column that binds the purification tag.

“Cell substrates” refers to the cells that are used to produce the desired biotechnological/biological products.

“Yield” is measured as carbohydrate amount derived from a liter of bacterial production culture grown in a bioreactor under controlled and optimized conditions. After purification of bioconjugate, the carbohydrate yields can be directly measured by either the anthrone assay or ELISA using carbohydrate specific antisera. Indirect measurements are possible by using the protein amount (measured by BCA, Lowry, or bardford assays) and the glycan length and structure to calculate a theoretical carbohydrate amount per gram of protein. In addition, yield can also be measured by drying the glycoprotein preparation from a volatile buffer and using a balance to measure the weight.

An “immunogenic composition”, “vaccine composition,” or “pharmaceutical composition” is a preparation formulated to permit the biological activity of the active ingredient to be effective, and which contains no additional components which are unacceptably toxic to a subject to which the composition would be administered. Immunogenic, vaccine, or pharmaceutical compositions comprise pharmaceutical-grade active ingredients (e.g., pharmaceutical-grade antigen), therefore, the immunogenic, vaccine, or pharmaceutical compositions of the present invention are distinguished from any, e.g., naturally occurring composition. See [34]. In certain embodiments, the immunogenic, vaccine, or pharmaceutical composition is sterile. In certain embodiments, the composition is an immunogenic composition comprising an “immunogenic conjugate” (e.g., a modified carrier protein covalently linked to an immunogenic glycan). In certain embodiments, the immunogenic glycan is an O-antigen. Immunogenic compositions comprise an immunologically effective amount of the immunogenic glycan or immunogenic conjugate.

An “immunologicaly effective amount” may be administered to an individual as a single dose or as part of a series. In certain embodiments, the immunogenic composition further comprises a pharmaceutically acceptable adjuvant, excipient, carrier, or diluent. Adjuvants, excipients, carriers, and diluents do not themselves induce an antibody or immune response, but rather they provide the technical effect of eliciting or enhancing an antibody or immune response to an antigen (e.g., an immunogenic glycan).

“Conjugate vaccine” refers to a vaccine created by covalently attaching a polysaccharide antigen to a carrier protein. Conjugate vaccine elicits antibacterial immune responses and immunological memory. In infants and elderly people, a protective immune response against polysaccharide antigens can be induced if these antigens are conjugated with proteins that induce a T-cell dependent response.

Consensus sequence refers to a sequence of amino acids, -D/E - X - N - Z - S/T-wherein X and Z may be any natural amino acid except Proline, within which the site of carbohydrate attachment to N-linked glycoproteins is found.

Capsular polysaccharide refers to a thick, mucous-like, layer of polysaccharide. Capsular polysaccharides are water soluble; commonly acidic that consist of regularly repeating units of one to several monosaccharides/monomers.

Glycoconjugate vaccine refers to a vaccine consisting of a protein carrier linked to an antigenic oligosaccharide.

Glycosyltransferase refers to enzymes that act as a catalyst for the transfer of a monosaccharide unit from an activated nucleotide sugar to a glycosyl acceptor molecule.

Gram-positive strain refers to a bacterial strain that stains purple with Gram staining (a valuable diagnostic tool). Gram-positive bacteria have a thick mesh-like cell wall made of peptidoglycan (50-90% of cell wall).

Gram-negative strain refers to a bacterial strain which has a thinner layer (10% of cell wall) which stains pink. Gram-negative bacteria also have an additional outer membrane that contains lipids and is separated from the cell wall by the periplasmic space.

Passive immunization is the transfer of active humoral immunity in the form of already made antibodies, from one individual to another.

RU refers to repeating unit, which is comprised of specific heteropolysaccharides synthesized by assembling individual monosaccharides into an oligosaccharide on an undecaprenyl phosphate (Und-P) carrier followed by polymerization into an oligosaccharide.

Signal sequence refers to a short (e,g, approximately 3-60 amino acids long) peptide at the N-terminal end of the protein that directs the protein to different locations.

“Polysaccharides” as used herein include saccharides comprising at least two monosaccharides. Polysaccharides include oligosaccharides, trisaccharides, repeating units comprising one or more monosaccharides (or monomers), and other saccharides recognized as polysaccharides by one of ordinary skill in the art. N-glycans are defined herein as mono-oligo- or polysaccharides of variable compositions that are linked to an ε-amide nitrogen of an asparagine residue in a protein via an N-glycosidic linkage.

Nucleic acids described herein include recombinant DNA and synthetic (e.g., chemically synthesized) DNA. Nucleic acids can be double-stranded or single-stranded. In the case of single-stranded nucleic acids, the nucleic acid can be a sense strand or antisense strand. Nucleic acids can be synthesized using oligonucleotide analogs or derivatives, as known to one of skill in the art in light of this specification.

The term “pharmaceutically acceptable carrier” refers to a carrier that is non-toxic. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the antibody. Such pharmaceutically acceptable carriers include, for example, liquid, semisolid, or solid diluents that serve as pharmaceutical vehicles, excipients, or media. Any diluent known in the art may be used. Exemplary diluents include, but are not limited to, polyoxyethylene sorbitan monolaurate, magnesium stearate, methyl- and propylhydroxybenzoate, talc, alginates, starches, lactose, sucrose, dextrose, sorbitol, mannitol, gum acacia, calcium phosphate, mineral oil, cocoa butter, and oil of theobroma.

The term “upstream process” is defined as the entire process from early cell isolation and cultivation, to cell banking and culture expansion of the cells until final harvest (termination of the culture and collection of the live cell batch). The upstream part of a bioprocess refers to the first step in which microbes/cells are grown, e.g. bacterial or mammalian cell lines, in bioreactors. Upstream processing involves all the steps related to inoculum development, media development, improvement of inoculum by genetic engineering process, optimization of growth kinetics so that product development can improve tremendously.

The term “downstream process” refers to the part where the cell mass from the upstream are processed to meet purity and quality requirements. Downstream processing is usually divided into three main sections: cell disruption, a purification section and a polishing section. The volatile products can be separated by distillation of the harvested culture without pre-treatment. Distillation is done at reduced pressure at continuous stills. At reduced pressure, distillation of product directly from fermentor may be possible.

“SDS-PAGE (sodium dodecyl sulfate polyacrylamide gel electrophoresis)” is a technique widely used in biochemistry and molecular biology to separate proteins according to their electrophoretic mobility (a function of length of polypeptide chain or molecular weight as well as higher order protein folding, posttranslational modifications and other factors). The resolution of this technique is such that it enables to distinguish proteins glycosylated to different degrees (e.g. mono-, di- or tri-glycosylated forms). After separation of Shigella-EPA bioconjugates by SDS-PAGE, the gel is stained with colloidal blue coomassie for detection. The ratio of the different glycoforms (degree of glycosylation) is subsequently determined over band pixel volumes by using a gel evaluation software e.g. Image Quant TL. Testing includes evaluation of several system suitability criteria as well as a product specific reference standard to assure proper assay performance.

“PgIL;” Oligosaccharyltransferases (OSTs or OTases) are membrane-embedded enzymes that transfer oligosaccharides from a lipid carrier to a nascent protein (unlike glycosyltransferases in the cytoplasm, which assemble oligosaccharides by sequential action, OTases transfer glycan to protein en bloc [5]). O-linked glycosylation consists of the covalent attachment of a sugar molecule (a glycan) to a side-chain hydroxyl group of an amino acid residue (e.g. serine, or threonine) in the protein target (e.g., pilin). Pilin-glycosylation gene L (PglL) proteins from, for example Neisseria meningitidis, are OTases involved in O-linked glycosylation. In the periplasm of gram-negative bacteria, PglLs transfer the glycan from UndPP-glycan to a pilin protein ( [3]). Unlike PglB (N-glycosylation), PglL does not require a 2-acetamido group at position C-2 of the reducing end or a β 1, 4 linkage between the first two sugars for activity and so is able to transfer virtually any glycan (Neisseria meningitidis PglL transfers, e.g., C. jejuni heptasaccharide, E. coli O7 antigen, E. coli K30 capsular structure, S. enterica O-antigen, and E. coli O16 peptidoglycan subunits to pilin in both E. coli and Salmonella cells) ( [3], [4], [37], [38]). NmPglL and homologues thereof, such as PglL from Neisseria gonorrhoeae (called “PglO”, [39] and [40]) and PilO from Pseudomonas aeruginosa ( [16]), are therefore substrate “promiscuous” (i.e., they have relaxed substrate specificity and so are able to transfer diverse oligo- and polysaccharides). [3] and [37] (per [4] and [38]). Neisseria meningitidis PglL (NmPgIL) Homologues are described herein (see Examples) and known to the art: [41], [42], [43]).

“PgIL OTase” herein encompasses Neisseria meningitidis PglL OTase as well as NmPglL OTase Homologues. Therefore, the term “PgIL OTases” herein includes, for example, Neisseria meningitidis PglL (NmPgIL) Oligosaccharyltransferase (OTase), Neisseria gonorrhoeae PglL (NgPglL) OTase, Neisseria lactamica 020-06 (NIPgIL) OTase, Neisseria lactamica ATCC 23970 PglL (Nl_{ATCC23970}PglL) OTase, and Neisseria gonorrhoeae F62 PglL (Ng_{F62}PglL) OTase.

“PglL Glycan Substrate”, “PglL Substrate” as used herein is a reference to a glycan which is transferrable by a PglL Otase (i.e., a glycan that is a substrate of PglL). See [3], [44], [45], [4], [46]. In certain embodiments, the PglL Glycan Substrate is attached to a lipid-carrier (“lipid-carrier-linked PglL Glycan Substrate”). In certain embodiments, the lipid-carrier is undecaprenol-pyrophosphate (UndPP), dolichol-pyrophosphate, or a synthetic equivalent thereof. In certain embodiments, the lipid-carrier is UndPP. In certain embodiments, the glycan is a “UndPP-linked PglL Substrate”. It is envisioned that a lipid-carrier-linked glycan is membrane-bound within a gram-negative host cell. A lipid-carrier-linked PglL Glycan Substrate being membrane bound may be said to be located “at the periplasm.” In certain embodiments, a NmPglL Glycan Substrate, a NgPglL Glycan Substrate, a N/PglL Glycan Substrate, or a NsPglL Glycan Substrate is specified. In certain embodiments, the PglL Glycan Substrate comprises a glycan having a reducing end of Glucose, Galactose, Galactofuranose, Rhamnose, GlcNAc, GalNAc, FucNAc, DATDH, GATDH, HexNAc, deoxy HexNAc, diNAcBac, or Pse. In certain embodiments the glycan is immunogenic (e.g., an “immunogenic PglL Glycan Substrate”). In certain embodiments the glycan is an O-antigen (e.g., a “PglL O-antigen Substrate”). See [3], [44], [45], [46], [47], [48].

Recombinant expression of a Neisserial PglL within a heterologous host cell is described herein and is known by the art (see [50], [51], [52], [53] (e.g., Table 1), [12], [3], [44], [7], [4], [49]; all incorporated herein by reference in their entireties).

### Abbreviations

TABLE 2

Abbreviations

| | |
|---|---|
| AltNAcA | 2-acetamido-2-deoxy-L-altruronic acid (Synonym: L-AltNAcA) |
| API | Active Pharmaceutical Ingredient |
| BSA | Albumin from bovine serum |
| CC | Complement Control |
| CFU | Colony forming unit |
| DD | Diarrhea disease |
| DNA | Deoxyribonucleic acid |
| DSP | Downstream-process |
| EDTA | Ethylenediaminetetraacetic acid |
| E. coli | Escherichia coli |
| ENG | Engineering batch during process scale up |
| EPA | Detoxified Pseudomonas aeruginosa exoprotein A |
| ETA | Wild-type Exotoxin A of Pseudomonas aeruginosa |
| ELISA | Enzyme linked immunosorbent assays |
| EU | Endotoxin units (Synonym: IU: International units) |
| FucNAc4N | 2-acetamido-4-amino-2, 4-dideoxy-D-fucose (Synonym: D-FucNAc4N) |
| GalA | D-Galacturonic acid (Synonym: D-GalA) |
| GalNAc | D-N-acetyl-galactosamine (Synonym: D-GalNAc) |
| Glc | D-Glucose (Synonym: D-Glc) |
| GlcNAc | D-N-acetyl-glucosamine (Synonym: D-GlcNAc) |
| GMP | Good manufacturing practice |
| GMT | Geometric mean titer |
| GMR | Geometric mean ratio |
| HPAEC-CD | High-performance anion-exchange chromatography with conductivity detection |
| HPAEC-PAD | High-performance anion-exchange chromatography with pulsed amperometric detection |
| i.m. | intramuscular |
| Leu | Leucine |
| LPS | Lipopolysaccharide |
| OD | Optical density |
| OD₆₀₀ | Optical density at 600 nm |
| PAGE | Polyacrylamide gel electrophoresis |
| PBS | Phosphate-buffered saline |
| PCR | Polymerase chain reaction |
| pgl | Protein Glycosylation |
| PglB | Oligosaccharyl transferase from Campilobacter jejuni |
| PglL | Oligosaccharyl transferase from Neisseria |
| pl | Isoelectric point |
| PS | Polysaccharide |
| Rha | L-Rhamnose (Synonym: L-Rha) |
| RS | Reference Solution |
| RU | Repeat Unit(s) |
| SBA | Serum Bactericidal Assay |
| SDS-PAGE | Sodium dodecylsulfate polyacrylamide gel electrophoresis |
| SEC | Size exclusion chromatography (preparative) |
| SE-HPLC | Size-exclusion HPLC |
| S-4V | Shigella4V |
| Sf2a | Shigella flexneri 2a |
| Sf2E | Shigella flexneri 2a- EPA bioconjugate (Synonyms: Sf2a-EPA) |
| Sf3a | Shigella flexneri 3a |
| Sf3E | Shigella flexneri 3a- EPA bioconjugate (Synonyms: Sf3a-EPA) |
| Sf6 | Shigella flexneri 6 |
| Sf6E | Shigella flexneri 6- EPA bioconjugate (Synonyms: Sf6-EPA) |
| Ss | Shigella sonnei |
| SsE | Shigella sonnei - EPA bioconjugate (Synonyms: Ss-EPA) |
| TFA | Trifluoroacetic |
| TMB | 3,3’,5,5’-Tetramethylbenzidine |
| TSA | Tryptic soy agar |
| UDP | Uridine diphosphate |
| USP | Upstream-process |
| UV 260/280 | Ratio of absorption at 260 nm over absorption at 280 nm |
| Val | Valine |
| VCC | Viable Cell Count |

### Introduction to Invention/ General Information

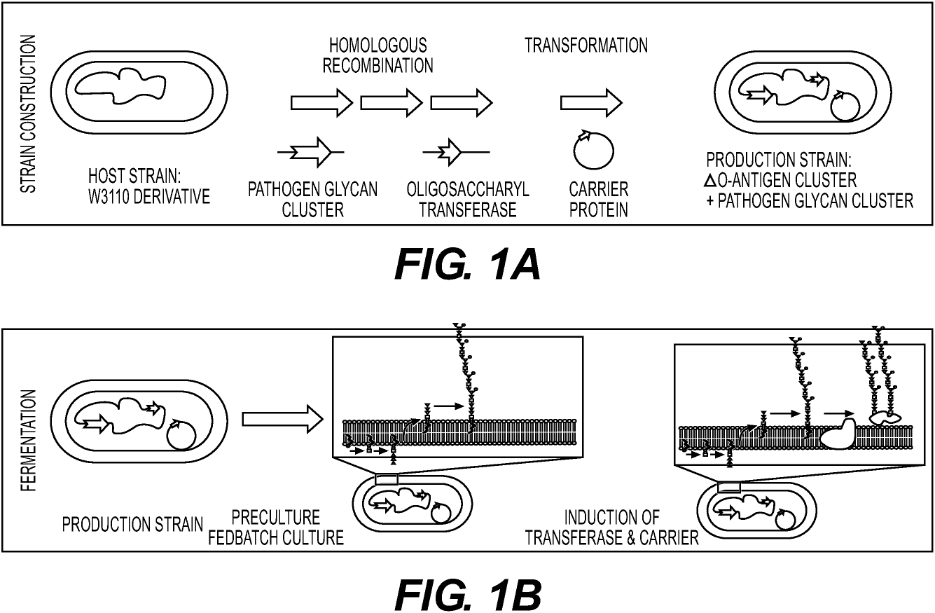

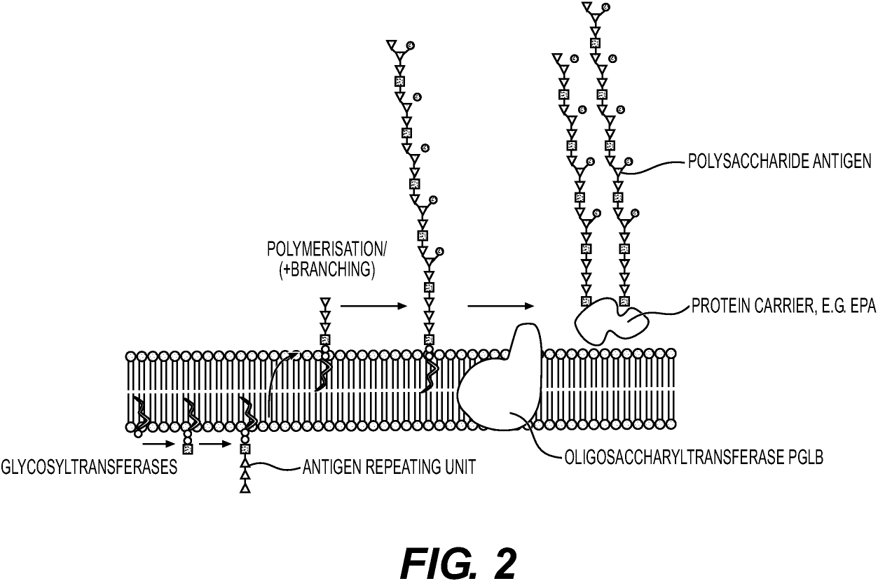

FIGS. 1A, 1B and 2 illustrate, a technology that enables the production of glycoconjugate vaccines directly synthesize in vivo using appropriately engineered bacterial cells. The technology is used for producing a bioconjugate based Shigella vaccine. In order that the production strain is able to produce the polysaccharide, the polysaccharide synthesizing enzymes of S. flexneri 2a, 3a, 6 and of S. sonnei were transferred into E. coli coexpressing the carrier protein EPA and an oligosaccharyltransferase. For Sf2E, Sf3E and Sf6E the oligosaccharyl transferase enzyme PglB, optionally from Campylobacter jejuni, is used to transfer the polysaccharide to a consensus sequence on the carrier protein detoxified Exotoxin A of Pseudomonas aeruginosa (EPA) in E. coli, resulting in a glycoprotein. For S. sonnei, PglL, optionally from Neisseria meningitidis or Neisseria gonorrhea is used to transfer the polysaccharide to a different consensus sequence on the carrier protein detoxified Exotoxin A of Pseudomonas aeruginosa (EPA) in E. coli, resulting in a glycoprotein.

The S-4V candidate vaccine is a tetravalent bioconjugate composed of O antigen-polysaccharides of S. sonnei and S. flexneri 2a, 3a and 6 conjugated to the recombinant Pseudomonas aeruginosa Exoprotein A, rEPA.

### Structure

The Immunogenic Composition of the tetravalent Shigella bioconjugate vaccine are the O-antigen polysaccharide chains from S. flexneri 2a, S. flexneri 3a, S. flexneri 6 and S. sonnei covalently linked to a detoxified protein carrier EPA.

For Sf2E, Sf3E and Sf6E the polysaccharide is linked covalently via the reducing end of the O-antigen to the side chain nitrogen atom of an asparagine residue residing in a consensus sequence for N-glycosylation (see Table 3). The signal peptide (underlined letters) is cleaved off during translocation to the periplasm. The N-glycosylation consensus sites are marked with bold letters. The Leu-Glu to Val mutation (italicized) leads to a significant detoxification of EPA.

TABLE 3

Modified detoxified Pseudomonas aeruginosa exotoxin A (EPA) protein carrier used for Sf2E, Sf3E and Sf6E.

| | | | | |
|---|---|---|---|---|
| Parameter | Value | | | |
| Amino acids | 630 (649 with signal peptide) | | | |
| Theoretical MW | 68.6 kDa (unglycosylated) | | | |
| Theoretical pl | 5.44 | | | |
| Theoretical extinction coefficient | 1.338 L×g⁻¹×cm⁻¹ | | | |
| Number of N-glycosylation sites | 3 (of which up to 3 can be glycosylated) | | | |
| Amino-acid sequence SEQ ID NO: 1 | MKKIWLALAG | LVLAFSASAA | EEAFDLWNEC | AKACVLDLKD |
| | GVRSSRMSVD | PAIADTNGQG | VLHYSMVLEG | GNDALKLAID |
| | NALSITSDGL | TIRLEGGVEP | NKPVRYSYTR | QARGSWSLNW |
| | LVPIGHEKPS | NIKVFIHELN | AGNQLSHMSP | IYTIEMGDEL |
| | LAKLARDATF | FVRAHESNEM | QPTLAISHAG | VSVVMAQAQP |
| | RREKRWSEWA | SGKVLCLLDP | LDGVYNKDQN | ATKLAQQRCN |
| | LDDTWEGKIY | RVLAGNPAKH | DLDIKPTVIS | HRLHFPEGGS |
| | LAALTAHQAC | HLPLEAFTKD | QNATKHRQPR | GWEQLEQCGY |
| | PVQRLVALYL | AARLSWNQVD | QVIRNALASP | GSGGDLGEAI |
| | REQPEQARLA | LTLAAAESER | FVRQGTGNDE | AGAASADVVS |
| | LTCPVAAGEC | AGPADSGDAL | LERNYPTGAE | FLGDGGDVSF |
| | STRGTQNWTV | ERLLQAHRQL | EERGYVFVGY | HGTFLEAAQS |
| | IVFGGVRARS | QDLDAIWRGF | YIAGDPALAY | GYAQDQEPDA |
| | RGRIRNGALL | RVYVPRWSLP | GFYRTGLTLK | DQNATKAPEA |
| | AGEVERLIGH | PLPLRLDAIT | GPEEEGGRVT | ILGWPLAERT |
| | VVIPSAIPTD | PRNVGGDLDP | SSIPDKEQAI | SALPDYASQP |

GKPPREDLK

For SsE, the polysaccharide is linked covalently via the reducing end of the O-antigen to the side chain oxygen atom of a serine residue residing in the O-glycosylation site (see Table 4). The signal peptide (underlined letters) is cleaved off during translocation to the periplasm. The O-glycosylation consensus site is bold with the putative O-glycosylated Serine in underlined. The Leu-Glu to Val mutation (italicized) leads to a significant detoxification of EPA.

TABLE 4

Modified detoxified Pseudomonas aeruginosa exotoxin A (EPA) protein carrier used for SsE

| | | | | |
|---|---|---|---|---|
| Parameter | Value | | | |
| Amino acids | 631 (650 with signal peptide) | | | |
| Theoretical MW | 68.6 kDa (unglycosylated) | | | |
| Theoretical pl | 5.33 | | | |
| Theoretical extinction coefficient | 1.440 L×g⁻¹×cm⁻¹ | | | |
| Number of O-glycosylation site | 1 | | | |
| Amino-acid sequence SEQ ID NO: 2 | MKKIWLALAG | LVLAFSASAA | EEAFDLWNEC | AKACVLDLKD |
| | GVRSSRMSVD | PAIADTNGQG | VLHYSMVLEG | GNDALKLAID |
| | NALSITSDGL | TIRLEGGVEP | NKPVRYSYTR | QARGSWSLNW |
| | LVPIGHEKPS | NIKVFIHELN | AGNQLSHMSP | IYTIEMGDEL |
| | LAKLARDATF | FVRAHESNEM | QPTLAISHAG | VSVVMAQAQP |
| | RREKRWSEWA | SGKVLCLLDP | LDGVYNYLAQ | QRCNLDDTWE |
| | GKIYRVLAGN | PAKHDLDIKP | TVISHRLHFP | EGGSLAALTA |
| | HQACHLPLEA | FTRHRQPRGW | EQLEQCGYPV | QRLVALYLAA |
| | RLSWNQVDQV | IRNALASPGS | GGDLGEAIRE | QPEQARLALT |
| | LAAAESERFV | RQGTGNDEAG | AASADVVSLT | CPVAAGECAG |
| | PADSGDALLE | RNYPTGAEFL | GDGGDVSFST | RGTQNWTVER |
| | LLQAHRQLEE | RGYVFVGYHG | TFLEAAQSIV | FGGVRARSQD |
| | LDAIWRGFYI | AGDPALAYGY | AQDQEPDARG | RIRNGALLRV |
| | YVPRWSLPGF | YRTGLTLGTW | PKDNTSAGVA | SSPTDIKAPE |
| | AAGEVERLIG | HPLPLRLDAI | TGPEEEGGRV | TILGWPLAER |
| | TVVIPSAIPT | DPRNVGGDLD | PSSIPDKEQA | ISALPDYASQ |

PGKPPREDLK

### Detoxified Pseudomonas Aeruginosa Exotoxin A (EPA) Protein Carrier

The wild-type Pseudomonas aeruginosa exotoxin A is a member of the ADP-ribosyltransferase toxin family comprising over 600 amino acid residues with a molecular mass of over 65 kDa.

The non-toxic (recombinant) mutant used for the Shigella4V vaccine candidate differs from wild-type toxin in at least two residues: Leu552 was changed to Val and Glu553 (in the catalytic domain) was deleted. Glu553 deletions were reported to significantly reduce toxicity and are not expected to be reversible. In addition to the detoxification mutation, glycosylation site consensus sequences were introduced (see Table 2 and Table 3).

### S. Flexneri 2a-Antigen Polysaccharide (PS)

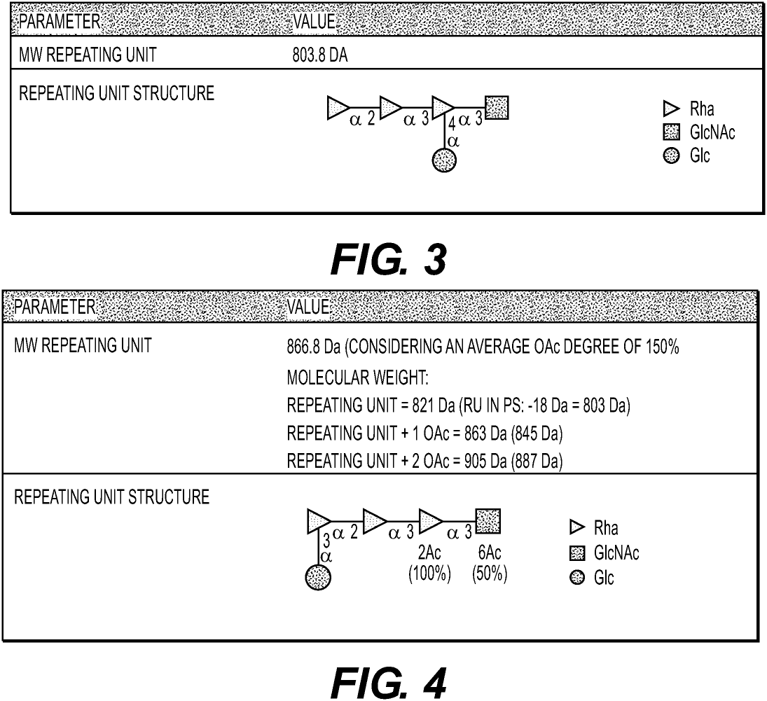

The S. flexneri 2a-antigen is composed of an average of approximately 16 repeating units (RU) and linked via the D-GlcNAc reducing end to the ε-nitrogen atom of an asparagine residue of one of the N-glycosylation consensus sites. The individual repeating units are linked via a β-1,2 linkage. The RU structure present on the bioconjugate has been resolved and is presented in FIG. 3. It deviates from the natural epitope by the lack of O-acetylation. Non-stochiometric O-acetylation was reported for the O-antigen of S. flexneri 2a by Perepelov [54]. (O-acetyl groups are linked to GlcNAc at position 6 (~60%) and to Rha lll at position 3 and 4 (~60%/~25%) (14)) and Kubler [55]. (30 -60% at position 6 of GlcNAc and 30-50% at position 3 of Rha lll). The rationale for the chosen antigenic structure is i) studies with synthetic non-O-acetylated oligosaccharides identified the branching glucose as an important epitope and were found to be immunogenic, ii) chemical S. flexneri 2a bioconjugate vaccines tested in clinical trials likely lack the O-acetyl groups as a result of the rather harsh treatment, and iii) sera of animals immunized with non-O-acetylated oligosaccharide bioconjugate recognize LPS extracted from S.flexneri 2a wild-type strains and show serum bactericidal activity.

The naturally occurring variation in PS chain length results in bioconjugates with varying molecular mass that can be resolved and analyzed using appropriate methods.

### S. Flexneri 3a-Antigen Polysaccharide (PS)

The S. flexneri 3a-antigen is composed of an average of approximately 16 RU’s and linked via the D-GlcNAc reducing end to the ε-nitrogen atom of an asparagine residue of one of the N-glycosylation consensus sites. Full proton and carbon assignments have been published for the O-acetylated RU of Shigella flexneri 3a, suggesting fully O-acetylated Rhamnose at position 1 and approx. 40% acetylation on the GlcNAc (FIG. 4) and is considered to be serotype determining. The individual repeating units are linked via a β-1,2 linkage. The strain has been engineered to represent the wild-type O-acetylation pattern and the RU structure present on the bioconjugate was resolved by nuclear magnetic resonance (NMR). The analysis confirmed 100% O-acetylation of Rhamnose at position 1 and approx. 50% acetylation on the GlcNAc.

### S. Flexneri 6-Antigen Polysaccharide (PS)

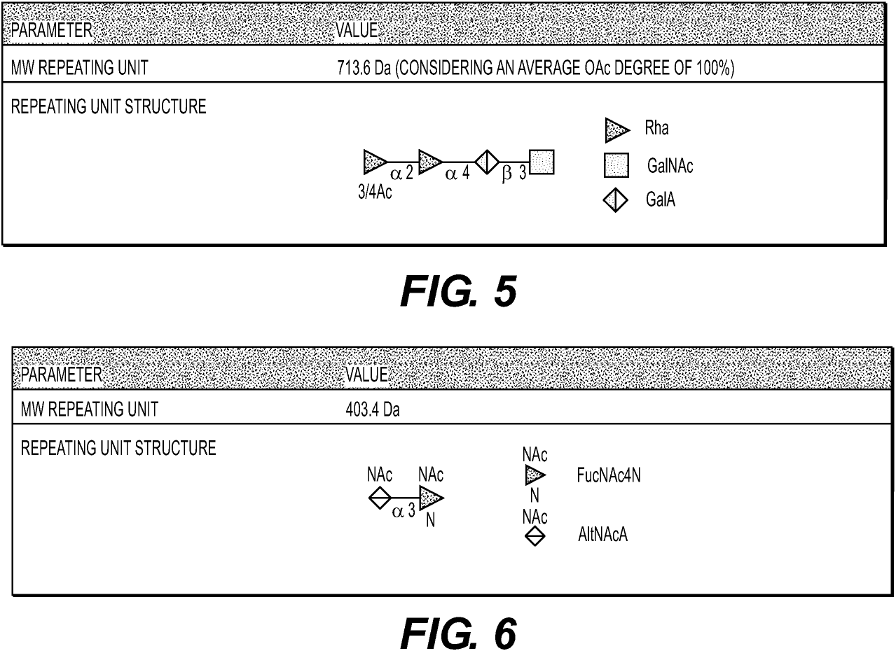

The S. flexneri 6-antigen is composed of an average of approximately 14 RU’s and linked via the D-GalNAc reducing end to the ε-nitrogen atom of an asparagine residue of one of the N-glycosylation consensus sites. The individual repeating units are linked via a β-1,2 linkage. Full proton and carbon assignments have been published for the O-acetylated RU of Shigella flexneri 6 [56], as well as the terminal RU attached to the core FIG. 5, indicating approx. 60% OAc occupation at position 3 and approx. 30% OAc at position 4 of Rha 3. The strain has been engineered to represent the wild-type structure and O-acetylation pattern and the RU structure present on the bioconjugate as been confirmed by nuclear magnetic resonance (NMR).

### S. Sonnei-Antigen Polysaccharide (PS)

The S. sonnei-antigen is composed of an average of approximately 29 RU’s and linked via the D-FucNAc4N reducing end to the hydroxyl group of the Serine in the O-glycosylation consensus site. The individual repeating units are linked via a β-1,4 linkage. Full proton and carbon assignments have been published for the disaccharide RU of Shigella Sonnei, FIG. 6. The strain has been engineered to represent the wild-type structure and the RU structure present on the bioconjugate has been confirmed by nuclear magnetic resonance (NMR).

### Embodiments

### Embodiments of the Present Invention Include, but Are Not Limited To

1. A composition comprising an O-antigen polysaccharide chain from each of S. flexneri 2a (Sf2E), S. flexneri 3a (Sf3E), S. flexneri 6 (Sf6E), and S. sonnei (SsE); wherein the O-antigen polysaccharide chains from S. flexneri 2a (Sf2E), S. flexneri 3a (Sf3E), S. flexneri 6 (Sf6E) are separately covalently linked to a protein carrier that has been modified to contain a N- glycosylation consensus sequence; wherein the N-glycosylation consensus sequence is D/E-X-N-Z-S/T (SEQ ID NO: 31), wherein X and Z can be any amino acid except proline and optionally wherein PglB is used to transfer the polysaccharide to the N-glycosylation consensus sequence D/E-X-N-Z-S/T (SEQ ID NO: 31), wherein X and Z can be any amino acid except proline; wherein SsE is covalently linked to a protein carrier containing an O-glycosylation consensus sequence capable of being glycosylation by PgIL, optionally wherein PglL is used to transfer the polysaccharide to the consensus sequence for SsE, TWPKDNTSAGVASSPTDIK (SEQ ID NO: 29).

2. A protein carrier selected from the group consisting of cholera toxin b subunit (CTB), tetanus toxoid (TT), tetanus toxin C fragment (TTc), diphtheria toxoid (DT), CRM197, Pseudomonas aeruginosa exotoxin A (EPA), C. jejuni Acriflavine resistance protein A (CjAcrA), E. coli Acriflavine resistance protein A (EcAcrA), and Pseudomonas aeruginosa PcrV (PcrV).

3. The protein carrier of embodiment 2 is Pseudomonas aeruginosa exotoxin A (EPA).

4. The EPA of embodiment 3 is a non-toxic (recombinant) mutant; wherein the Leu552 residue of EPA is substituted with Val; wherein the Glu552 residue is deleted.

5. The protein carrier of embodiment 2 comprises three N-glycosylation consensus sequences; wherein the protein carrier is glycosylated at only one (Mono-), two (Di-), or at all three sites (Tri-glycosylated) simultaneously.

6. The polysaccharide of embodiment 1 wherein Sf2E, Sf3E, and Sf6E are linked covalently via the reducing end of the O-antigen to the side chain nitrogen atom of an asparagine residue; wherein, the asparagine residue resides in the D/E-X-N-Z-S/T (SEQ ID NO: 31) N-glycosylation consensus sequence.

7. The composition of embodiment 1, wherein the S. flexneri 2a, S. flexneri 3a, S. flexneri 6 antigens are linked via the D-GlcNAc reducing end to the ε-nitrogen atom of an asparagine residue of one of the N-glycosylation consensus sites.

8. The polysaccharide of SsE is linked covalently via the reducing end of the O-antigen; wherein the glycan has a reducing end structure of
o a reducing end structure of Glucose, Galactose, Galactofuranose, Rhamnose, GlcNAc, GalNAc, FucNAc, DATDH, GATDH, HexNAc, deoxy HexNAc, diNAcBac, or Pse;
o a reducing end structure of DATDH, GlcNAc, GalNAc, FucNAc, Galactose, or Glucose;
o a reducing end structure of GlcNAc, GalNAc, FucNAc, or Glucose; or
o a S-2 to S-1 reducing end structure of Galactose-β1,4-Glucose; Glucuronic acid-β1,4-glucose; N-acetyl-fucosamine-α1,3-N-acetyl-galactosamine; Galactose-β1,4-glucose; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine.

9. The immunogenic composition the polysaccharide of SsE is linked covalently via the reducing end of the O-antigen to the side chain serine residue.

10. In an immunogenic composition the serine residue resides in the O-glycosylation site.

11. In an immunogenic composition the S. flexneri 2a - antigen is composed of an average of approximately 16 repeat units.

12. In an immunogenic composition the repeat units are linked via a β-1,2-linkage.

13. A gram-negative host cell comprising an immunogenic composition comprising the O-antigen polysaccharide chains from S. flexneri 2a (Sf2E), S. flexneri 3a (Sf3E), S. flexneri 6 (Sf6E), and S. sonnei (SsE); wherein the O-antigen polysaccharide chains are covalently linked to a protein carrier that has been modified to contain the Consensus sequence for protein glycosylation, D/E-X-N-Z-S/T (SEQ ID NO: 31), wherein X and Z can be any amino acid except proline; wherein PglB is used to transfer the polysaccharide to the consensus sequence for Sf2E, Sf3E, and Sf6E; and wherein PglL is used to transfer the polysaccharide to the consensus sequence for SsE.

14. A gram-negative host cell which is not S. sonnei comprising, the O-antigen polysaccharide chain form S. sonnei (SsE). In an embodiment, the host cell is Neisseria, Salmonella, Shigella, Escherichia, Pseudomonas, or Yersinia cell; wherein the host cell is E. coli; wherein the E. coli is genetically modified.

15. The host cell comprises a plasmid encoding the carrier protein EPA, optionally comprising at least one O-glycosylation consensus sequence suitable for glycosylation by PglL, optionally comprising the amino acid sequence TWPKDNTSAGVASSPTDIK (SEQ ID NO: 29).

16. The host cell comprises a plasmid encoding the oligosaccharyltransferase PglL.

17. The polysaccharide biosynthesis (rfb) cluster in SEQ ID NO: 1 and SEQ ID NO: 2 is replaced by an O-polysaccharide cluster; wherein the O-antigen ligase waaL is deleted.

18. The host cell comprises a plasmid encoding the carrier protein EPA; wherein the host cell comprises a plasmid encoding the oligosaccharyltransferase PglB (SEQ ID NO: 1) or PglL (SEQ ID NO: 2) wherein the araBAD genes required for arabinose metabolism is deleted; wherein the E. coli O16 glycotransferase gtrS is replaced with S. flexneri 2a glycotransferase gtrll; wherein the gtrll gene is replaced with gtrX from s. flexneri 3a; wherein the yeaS gene is replaced with OAcA; wherein the yahL gene is replaced with OAcD.

19. A method of producing a tetravalent bioconjugate vaccine, comprising the O-antigen polysaccharide chains from S. flexneri 2a (Sf2E), S. flexneri 3a (Sf3E), S. flexneri 6 (Sf6E), and S. sonnei (SsE); comprising the steps of a) culturing four separate host cells (optionally E. coli host cells) engineered to produce bioconjugates under conditions suitable for the production of bioconjugate, b) purifying one bioconjugate selected from the group consisting of Sf2E-EPA, Sf3E-EPA, Sf6E-EPA and SsE-EPA from each culture and c) mixing the Sf2E-EPA, Sf3E-EPA, Sf6EEPA and SsE-EPA bioconjugates, optionally at a ratio of 1:1:1:1; wherein the Campylobacter jejuni enzyme (PgIB) transfers the polysaccharide to a consensus sequence on the carrier protein detoxified Exotoxin A of Pseudomonas aeruginosa (EPA) in E. coli for the bioconjugates Sf2E, Sf3E, and Sf6E; wherein the method the PgIL, (optionally of Neisseria gonorrhea) transfers the polysaccharide to consensus sequence on the carrier protein detoxified Exotoxin A of Pseudomonas aeruginosa (EPA) in E. coli for the bioconjugate SsE.

20. The method of embodiment 21 wherein the host strain of Sf2E was genetically modified by replacing the polysaccharide biosynthesis (rfb) cluster with S. flexneri 2a O-polysaccharide cluster, deletion of the O-antigen ligase waaL, deletion of the araBAD genes required for arabinose metabolism, and replacement of the E. coli O16 glycosyltransferase gtrS with S. flexneri 2a glycosyltransferase gtrll.

21. The method of embodiment 21 wherein the host strain of Sf3E is genetically modified by the replacing the polysaccharide biosynthesis (rfb) cluster with S. flexneri 2a specific O-polysaccharide cluster, deletion of the O-antigen ligase waaL, deletion of araBAD genes required for arabinose metabolism, and replacement of the E. coli O16 glycosyltransferase gtrS with S. flexneri 2a glycosyltransferase gtrll.

22. The method of embodiment 21 wherein, the S. flexneri 2a glycosyltransferase gtrll is replaced with S. flexneri 3a glycosyltransferase gtrX; wherein the yeaS gene is replaced with the O-acetyltransferase OAcA gene; wherein the yahL gene is replaced with O-acetyltransferase OAcD gene; wherein the host strain of SsE was genetically modified by replacing the O16 O-polysaccharide biosynthesis (rfb) cluster with the Plesiomonas shigelloides O17, deletion of the wecA-wzzE, replacing O-antigen waaL with O-oligosaccharyltransferase PglL of N. gonorrhoeae, and replacing E. coli O16wzz polysaccharide chain length modulator with wzzB polysaccharide chain length modulator of S. typhimurium LT2.

23. A modified EPA protein of the invention may be modified by substitution of leucine 552 to valine (L552V) with reference to the amino acid sequence of SEQ ID NO: 30 (or an equivalent position in an amino acid sequence at least 80%, 85%, 90%, 92%, 95%, 96%, 97%, 98% or 99% identical to SEQ ID NO: 30). The modified EPA protein of the invention, may be modified by deletion of glutamine 553 (ΔE553) with reference to the amino acid sequence of SEQ ID NO: 30 (or an equivalent position in an amino acid sequence at least 80%, 85%, 90%, 92%, 95%, 96%, 97%, 98% or 99% identical to SEQ ID NO: 30). Preferably, the modified EPA protein of the invention, is modified by substitution of leucine 552 to valine (L552V) and deletion of glutamine 553 (ΔE553) with reference to the amino acid sequence of SEQ ID NO: 30 (or an equivalent position in an amino acid sequence at least 80%, 85%, 90%, 92%, 95%, 96%, 97%, 98% or 99% identical to SEQ ID NO: 30); wherein the term “modified EPA protein” refers to a EPA amino acid sequence (for example, having a amino acid sequence of SEQ ID NO: 30 or an amino acid sequence at least 80%, 85%, 90%, 91%, 92%, 93%, 94%, 95%, 96%, 97%, 98% or 99% identical to SEQ ID NO: 30), which EPA amino acid sequence has been modified by the addition, substitution or deletion of one or more amino acids (for example, by addition of a consensus sequence(s) selected from D/E-X-N-Z-S/T (SEQ ID NO: 31) and K-D/E-X-N-Z-S/T-K (SEQ ID NO: 32; or by substitution of one or more amino acids by a consensus sequence(s) selected from D/E-X-N-Z-S/T (SEQ ID NO: 31) and K-D/E-X-N-Z-S/T-K (SEQ ID NO: 32)). As used herein, in consensus sequences of the present invention X and Z are independently any amino acid apart from proline; preferably, X is Q (glutamine) and Z is A (alanine). The modified EPA protein may also comprise further modifications (additions, substitutions, deletions). In an embodiment, the modified EPA protein of the invention is a non-naturally occurring EPA protein (i.e. not native).

24. A modified EPA (Exotoxin A of Pseudomonas aeruginosa) protein having an amino acid sequence of SEQ ID NO: 30 or an amino acid sequence at least 80%, 85%, 90%, 92%, 95%, 96%, 97%, 98% or 99% identical to SEQ ID NO: 30, modified in that the amino acid sequence comprises one (or more) consensus sequence(s) selected from: D/E-X-N-Z-S/T (SEQ ID NO: 31) and K-D/E-X-N-Z-S/T-K (SEQ ID NO: 32), wherein the one (or more) consensus sequences have each been added next to, or substituted for one or more amino acids selected from specific amino acid residues within the EPA protein (consensus sequence sites); wherein the consensus sequence sites are selected from (i) one or more amino acids between amino acid residues 198-218 (e.g. one or more amino acids between amino acid residues 203-213, e.g. amino acid residue Y208), (ii) one or more amino acids between amino acid residues 264-284 (e.g. one or more amino acids between amino acid residues 269-279, e.g. amino acid residue R274), (iii) one or more amino acids between amino acid residues 308-328 (e.g. one or more amino acids between amino acid residues 313-323, e.g. amino acid residue S318), and (iv) one or more amino acids between amino acid residues 509-529 (e.g. one or more amino acids between amino acid residues 514-524; e.g. amino acid residue A519) of SEQ ID NO: 1 or at an equivalent position within an amino acid sequence at least 80%, 85%, 90%, 92%, 95%, 96%, 97%, 98% or 99% identical to SEQ ID NO: 30.

25. Native EPA is known to consist of three distinct structural domains [73]:
◯ Domain I, is an antiparallel β-structure. It includes residues 1-252 and residues 365-404. It has 17 β-strands. The first 13 strands form the structural core of an elongated β-barrel. Following strand 13 of domain I, the peptide chain traverses one face of the barrel, leading into the second domain.
◯ Domain II (residues 253-364) is composed of six consecutive a-helices with one disulfide linking helix A and helix B. Helices B and E are approximately 30 Å in length; helices C and D are approximately 15 Å long.
◯ Domain III is comprised of the carboxyl-terminal third of the molecule, residues 405-613. The most notable structural feature of domain III is its extended cleft. The domain has a less regular secondary structure than domains I and II.

26. An immunogenic fragment of EPA protein of the invention may be generated by removing and/or modifying one or more of these domains.

27. The immunogenic fragment of SEQ ID NO: 30 may comprise the amino acid residues of Domain l (residues 1-252 and residues 365-404) of SEQ ID NO: 30; the immunogenic fragment of SEQ ID NO: 30 may comprise the amino acid residues of Domain II (residues 253-364) of SEQ ID NO: 30; the immunogenic fragment of SEQ ID NO: 30 may comprise at least the amino acid residues of Domain III (residues 405-612) of SEQ ID NO: 30; the immunogenic fragment of SEQ ID NO: 30 may comprise the amino acid residues of Domain l (residues 1-252 and residues 365-404) of SEQ ID NO: 30 and Domain II (residues 253-364) of SEQ ID NO: 30; the immunogenic fragment of SEQ ID NO: 30 may comprise at least the amino acid residues of Domain II (residues 253-364) of SEQ ID NO: 30 and Domain III (residues 405-612) of SEQ ID NO: 30.

28. The amino acid numbers referred to herein correspond to the amino acids in SEQ ID NO: 30 and as described above, a person skilled in the art can determine equivalent amino acid positions in an amino acid sequence at least 80%, 85%, 90%, 92%, 95%, 96%, 97%, 98% or 99% identical to SEQ ID NO: 30 by alignment. The addition or deletion of amino acids from the variant and/or fragment of SEQ ID NO: 30 could lead to a difference in the actual amino acid position of the consensus sequence in the mutated sequence, however, by lining the mutated sequence up with the reference sequence, the amino acid in in an equivalent position to the corresponding amino acid in the reference sequence can be identified and hence the appropriate position for addition or substitution of the consensus sequence can be established.

29. The modified EPA protein of the invention may be an isolated modified EPA protein. The modified EPA protein of the invention may be a recombinant modified EPA protein. The modified EPA protein of the invention may be an isolated recombinant modified EPA protein.

30. The conjugate comprises a conjugate (e.g. bioconjugate) comprising (or consisting of) a modified EPA protein of the invention covalently linked to an antigen (e.g. a saccharide antigen, optionally a bacterial polysaccharide antigen), wherein the antigen is linked (either directly or through a linker).

31. The antigen is directly linked to the modified EPA protein of the invention.

32. The antigen is directly linked to an amino acid residue of the modified EPA protein.

33. A host cell comprising: one or more nucleotide sequences that encode polysaccharide synthesis genes, optionally for producing a bacterial polysaccharide antigen (e.g. an O-antigen from a Gram positive bacterium optionally from Shigella dysenteriae, Shigella flexneri, Shigella sonnei, Pseudomonas aeruginosa, Klebsiella pneumoniae, or a capsular polysaccharide from a Gram positive bacterium optionally from Streptococcus pneumoniae or Staphylcoccus aureus) or a yeast polysaccharide antigen or a mammalian polysaccharide antigen, optionally integrated into the host cell genome;a nucleotide sequence encoding a heterologous oligosaccharyl transferase, optionally within a plasmid; a nucleotide sequence that encodes a modified EPA protein of the invention, optionally within a plasmid.

34. Host cells may be modified to delete or modify genes in the host cell genetic background (genome) that compete or interfere with the synthesis of the polysaccharide of interest (e.g. compete or interfere with one or more heterologous polysaccharide synthesis genes that are recombinantly introduced into the host cell). These genes can be deleted or modified in the host cell background (genome) in a manner that makes them inactive/dysfunctional (i.e. the host cell nucleotide sequences that are deleted/modified do not encode a functional protein or do not encode a protein whatsoever). In an embodiment, when nucleotide sequences are deleted from the genome of the host cells of the invention, they are replaced by a desirable sequence, e.g. a sequence that is useful for glycoprotein production. Exemplary genes that can be deleted in host cells (and, in some cases, replaced with other desired nucleic acid sequences) include genes of host cells involved in glycolipid biosynthesis, such as waaL [80], the O antigen cluster (rfb or wb), enterobacterial common antigen cluster (wec), the lipid A core biosynthesis cluster (waa), galactose cluster (gal), arabinose cluster (ara), colonic acid cluster (wc), capsular polysaccharide cluster, undecaprenol-pyrophosphate biosynthesis genes (e.g. uppS (Undecaprenyl pyrophosphate synthase), uppP (Undecaprenyl diphosphatase)), Und-P recycling genes, metabolic enzymes involved in nucleotide activated sugar biosynthesis, enterobacterial common antigen cluster, and prophage O antigen modification clusters like the gtrABS cluster. In an embodiment, one or more of the waaL gene, gtrA gene, gtrB gene, gtrS gene, or a gene or genes from the wec cluster or a gene, or a gene or genes from the colonic acid cluster (wc), or a gene or genes from the rfb gene cluster are deleted or functionally inactivated from the genome of a prokaryotic host cell of the invention. In another embodiment, one or more of the waaL gene, gtrA gene, gtrB gene, gtrS gene, or a gene or genes from the wec cluster or a gene or genes from the rfb gene cluster are deleted or functionally inactivated from the genome of a prokaryotic host cell of the invention. In a specific embodiment the host cell of the invention is E. coli, wherein the native enterobacterial common antigen cluster (ECA, wec) with the exception of wecA, the colanic acid cluster (wca), and the O16-antigen cluster (wbb) have been deleted. In addition, the native lipopolysaccharide O-antigen ligase waaL may be deleted from the host cell of the invention. In addition, the native gtrA gene, gtrB gene and gtrS gene, may be deleted from the host cell of the invention.

35. A bioconjugate comprising a modified EPA protein of the invention linked to an antigen (e.g. a bacterial polysaccharide antigen). In a specific embodiment, said antigen is an O-antigen or a capsular polysaccharide. In an embodiment, the antigen is an O-antigen from a Gram negative bacterium. In an embodiment, the present invention provides a bioconjugate comprising a modified EPA protein of the invention linked to an antigen wherein the antigen is a saccharide, optionally a bacterial polysaccharide (e.g. from Shigella dysenteriae, Shigella flexneri, Shigella sonnei, Pseudomonas aeruginosa, Klebsiella pneumoniae, Streptococcus pneumoniae or Staphylcoccus aureus). The antigen is linked to an amino acid on the modified EPA protein selected from asparagine, aspartic acid, glutamic acid, lysine, cysteine, tyrosine, histidine, arginine or tryptophan (e.g. asparagine). Bioconjugates, as described herein, have advantageous properties over chemical conjugates of antigen-carrier protein, in that they require less chemicals in manufacture and are more consistent in terms of the final product generated.

36. A process for producing a bioconjugate that comprises (or consists of) a modified EPA protein linked to a saccharide, said process comprising (i) culturing the host cell of the invention under conditions suitable for the production of glycoproteins and (ii) isolating the bioconjugate produced by said host cell, optionally isolating the bioconjugate from a periplasmic extract from the host cell.

37. The composition (immunogenic) further comprising a buffer such as Tris (trimethamine), phosphate (e.g. sodium phosphate, sucrose phosphate glutamate), acetate, borate (e.g. sodium borate), citrate, glycine, histidine and succinate (e.g. sodium succinate), suitably sodium chloride, histidine, sodium phosphate or sodium succinate. In an embodiment, the buffer is sodium phosphate. In an embodiment, the pH is greater than 5.5. In an embodiment of the immunogenic composition the pH is 5.5 - 7.0. In an embodiment, the pH is 6.5. In an embodiment, the composition comprises salt. In an embodiment, the immunogenic composition comprises NaCl. In an embodiment, the composition comprises a non-ionic surfactant. In an embodiment, the composition comprises Polysorbate 80 (v/v). In an embodiment of the immunogenic composition the composition further comprises an adjuvant. In an embodiment of the immunogenic composition comprises the adjuvant, Aluminum hydroxide.

38. A method of immunizing against Shigellosis comprises a step of administering to a patient a dose of the immunogenic composition. In an embodiment of a method of immunizing against Shigellosis a dose comprises less than 20 µg, 0 - 50 µg, 40 - 50 µg, 0 - 20 µg, 0 - 10 µg, 0 - 6 µg, 10 - 20 µg, or 10 - 15 µg polysaccharide of each of the four Shigella O-antigens. In an embodiment a dose comprises 12 µg of each of the four antigens. In an embodiment a dose comprises 6 µg of each of the four antigens. In an embodiment a dose comprises 3 µg of each of the four antigens. In an embodiment a dose comprises 1 µg of each of the four antigens.

39. A method of immunizing against Shigellosis comprises administering to the mammal an immunologically effective amount of the immunogenic composition. In an embodiment a method comprises administering to a mammal an immunologically effective amount of the immunogenic composition.

40. A use of an immunogenic composition for inducing an antibody response in a mammal. In an embodiment comprising a use of the immunogenic composition for the manufacture of a medicament for inducing an antibody response in a mammal.

41. Each of the four bioconjugates is produced by a process starting with a specific cell substrate. In common to all four cell substrates is the original host strain E. coli W3110 [57], the replacement of the polysaccharide biosynthesis (rfb) cluster by the specific O-polysaccharide cluster, deletion of the O-antigen ligase waaL, introduction of a plasmid encoding a carrier protein, for example EPA and a plasmid encoding the oligosaccharyltransferase PglB or PgIL.

42. The modified carrier proteins can be used for bioconjugation. In certain embodiments, the modified carrier proteins can be used for in vivo bioconjugation within a gram-negative bacterial host cell. In certain embodiments, the modified carrier proteins can be used for conjugate production by incubating the modified carrier protein with a Neisserial PglL and a PglL glycan substrate, optionally in a suitable buffer.

43. O-glycosylated modified carrier proteins are produced using in vivo methods and systems. In certain embodiments, an O-glycosylated modified carrier protein (or bioconjugate) is made and then isolated from the periplasm of the host cell. In vivo conjugation (“bioconjugation”) of the present invention utilizes known methodologies for recombinant protein expression within a gram-negative bacterial cell and isolation therefrom, including sequence selection and optimization, vector design, cloning plasmids, culturing parameters, and periplasmic purification techniques. See, e.g., [58], [4], [7], [8], [9], [10], [11], [12], [3], [44], [6], [59], and [60]. Methods of producing bioconjugates using host cells are described in, for example, [61] and [62]. Bioconjugation offers advantages over in vitro chemical conjugation in that bioconjugation requires less chemicals for manufacture and is more consistent in terms of the final product generated.

44. Gram-negative bacterial cells for use with the present invention include, but are not limited to, a cell from the genera Neisseria, Shigella, Salmonella, Escherichia, Pseudomonas, Yersinia, Campylobacter, Vibrio, Klebsiella, or Helicobacter. In certain embodiments, the host cell is selected from the group consisting of Neisseria, Shigella, Salmonella, Escherichia, Pseudomonas, Yersinia, Campylobacter, and Helicobacter cells. In certain embodiments, the host cell is selected from the group consisting of Shigella, Salmonella, and Escherichia cells. In an embodiment, the gram-negative bacterial cell is classified as a Neisseria ssp., Shigella ssp., Salmonella ssp., Escherichia ssp, Pseudomonas ssp., Yersinia ssp., Campylobacter ssp., Vibrio ssp., Klebsiella ssp., or Helicobacter ssp. cell. The gram-negative bacterial host cell may be classified as a Neisserial ssp. cell other than Neisseria elongata. In a further embodiment, the gram-negative bacterial cell is a Shigella flexneri, Salmonella paratyphi, Salmonella enterica, E. coli, or Pseudomonas aeruginosa cell. In an embodiment, the host cell is selected from the group consisting of Shigella flexneri, Salmonella paratyphi, and Escherichia coli cells. In certain embodiments, the host cell is a Vibrio cholerae cell. In certain embodiments, the host cell is an Escherichia coli cell. In an embodiment, the gram-negative bacterial cell originated from E. coli strain K12, Top10, W3110, CLM24, BL21, SCM6 or SCM7. In certain embodiments, the host cell is a Shigella flexneri cell. In certain embodiments, the host cell is a Salmonella enterica cell. In an embodiment, the gram-negative bacterial cell originated from S. enterica strain SL3261, SL3749, SL326iδwaaL, or SL3749. In certain embodiments, the host cell is a Salmonella paratyphi cell. In certain embodiments, the host cell is a Pseudomonas aeruginosa cell. See [10], [9], [10], [63] at e.g. Table 1 and [12]; [6], [64], [7], [3], [44].

45. The gram-negative bacterial cell is modified such that the cell’s endogenous (periplasmic) O-antigen ligase (or “endogenous PglL homologue”) is reduced (deficient or “knockdown”) or knocked-out (KO) in expression or function as compared to control (e.g., wild type). In certain embodiments, “reduction of endogenous PglL homologue” or “the endogenous PglL homologue is reduced” is used to mean a reduction (e.g., a knockdown), which encompasses a knock-out, of the expression or function of the endogenous PglL homologue. In that way, a gram-negative bacterial cell of the present invention may be deficient in its endogenous PglL homologue. For example, the WaaL gene of E.coli and that of Salmonella enterica are functional homologues of N. meningitidis PglL ( [65], [66], and [62]). It is therefore envisioned that, for example, an Escherichia or Salmonella host cell for use with the present invention is modified such that the expression or function of WaaL is at least reduced as compared to a control (optionally wild type) Escherichia or Salmonella cell under essentially the same conditions. In certain embodiments, the host cell’s endogenous PglL gene (e.g., the waaL gene) has been replaced by a heterologous nucleotide sequence encoding an oligosaccharyltransferase. Techniques for knocking down or knocking out an endogenous PglL homologue are known and include, for example, mutation or deletion of the gene encoding the endogenous PglL homologue. See the Examples and, e.g., [4]; see also [67]. Gram-negative bacterial cells for use with the present invention include, but are not limited to, a cell from the genera Neisseria, Shigella, Salmonella, Escherichia, Pseudomonas, Yersinia, Campylobacter, Vibrio, Klebsiella, or Helicobacter. In certain embodiments, the host cell is selected from the group consisting of Neisseria, Shigella, Salmonella, Escherichia, Pseudomonas, Yersinia, Campylobacter, and Helicobacter cells. In certain embodiments, the host cell is selected from the group consisting of Shigella, Salmonella, and Escherichia cells. In an embodiment, the gram-negative bacterial cell is classified as a Neisseria ssp., Shigella ssp., Salmonella ssp., Escherichia ssp, Pseudomonas ssp., Yersinia ssp., Campylobacter ssp., Vibrio ssp., Klebsiella ssp., or Helicobacter ssp. cell. The gram-negative bacterial host cell may be classified as a Neisserial ssp. cell other than Neisseria elongata. In a further embodiment, the gram-negative bacterial cell is a Shigella flexneri, Salmonella paratyphi, Salmonella enterica, E. coli, or Pseudomonas aeruginosa cell. In an embodiment, the host cell is selected from the group consisting of Shigella flexneri, Salmonella paratyphi, and Escherichia coli cells. In certain embodiments, the host cell is a Vibrio cholerae cell. In certain embodiments, the host cell is an Escherichia coli cell. In an embodiment, the gram-negative bacterial cell originated from E. coli strain K12, Top10, W3110, CLM24, BL21, SCM6 or SCM7. In certain embodiments, the host cell is a Shigella flexneri cell. In certain embodiments, the host cell is a Salmonella enterica cell. In an embodiment, the gram-negative bacterial cell originated from S. enterica strain SL3261, SL3749, SL326iδwaaL, or SL3749. In certain embodiments, the host cell is a Salmonella paratyphi cell. In certain embodiments, the host cell is a Pseudomonas aeruginosa cell.

46. Gram-negative bacterial cells incorporating the glycosyltransferases, modified carrier proteins, PglL Otases, or PglL Glycan Substrates of this invention can be grown using various methods known in the art, for example, grown in a broth culture. The modified carrier proteins or O-glycosylated modified carrier proteins produced by the cells can be isolated using various methods known in the art, for example, lectin affinity chromatography ([3]).

47. An O-glycosylated modified carrier protein may be purified (to remove host cell impurities and unglycosylated carrier protein) and optionally characterized by techniques known in the art (see, e.g., [6], [68]; see also [11], [9], [69], [70], and [12]). Purification of a bioconjugate may be by cell lysis (including, e.g., one or more centrifugation steps) followed by one or more isolation steps (including, e.g., one or more chromatography steps or a combination of fractionation, differential solubility, centrifugation, and/or chromatography steps). Said one or more chromatographic steps may comprise ion exchange, anionic exchange, affinity, and/or sizing column chromatography, such as Ni2+ affinity chromatography and/or size exclusion chromatography. In a certain embodiment, one or more chromatographic steps comprises ion exchange chromatography. Therefore, one or more of the purified polypeptides may be operably linked to a tag (a purification tag). For example, affinity column IMAC (Immobilized metal ion affinity chromatography) may be used to bind the poly-histidine tag operably linked to the carrier protein, followed by anion exchange chromatography and size exclusion chromatography (SEC). For example, purification of a bioconjugate may be by osmotic shock extraction followed by anionic and/or size exclusion chromatography ([8]); or by osmotic shock extraction followed by Ni-NTA affinity and fluoroapatite chromatography ([6]).

48. Embodiments of the invention relates to the field of modified proteins, immunogenic compositions and vaccines comprising the modified proteins. Protein glycosylation is a common posttranslational modification in bacteria by which glycans are covalently attached to surface proteins, flagella, or pili, for example. [3]. Glycoproteins play roles in adhesion, stabilization of proteins against proteolysis, and evasion of the host immune response. [3]. Two protein glycosylation mechanisms are distinguished by the mode in which the glycans are transferred to proteins: one mechanism involves the transfer of carbohydrates directly from nucleotide-activated sugars to acceptor proteins (used in, e.g., protein O-glycosylation in the Golgi apparatus of eukaryotic cells and flagellin O-glycosylation in some bacteria). A second mechanism involves the preassembly of a polysaccharide onto a lipid-carrier (by glycosyltransferases) which is then transferred to a protein acceptor by an oligosaccharyltransferase (OTase). [3]. This second mechanism is used in, e.g., N-glycosylation in the endoplasmic reticulum of eukaryotic cells, the well-characterized N-linked glycosylation system of Campylobacter jejuni, and the more recently characterized O-linked glycosylation systems of Neisseria meningitidis, Neisseria gonococcus, and Pseudomonas aeruginosa. [3]. For O-linked glycosylation (O-glycosylation), glycans are generally attached to a serine or threonine residue on the protein acceptor. For N-linked glycosylation (N-glycosylation), glycans are generally attached to an asparagine residue on the protein acceptor. See generally [13].

49. The two best understood glycosylation systems are the C. jejuni N-linked glycosylation system and the Neisseria O-linked glycosylation system. [3], [4]. In these two systems, a polysaccharide (glycan donor) linked to an undecaprenyl pyrophosphate (UndPP) lipid-carrier is translocated (flipped) to the periplasm by a flippase. [5], [4]. In the periplasm, an oligosaccharyltransferase (OTase) transfers the glycan to a protein acceptor (pilin). [5], [4]. The OTase of C. jejuni (PgIB) transfers the glycan to the asparagine (N) in the conserved pilin pentapeptide motif D/E-X- N-Z-S/T (SEQ ID NO: 31) (where X and Z are any residues except proline). [6]. The OTase of N. meningitidis (NmPgIL) transfers the glycan to Ser63 in the N. meningitidis pilin PilE sequence (“sequon”) (N)-SAVTEYYLNHGEWPGNNTSAGVATSSElK-(C) (SEQ ID NO: 17, corresponding to residues 45-73 of mature N. meningitidis PilE sequence SEQ ID NO: 21). [3], [4], [7]. Until this disclosure, the pilin sequence onto which other OTases (from N. gonorrhoeae, N. lactamica, or N. shayeganii for example) transfer glycan was not known (see [39]).

50. Conjugate vaccines (comprising a carrier protein covalently linked to an immunogenic glycan) have been a successful approach for vaccination against a variety of bacterial infections. However, the chemical methods by which they are routinely produced are complex and comparatively inefficient ( [6] at FIG. 1). To increase conjugate vaccine production efficiency, in vivo methods (hence “bioconjugate vaccine”) have been in development. These in vivo methods leverage the N-glycosylation and O-glycosylation systems discussed above, particularly the OTase sequons, so that proteins which are not otherwise glycosylated by the OTase (carrier proteins), are glycosylated in vivo.

51. Carrier proteins AcrA and EPA were N-glycosylated in E. coli using heterologous polysaccharide as glycan donors and C. jejuni PglB because AcrA and EPA were first modified to incorporate an appropriate periplasmic signal sequence and at least one copy of the PglB sequon sequence D/E-X1- N-X2-S/T (O-linked glycosylation site). [6]; see also [8], [9], [10], [11], [12] (all of which are incorporated herein by reference in their entireties). The use of PglB-based bioconjugation production is limited because PglB only accepts certain sugar substrates: those containing an acetamido group at position C-2 of the reducing end and those that do not possess a β 1, 4 linkage between the first two sugars (i.e., the linkage between sugars “S-2” and “S-1”, the first sugar (S-1) comprising the reducing end and S-2 being adjacent to S-1). [4], [11], [14]. To overcome this limitation of PglB-based systems and because Neisserial PglLs are “promiscuous” with respect to sugar substrates ([3]), an O-glycosylation system using the PgIL OTase from Neisseria meningitidis has been the focus of recent work ([3], [15], [16], [17]; see also [18]).

52. Carrier proteins EPA, TTc, and CTB were O-glycosylated by N. meningitidis PglL in Shigella flexneri using polysaccharides which were endogenous to the Shigella flexneri host cell as glycan donors (“endogenous polysaccharide”) because each carrier protein was modified to incorporate a periplasmic signal sequence and one copy of the N. meningitidis PilE sequon sequence

53. Various methods can be used to analyze the glycans and conjugates of the invention including, for example, SDS-PAGE or capillary gel electrophoresis. O-antigen polymer length is defined by the number of repeat units that are linearly assembled. This means that the typical ladder like pattern is a consequence of different repeat unit numbers that compose the glycan. Thus, two bands next to each other in SDS PAGE (or other techniques that separate by size) differ by only a single repeat unit. These discrete differences are exploited when analyzing glycoproteins for glycan size: the unglycosylated carrier protein and the bioconjugate with different polymer chain lengths separate according to their electrophoretic mobilities. The first detectable repeat unit number (n1) and the average repeat unit number (n-average) present on a bioconjugate are measured. These parameters can be used to demonstrate batch to batch consistency or polysaccharide stability, for example.

54. A method of producing an O-glycosylated modified carrier protein, comprising culturing a gram-negative bacterial host cell, wherein the gram-negative bacterial host cell: (a) produces a Lipid-Carrier-Linked PgIL Glycan, (b) expresses a nucleotide sequence encoding a modified carrier protein, operatively linked to a polynucleotide sequence encoding a periplasmic signal sequence, and (c) expresses a nucleotide sequence encoding a PglL OTase, thereby producing an O-glycosylated modified carrier protein.

55. A method of producing an O-glycosylated modified carrier protein, comprising culturing a gram-negative bacterial host cell, wherein the gram-negative bacterial host cell: (a) expresses a nucleotide sequence encoding a PglL Glycan; (b) expresses one or more nucleotide sequence(s) encoding Glycosyltransferases capable of assembling a Lipid-Carrier-Linked PglL Glycan; (c) expresses a nucleotide sequence encoding a modified carrier protein, operatively linked to a polynucleotide sequence encoding a periplasmic signal sequence, and (d) expresses a nucleotide sequence encoding a PgIL OTase, thereby producing an O-glycosylated modified carrier protein.

56. The Lipid-Carrier-Linked PgIL Glycan is an O-antigen. In certain embodiments, the O-antigen is S. sonnei O-antigen.

57. A method of producing an O-glycosylated modified carrier protein, comprising culturing a gram-negative bacterial host cell, wherein the gram-negative bacterial host cell: (a) comprises lipid-Carrier-Linked PglL Glycan Substrate, (b) comprises in the periplasm a modified carrier protein, the modified carrier protein being characterized by a carrier protein comprising at least one O-linked glycosylation site, and(c) comprises a Neisseria PglL OTase. In certain embodiments, the Lipid-Carrier-Linked PglL Glycan Substrate comprises at the reducing end a Glucose, Galactose, Galactofuranose, Rhamnose, GlcNAc, GalNAc, FucNAc, DATDH, GATDH, HexNAc, deoxy HexNAc, diNAcBac, or Pse. In certain embodiments, the Lipid-Carrier-Linked PglL Glycan Substrate is endogenous to the host cell. In certain embodiments, the method further comprises isolating an O-glycosylated modified carrier protein from the cell.

58. The use of a composition comprising the O-antigen polysaccharide chains from S. flexneri 2a (Sf2E), S. flexneri 3a (Sf3E), S. flexneri 6 (Sf6E), and S. sonnei (SsE) for inducing an antibody response in a mammal. Certain embodiments comprise the use of a composition comprising the O-antigen polysaccharide chains from S. flexneri 2a (Sf2E), S. flexneri 3a (Sf3E), S. flexneri 6 (Sf6E), and S. sonnei (SsE) for inducing an immune response in a mammal. Certain embodiments comprise the use of a composition comprising the O-antigen polysaccharide chains from S. flexneri 2a (Sf2E), S. flexneri 3a (Sf3E), S. flexneri 6 (Sf6E), and S. sonnei (SsE) for the manufacture of a medicament for inducing an antibody response in a mammal. Certain embodiments comprise the use of a composition comprising the O-antigen polysaccharide chains from S. flexneri 2a (Sf2E), S. flexneri 3a (Sf3E), S. flexneri 6 (Sf6E), and S. sonnei (SsE) for the manufacture of a medicament for inducing an immune response in a mammal.

59. Bioconjugate technology is used for the manufacturing of a bioconjugate based Shigella vaccine. In order that the production strain is able to produce the polysaccharide, the polysaccharide-synthesizing enzymes of S. flexneri 2a, 3a, 6 and of S. sonnei were transferred into E. coli coexpressing the carrier protein EPA and an oligosaccharyltransferase. For Sf2E, Sf3E and Sf6E the Campylobacter jejuni enzyme (PgIB) is used to transfer the polysaccharide to a consensus sequence on the carrier protein detoxified Exotoxin A of Pseudomonas aeruginosa (EPA) in E. coli, resulting in a glycoprotein (FIG. 1 and FIG. 2). For Shigella sonnei the PgIB catalyzed transfer of the polysaccharide onto a carrier protein was not efficient. It thus was decided to use PglL of Neisseria gonorrhoeae which previously has been shown to transfer polysaccharides onto carrier proteins resulting in O-linked glycosylation.

60. The bioconjugate vaccine is expressed in the periplasm of E. coli, extracted and purified in a simplified process.

61. The compositions described herein are formulated to be suitable for the intended route of administration to a subject. For example, the compositions described herein may be formulated to be suitable for subcutaneous, parenteral, oral, intradermal, transdermal, colorectal, intraperitoneal, and rectal administration. In a specific embodiment, the pharmaceutical composition may be formulated for intravenous, oral, intraperitoneal, intranasal, intratracheal, subcutaneous, intramuscular, topical, intradermal, transdermal or pulmonary administration.

62. The compositions described herein additionally comprise one or more buffers, e.g., phosphate buffer and sucrose phosphate glutamate buffer. In other embodiments, the compositions described herein do not comprise buffers.

63. The compositions described herein additionally comprise one or more salts, e.g., sodium chloride, calcium chloride, sodium phosphate, monosodium glutamate, and aluminum salts (e.g., aluminum hydroxide, aluminum phosphate, alum (potassium aluminum sulfate), or a mixture of such aluminum salts). In other embodiments, the compositions described herein do not comprise salts

64. Pharmaceutically acceptable excipients can be selected by those of skill in the art. For example, a pharmaceutically acceptable excipient may be a buffer, such as Tris (trimethamine), phosphate (e.g. sodium phosphate, sucrose phosphate glutamate), acetate, borate (e.g. sodium borate), citrate, glycine, histidine and succinate (e.g. sodium succinate), suitably sodium chloride, histidine, sodium phosphate or sodium succinate. A pharmaceutically acceptable excipient may include a salt, for example sodium chloride, potassium chloride or magnesium chloride. Optionally, a pharmaceutically acceptable excipient contains at least one component that stabilizes solubility and/or stability. Examples of solubilizing/stabilizing agents include detergents, for example, laurel sarcosine and/or polysorbate (e.g. TWEEN 80 (Polysorbate-80)). Examples of stabilizing agents also include poloxamer (e.g. poloxamer 124, poloxamer 188, poloxamer 237, poloxamer 338 and poloxamer 407). A phamaceutically acceptable excipient may include a non-ionic surfactant, for example polyoxyethylene sorbitan fatty acid esters, TWEEN 80 (Polysorbate-80), TWEEN 60 (Polysorbate-60), TWEEN 40 (Polysorbate-40) and TWEEN 20 (Polysorbate-20), or polyoxyethylene alkyl ethers (suitably polysorbate-80). Alternative solubilizing/stabilizing agents include arginine, and glass forming polyols (such as sucrose, trehalose and the like). A pharmaceutically excipient may be a preservative, for example phenol, 2-phenoxyethanol, or thiomersal. Other pharmaceutically acceptable excipients include sugars (e.g. lactose, sucrose), and proteins (e.g. gelatine and albumin). Pharmaceutically acceptable excipients for use with the present invention include saline solutions, aqueous dextrose and glycerol solutions (also referred to as “carriers” or “fillers” in the art).

65. Immunogenic compositions if the invention may also comprise diluents such as saline, and glycerol. Additionally, immunogenic compositions may comprise auxiliary substances such as wetting agents, emulsifying agents, pH buffering substances, and/or polyols.

66. Immunogenic compositions if the invention may also comprise one or more salts, e.g. sodium chloride, calcium chloride, sodium phosphate, monosodium glutamate, and aluminum salts (e.g. aluminum hydroxide, aluminum phosphate, alum (potassium aluminum sulfate), or a mixture of such aluminum salts).

67. Immunogenic compositions or vaccines of the invention may be used to induce an immune or antibody response and/or protect or treat a mammal susceptible to infection, by administering said immunogenic composition or vaccine composition to said mammal via systemic or mucosal route. These administrations may include injection via the intramuscular (IM), intraperitoneal, intradermal (ID) or subcutaneous routes; or via mucosal administration to the oral/alimentary, respiratory, genitourinary tracts. For example, intranasal (IN) administration may be used. Although the immunogenic composition or vaccine of the invention may be administered as a single dose, components thereof may also be co-administered together at the same time or at different times. For co-administration, the optional adjuvant, for example, may be present in any or all of the different administrations, however in one particular aspect of the invention it is present in combination with the immunogenic O-glycosylated modified carrier protein. In addition to a single route of administration, two different routes of administration may be used. Following an initial vaccination, subjects may receive one or several booster immunizations adequately spaced.

### EXAMPLES

It is understood that the examples and embodiments described herein are for illustrative purposes only and that various modifications or changes in light thereof will be suggested to persons skilled in the art and are to be included within the spirit and purview of this application.

### Example 1: Generation of the Sf2E Cell Substrate E. Coli

For Sf2E the host strain was genetically modified by replacement of the polysaccharide biosynthesis (rfb) cluster by the S. flexneri 2a specific O-polysaccharide cluster, deletion of the O-antigen ligase waaL, deletion of the araBAD genes required for arabinose metabolism, exchange of the E. coli O16 glycosyltransferase gtrS with the S. flexneri 2a glycosyltransferase gtrll. The final host strain was transformed with a plasmid encoding the carrier protein EPA and a plasmid encoding the oligosaccharyltransferase PgIB.

The bacterial cell substrate for the production of Sf2E is a derivative of the Escherichia coli K-12 strain W3110, with the following modifications: full length chromosomal deletion of the O-antigen ligase gene waaL (ΔwaaL), chromosomal deletion of the araBAD genes required for arabinose metabolism, replacement of the gtrS gene by gtrll from S. flexneri 2a, replacement of the chromosomal rfb cluster by the rfb cluster of S. flexneri 2a (resulting in the genotype ΔrfbW3110::rfbCCUG29416), and introduction of pglB plasmid, and introduction of EPA plasmid.

### Example 2: Generation of the Sf3E Cell Substrate E. Coli

For Sf3E the host strain was genetically modified by the replacement of the polysaccharide biosynthesis (rfb) cluster with the S. flexneri 2a specific O-polysaccharide cluster, the deletion of the O-antigen ligase waaL, the deletion of the araBAD genes required for arabinose metabolism and the exchange of the E. coli O16 glycosyltransferase gtrS with the S. flexneri 2a glycosyltransferase gtrll which was later exchanged with the S. flexneri 3a glycosyltransferase gtrX. Further modification was performed by exchanging yeaS gene with the O-acetlytransferase OAcA gene and exchanging yahL gene with the O-acetyltransferase OAcD gene. The final host strain was transformed with a plasmid encoding the carrier protein EPA and a plasmid encoding the oligosaccharyltransferase PgIB and the O-acetyltransferase OAcD (second copy).

The bacterial cell substrate for the production of Sf3E is a derivative of the Escherichia coli K-12 strain W3110, with the following modifications: full length chromosomal deletion of the O-antigen ligase gene waaL (ΔwaaL), chromosomal deletion of the araBAD genes required for arabinose metabolism, replacement of the gtrS gene by gtrll from S. flexneri 2a, replacement of the chromosomal rfb cluster by the rfb cluster of S. flexneri 2a strain (resulting in the genotype ΔrfbW3110::rfbCCUG29416), replacement of the gtrll gene by gtrX from S. flexneri 3a, replacement of yeaS gene by OAcA, replacement of yahL gene by OAcD, introduction of PgIB plasmid and introduction of EPA plasmid.

### Example 3: Generation of the Sf6E Cell Substrate E. Coli

The Sf6E production strain was created by genetically modifying the host E. coli W3110. A part of the polysaccharide biosynthesis (rfb) cluster (rfbX-glf-rfc-wbbl-wbbJ-wbbK-wbbL_2-insH_8-wbbL_1) was replaced by a combination of polysaccharide biosynthesis genes required for the generation of the S.flexneri 6 O-antigen (S.flexneri 6 wzx-wzy-wfbY-wfbZ; E.coli O157:H45 UDP-galactose 4-epimerase Z3206 carrying a HAtag; E.coli W3110 UDP-glucose 6-dehydrogenase ugd; R. ornithinolytica UDP galacturonate 4-epimerase uge). The rfbW3110 cluster genes rmlB, rmID, rfbA and rfbC were retained in the host genome and used for biosynthesis of L-Rhamnose, a sugar required for synthesis of the S.flexneri 6 O-antigen. Further modifications comprised the deletion of the O-antigen ligase waaL, insertion of the codon usage optimized (cuo) E.coli O157:H45 UDP-galactose 4-epimerase Z3206cuo into waaL locus and the exchange of the yeaS gene by the S.flexneri 6 O-acetyltransferase C OAcC. The final host strain was transformed with a plasmid encoding the oligosaccharyltransferase PgIB and the carrier protein EPA and a plasmid harboring a second copy of the carrier protein EPA.

The bacterial cell substrate for the production of Sf6E is a derivative of the Escherichia coli K-12 strain W3110, with the following modifications: full length chromosomal deletion of the O-antigen ligase gene waaL (ΔwaaL), replacement of the chromosomal rfb cluster genes rfbX-glf-rfc-wbbl-wbbJ-wbbK-wbbL_2-insH_8-wbbL_1 by the S.flexneri 6 O-antigen building polysaccharide biosynthesis genes S. flexneri 6 wzx-wzy-wfbY-wfbZ; E.coli O157:H45 UDP-galactose 4-epimerase Z3206-HAtag; E.coli W3110 UDP-glucose 6-dehydrogenase ugd; R. ornithinolytica UDP galacturonate 4-epimerase uge, insertion of the UDP-galactose 4-epimerase Z3206cuo into waaL locus, replacement of the chromosomal yeaS gene by the S.flexneri 6 O-acetyltransferase C gene OAcC, introduction of pglB plasmid and introduction of EPA plasmid.

### Example 4: Generation of the SsE Cell Substrate E. Coli

For SsE the host strain was genetically modified by replacement of the O16 O-polysaccharide biosynthesis (rfb) cluster with the Plesiomonas shigelloides O17 (=S.sonnei) specific O-polysaccharide cluster (GenBank AF285970.1, nucleotides 1178-12270, lacking the native wzz polysaccharide chain length modulator function), deletion of wecA-wzzE which are components interfering with the recombinant S.sonnei O-antigen biosynthesis, exchange of the O-antigen ligase waaL with the O-oligosaccharyltransferse PglL of N.gonorrhoeae (Genbank CNT56492), and exchange of the E. coli O16wzz polysaccharide chain length modulator with the wzzB polysaccharide chain length modulator of S. typhimurium LT2 (Genbank NC_003197). The final host strain was transformed with a plasmid encoding the carrier protein EPA and a plasmid encoding one copy of the O-oligosaccharyltransferase PglL from N.gonorrhoeae and one copy of the polysaccharide chain length modulator wzzB from S.typhimurium LT2.

### Example 5

### Materials and Methods

Escherichia coli deficient in O-antigen lipopolysaccharide ligase gene waaL (E. coli W3110 ΔwaaL, ΔwecA-wzzE, AO16::wbgT-wbgZ cluster of P.shigelloides O17 (S.sonnei) (“E.coli W3110Δwaal” hereafter)) containing a chromosomal copy of a polysaccharide biosynthesis cluster (O-antigen or capsular polysaccharide) as well as two plasmids expressing PglL and a modified carrier protein was used. A single colony was inoculated in 50 ml TBdev medium [yeast extract 24 g/L, soy peptone 12 g/L, glycerol 100% 4.6 mL/L, K₂HPO₄ 12.5 g/L, KH₂PO₄ 2.3 g/L, MgCl₂x6H2O 2.03 g/L) and grown at 30° C. to an OD of 0.8. At this point, 0.1 mM IPTG and 0.1% arabinose were added as inducers. The culture was further incubated o/n and harvested for further analysis (see [00119]). In case of bioreactor evaluation, a 50 mL (uninduced) o/n culture was used to inoculate a 11 culture in a 21 bioreactor. The bioreactor was stirred with 500-1000 rpm, pH was kept at 7.2 by auto-controlled addition of either 2 M KOH or 20% H₃PO₄ and the cultivation temperature was set at 30° C. The level of dissolved oxygen (pO2) was kept at 10% oxygen. In batch phase cells were grown in a TBdev medium as described above but containing glycerol at 50 g/L. As feed medium TBdev supplemented with 250 g/L glycerol and 0.1% IPTG (one-plasmid system) or 0.1% IPTG and 2.5% arabinose (two-plasmid system) was used. Induction with 0.1 mM IPTG (one-plasmid system) and 0.1 mM IPTG and 0.1% arabinose (2-plasmid system) was done at OD=35, prior to starting the fed-batch phase of growth. A linear feed rate was sustained for 24h, followed by a 16h starvation period. The bioreactor culture was harvested after a total of ≈40 h cultivation, when it should have reached an OD600 of ±80.

The production process was analyzed by Coomassie brilliant blue staining or Western blot as described previously ([71]). After being blotted on nitrocellulose membrane, the sample was immunostained with the either anti-His, anti-glycan or anti-carrier-protein. Anti-rabbit IgG-HRP (Biorad) was used as secondary antibody. Detection was carried out with ECL™ Western Blotting Detection Reagents (Amersham Biosciences, Little Chalfont Buchinghamshire).

For periplasmic protein extraction, the cells were harvested by centrifugation for 20 min at 10,000 g and resuspended in 1 volume 0.9% NaCl. The cells were pelleted by centrifugation during 25-30 min at 7,000 g. The cells were resuspended in Suspension Buffer (25% Sucrose, 100 mM EDTA 200 mM Tris HCl pH 8.5, 250 OD/ml) and the suspension was incubated under stirring at 4-8° C. during 30 min. The suspension was centrifuged at 4-8° C. during 30 min at 7,000-10,000 g. The supernatant was discarded, the cells were resuspended in the same volume ice cold 20 mM Tris HCl pH 8.5 and incubated under stirring at 4-8° C. during 30 min. The spheroblasts were centrifuged at 4-8° C. during 25-30 min at 10,000 g, the supernatant was collected and passed through a 0.2 g membrane. Periplasmic extract was loaded on a 7.5% SDS-PAGE, and stained with Coomasie for identification.

For bioconjugate purification, the supernatant containing periplasmic proteins obtained from 100,000 OD of cells was loaded on a Source Q anionic exchange column (XK 26/40≈180 ml bed material) equilibrated with buffer A (20 mM Tris HCl pH 8.0). After washing with 5 column volumes (CV) buffer A, the proteins were eluted with a linear gradient of 15CV to 50% buffer B (20 mM Tris HCl+1M NaCl pH 8.0) and then 2CV to 100% buffer B. Protein were analyzed by SDS-PAGE and stained by Coomassie. Bioconjugate may elute at conductivity between 6-17 mS. The sample was concentrated 10 times and the buffer was exchanged to 20 mM Tris HCl pH 8.0.

Bioconjugate was loaded on a Source Q column (XK 16/20≈28 ml bed material) equilibrated with buffer A: 20 mM Tris HCl pH 8.0. The identical gradient that was used above was used to elute the bioconjugate. Protein were analyzed by SDS-PAGE and stained by Coomassie. Normally the bioconjugate elutes at conductivity between 6-17 mS. The sample was concentrated 10 times and the buffer was exchanged to 20 mM Tris HCl pH 8.0.

Bioconjugate was loaded on Superdex 200 (Hi Load 26/60, prep grade) that was equilibrated with 20 mM Tris HCl pH 8.0. Protein fractions from Superdex 200 column were analyzed by SDS-PAGE and stained by Coomassie stained.

Bioconjugates from different purification steps were analyzed by SDS-PAGE and stained by Coomassie. Bioconjugate is purified to more than 98% purity using the process. Bioconjugate can be successfully produced using this technology.

### Carrier Protein Optimization

Pseudomonas exotoxin A (EPA) carrier protein (SEQ ID NO: 12) was modified to incorporate one or more O-linked glycosylation site s from Neisseria meningitidis pilin PilE (wild type sequence provided as SEQ ID NO: 20) (for methods see [29]; [6]; [6]; and [31], all incorporated herein by reference in their entireties). Recombinant EPA (rEPA, SEQ ID NO: 12) was modified to make three other recombinant EPA proteins:
the first having been modified to incorporate, at its N-terminus, the NmPilE O-linked glycosylation site SEQ ID NO: 20 (corresponding to residues 45-73 of SEQ ID NO: 21); twenty-nine (29) amino acid long) (rEPA1, SEQ ID NO: 37).
The second having been modified to incorporate, at internal residue A375 with respect to SEQ ID NO: 12 0, the NmPilE O-linked glycosylation site SEQ ID NO: 20 (rEPA2, SEQ ID NO: 53).
The third having been modified to incorporate, at its C-terminus, the NmPilE O-linked glycosylation site SEQ ID NO: 20 (rEPA3, SEQ ID NO: 38).

### Example 6: Degree of Glycosylation Analysis

The carrier protein EPA for used for serotypes Sf2E, Sf3E and Sf6E harbours three glycosylation sites. The protein thus may be glycosylated at only one (Mono-), two (Di-) or at all three sites (Tri-glycosylated) at the same time. To characterize the distribution of the different glycosylation forms, the ENG (scale-up) and GMP API batches of Sf2E, Sf3E and Sf6E were analyzed by a high-resolution SDS-PAGE based method. SsE bioconjugates were not analysed by this method since the EPA carrier protein used for Shigella sonnei only contains one glycosite, accordingly only monoglycosylated forms are possible.

Glycoform bands were integrated and relative intensities were calculated to express the degree of glycosylation. All SDS-PAGE analyses for characterization were performed by the CMO as supportive data for batch release.

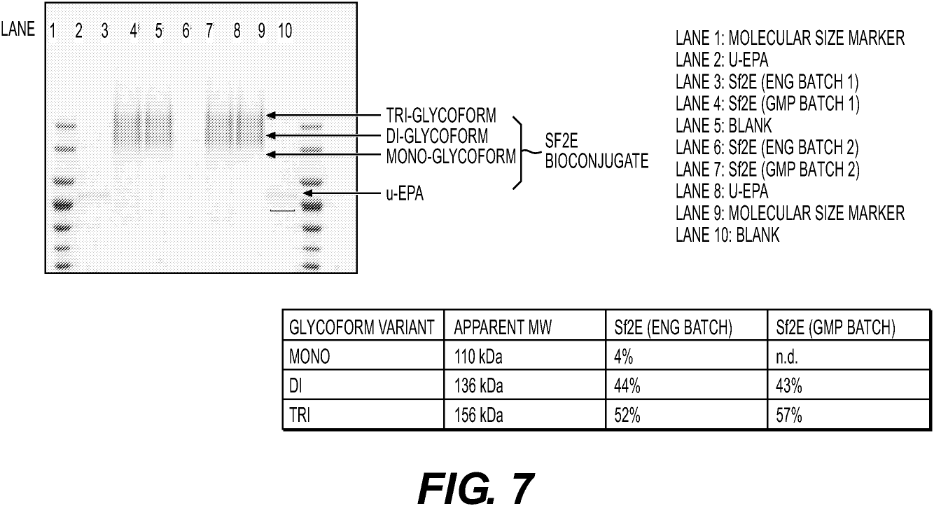

Sf2E ENG and GMP API batches showed a nearly identical degree of glycosylation with predominantly di- and tri-glycosylated forms, demonstrating the comparability of the two batches (FIG. 7).

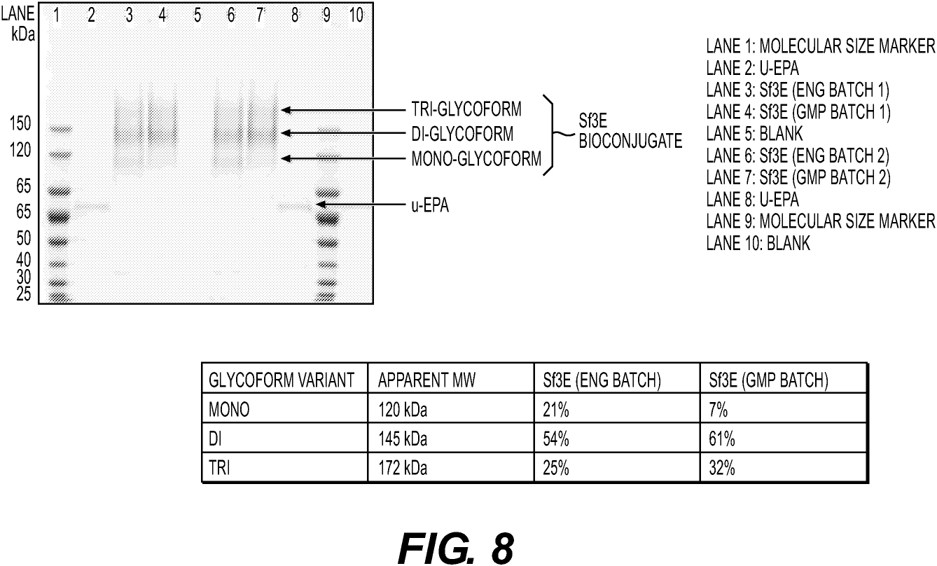

Sf3E ENG and GMP API batches showed a slightly different degree of glycosylation, whereat the main difference can be observed for the amount of the mono-glycosylated form (FIG. 8). The main reason for this glycoform distribution difference, especially the decrease of monoglycosylated form, is attributed to adapted DSP pooling criteria for the Sf3E GMP API batch. Most predominant in both batches is the di-glycosylated form.

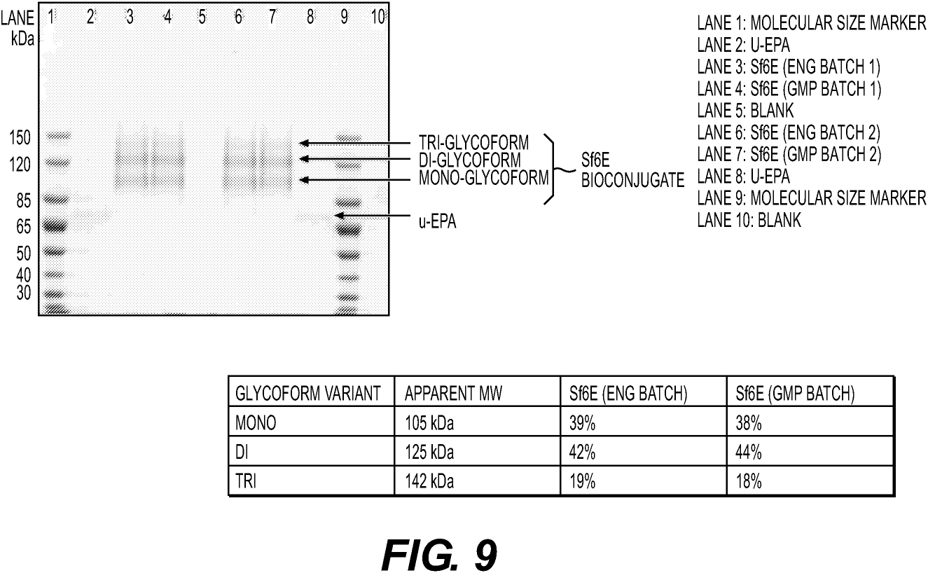

Sf6E ENG and GMP API batches showed a nearly identical degree of glycosylation with predominantly mono- and di-glycosylated forms, demonstrating the comparability of the two batches (FIG. 9).

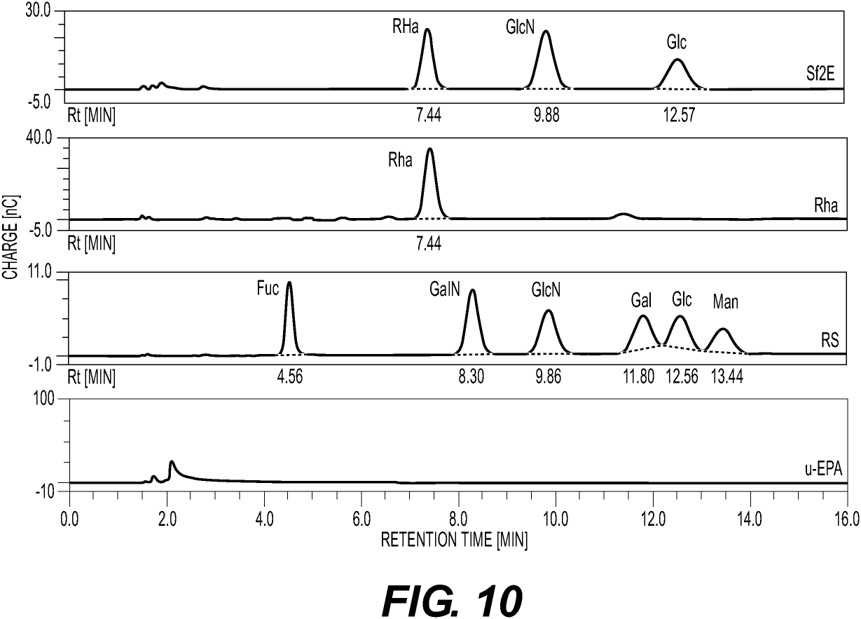

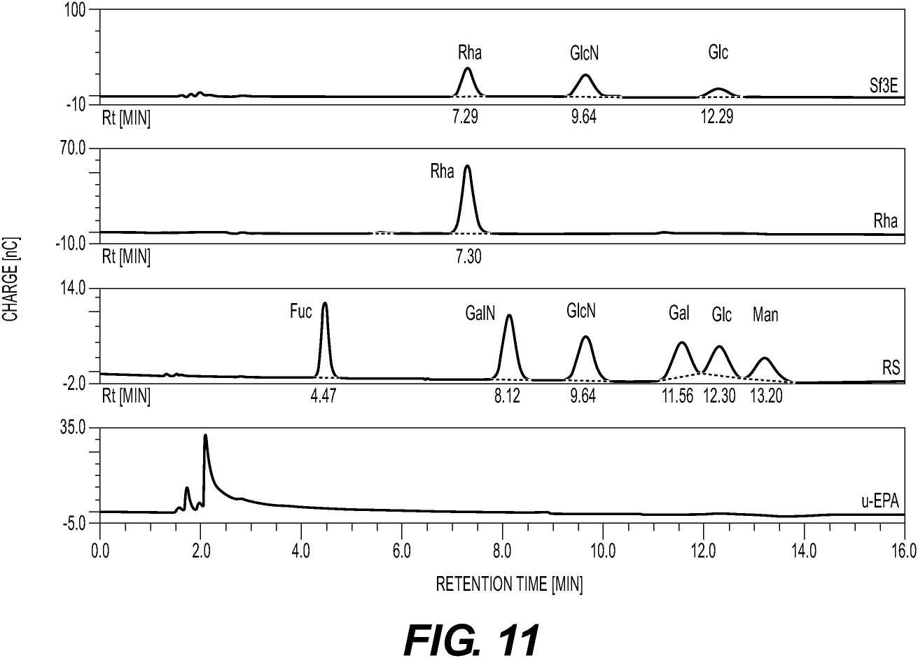

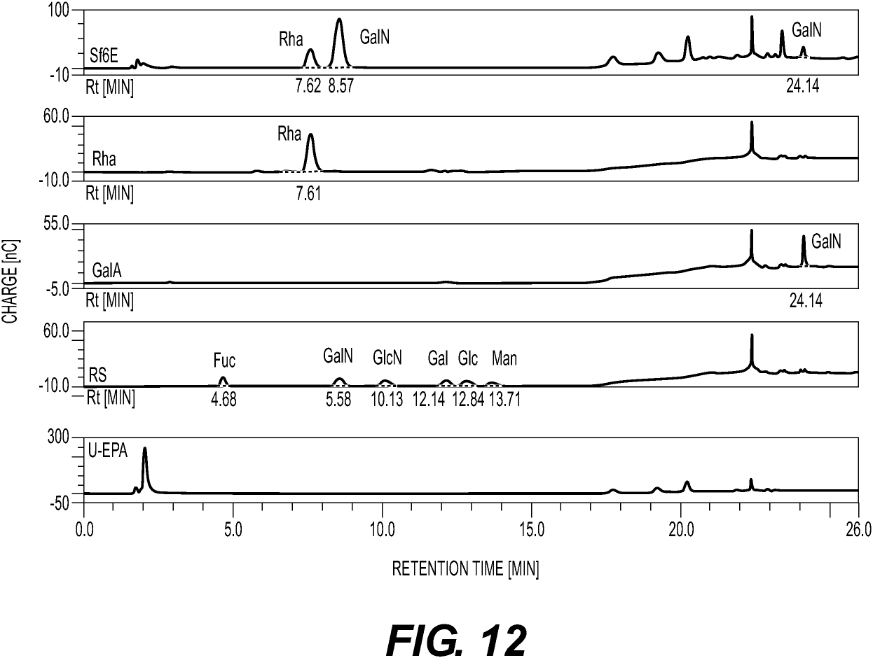

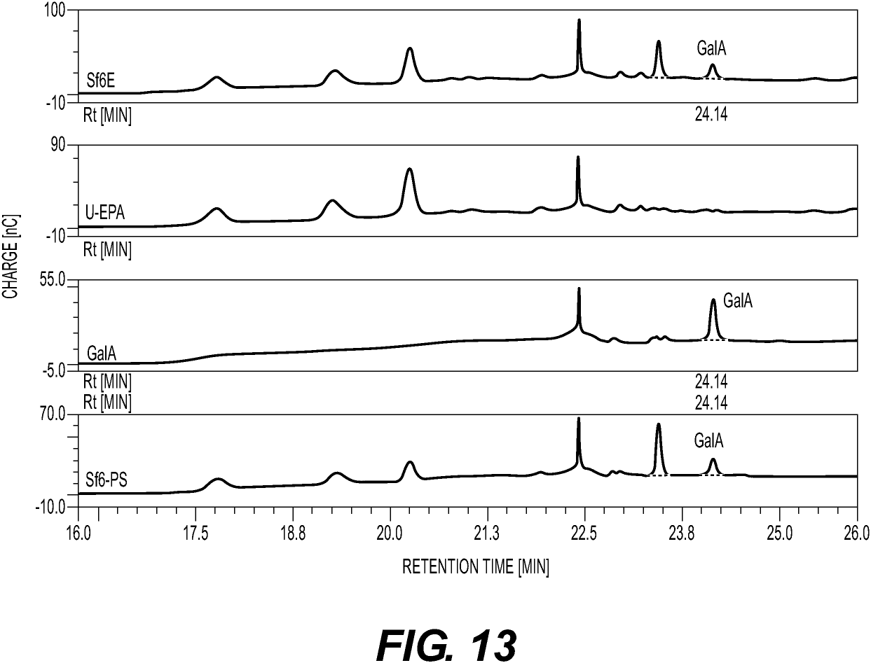

The monosaccharide composition of the three Shigella flexneri GMP API’s Sf2E, Sf3E and Sf6E was determined by high performance anion exchange chromatography coupled with pulsed amperometric detection (HPAEC-PAD) and the individual monosaccharides were identified by comparison to commercially available monosaccharide standards. The monosaccharides were released from the bioconjugate by TFA hydrolysis. The resulting underivatized monosaccharides are separated by column chromatography by eluting with NaOH/NaOAc and subsequent detection by pulsed amperometric detection (PAD). For Sf2E and Sf3E the monosaccharides Rha, GlcNAc (GlcN after TFA hydrolysis) and Glc could be verified by overlaying with the corresponding monosaccharide standards of the commercial reference solution (RS) and Rha monosaccharide standard (FIG. 10 and FIG. 11). The found monosaccharides confirm the monosaccharide composition of the Sf2a and Sf3a polysaccharide. For Sf6E the monosaccharides Rha, GalNAc (GalN after TFA hydrolysis) and GalA were confirmed by comparing to reference solution (RS) and GalA monosaccharide standard (FIG. 12). Beside the identified monosaccharide peaks, the Sf6E sample showed EPA related protein and Sf6 PS related glycan peaks (most probably amino acids, peptides and not completely hydrolyzed PS species eluting during the acetate gradient) which were assigned by analyzing in parallel with u-EPA and Sf6 PS samples (FIG. 13).

### Example 7: Glycan Structure Confirmation by Hydrazinolysis, Normal Phase (NP)- HPLC Followed by MALDI MS/ MS analysis

In order to determine the polysaccharide composition, sequence and length of the glycans conjugated to the carrier protein the ENG and GMP API batches were subjected to hydrazinolysis. This treatment allows for the chemically release of the polysaccharide chains from the carrier protein. Anhydrous hydrazine reacts at the linkage between the glycan and the backbone of the peptide and releases the glycan. The hydrazinolysis procedure involves several reaction steps including re-N-acetylation of the free amino groups and acid hydrolysis of the acid labile β-acetohydrazide derivative to produce the free glycans. The glycans are purified via ENVI Carb SPE columns, labeled with 2-AB at their reducing ends and separated by NP-HPLC. The resulting peak of interest were collected and subjected to MS/MS analysis by MALDI.

The re-acetylation is carried out because N-acetyl groups are lost during hydrazinolysis. Also, O-acetyl groups will be lost and therefore only structures without the O-acetyl groups are expected for the hydrazinolysis results, despite the O-acetyl groups expected for Sf3E and Sf6E bioconjugates.

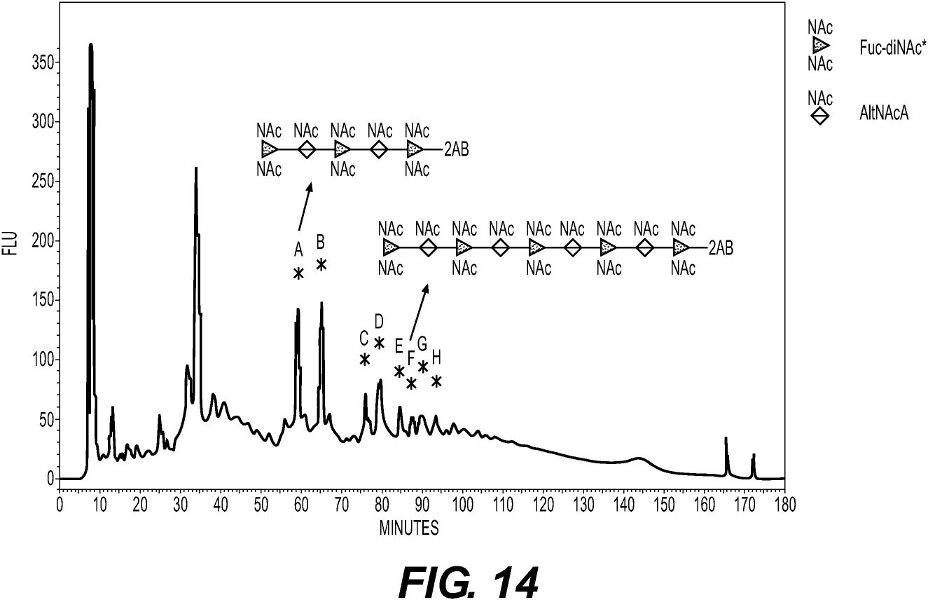

For the SsE ENG API batch (FIG. 14) two glycan species originating from the Ss glycan structures were confirmed for the peaks at a RT of 59.1 and 87.5 min. The structures confirmed correspond to 3 RU′s - AltNAcA (m/z 1257) and 5 RU′s - AltNAcA (m/z 2169) respectively. The FucNAc4N carries only one N-acetyl group on the amino group in position 2, which is lost during the hydrazinolysis. A reacetylation step during the hydrazinolysis sample processing re-N-acetylates the sugar not only in position 2 but also in position 4. The 2-acetamido-4-amino-2,4,6-trideoxy-D-galactopyranose (DFucNAc4N) becomes a 2, 4-diacetamido-2,4,6-trideoxy-D-galactopyranose, which corresponds to the confirmed masses in the MALDI measurements. Intact protein MS measurements confirmed by mass that FucNAc4N is present in the bioconjugate and that the average number of RU’s is around 29 for the SsE ENG API batch (data not shown). The SsE polysaccharide was strongly degraded by hydrazinolysis and no longer polysaccharide species were observed in the HPLC chromatogram. The confirmed species resemble fragments of longer polysaccharide chains. The fact that only forms with the modified FucNAc4N on the non-reducing end were found suggests that the α-1,3 linkage within the RU is weaker than the β-1,4 between the RU’s.

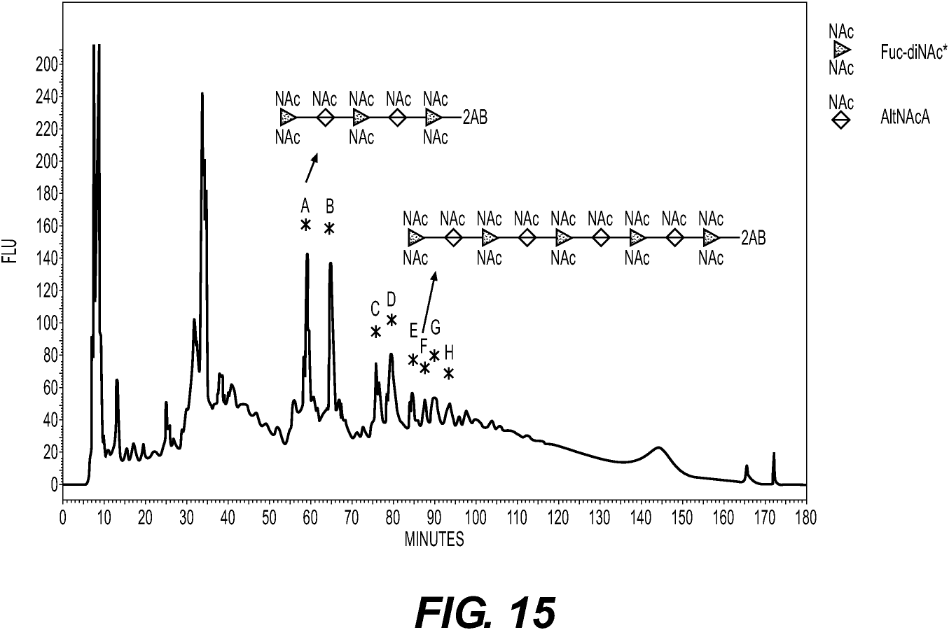

For the SsE GMP API batch (FIG. 15) the same two peaks at RT 59.1 and 87.6 min were collected and measured. The same structures as in the ENG API batch were confirmed, 3 RU′s - AltNAcA (m/z 1257, H-adduct) and 5 RU′s - AltNAcA (m/z 2169, Na-adduct) respectively. As for the SsE ENG batch 2, 4-diacetamido-2,4,6-trideoxy-D-galactopyranose was confirmed instead of 2-acetamido-4-amino-2,4,6-trideoxy-D-galactopyranose (D-FucNAc4N) because of re-acetylation during the hydrazinolysis and the re-acetylation step. Also, for the GMP API batch intact protein MS measurements confirmed the presence of FucNAc4N in the bioconjugate and the average number of RU’s determined was around 29 (data not shown). The HPLC trace of the GMP batch is comparable to the ENG batch, showing equal strong degradation of the polysaccharide.

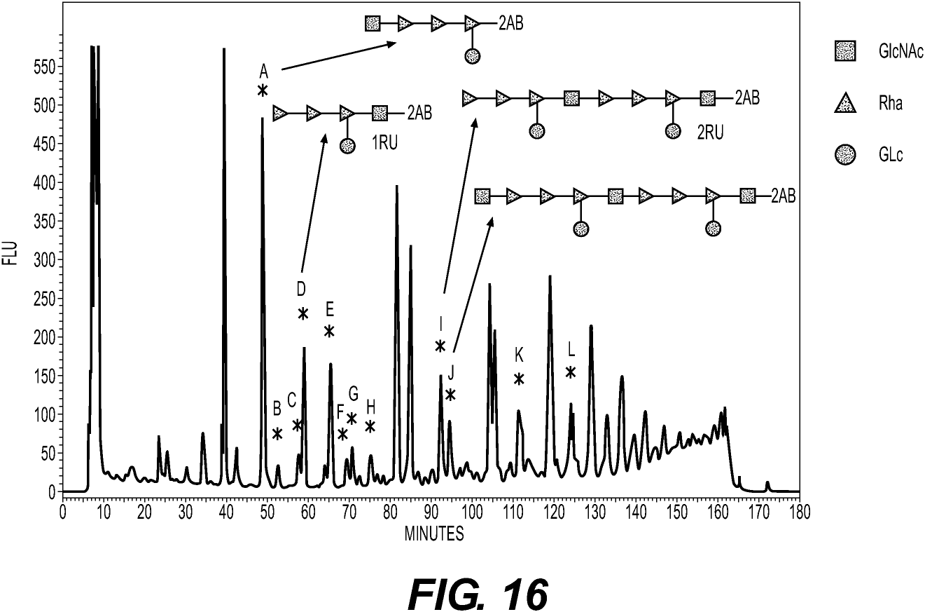

For the Sf2E ENG API batch (FIG. 16) the 1RU and 2RU were confirmed for the peaks at a RT of 59.0 (m/z 964, Na-adduct) and 92.4 min (m/z 1767, Na-adduct) respectively. Two additional fragments were confirmed for the peaks at RT 49.1 and 94.6, which correspond to HexNAc-dHexdHex-dHex(Hex)-2AB (m/z 964, Na-adduct) and HexNAc-2[dHex-dHex-dHex(Hex-)-HexNAc]-2AB (2RU+HexNAc Sf2a) (m/z 1970, Na-adduct) respectively. These two species resemble fragments of longer polysaccharide chains that are degraded during hydrazinolysis. Also, for the ENG batch several very lower abundant peaks were collected and subjected to MALDI measurements, however no structure of interest could be confirmed.

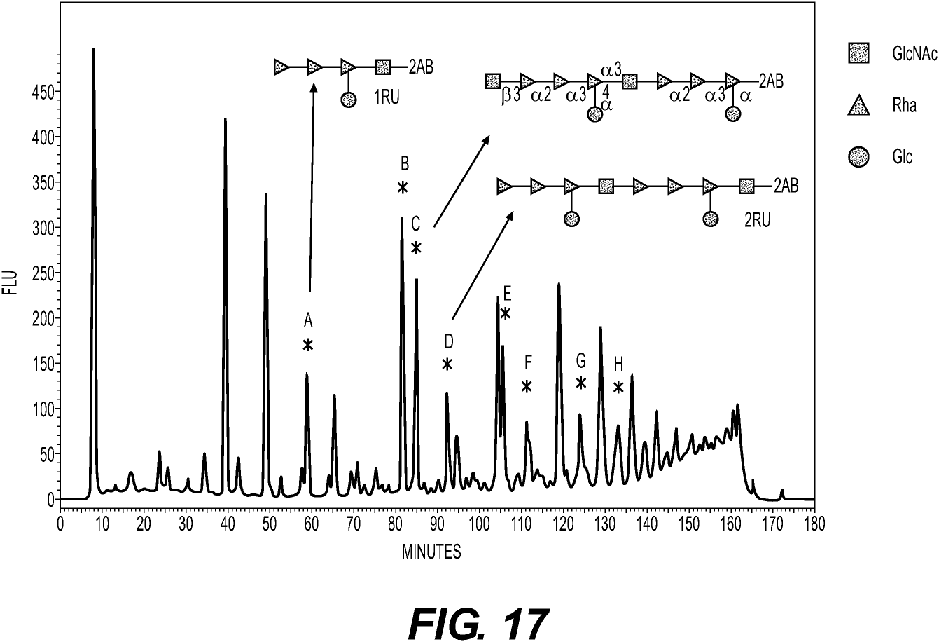

For the HPLC run of the Sf2E GMP API batch (FIG. 17) a comparable chromatogram to the ENG batch was obtained. Also 1 and 2 RU’s were confirmed for the peaks at RT 59.0 (m/z 964, Na-adduct) and 92.4 min (m/z 1767, Na-adduct) respectively. Further, the fragment > 2 RU’s, (HexNAc-dHexdHex-dHex(Hex-))2-2AB was confirmed for the peaks at RT 84.9 (m/z 1767, Na-adduct). Also, for the GMP batch several lower abundant peaks were collected and subjected to MALDI measurements, however the structures could not be entirely confirmed by MALDI-MSMS.

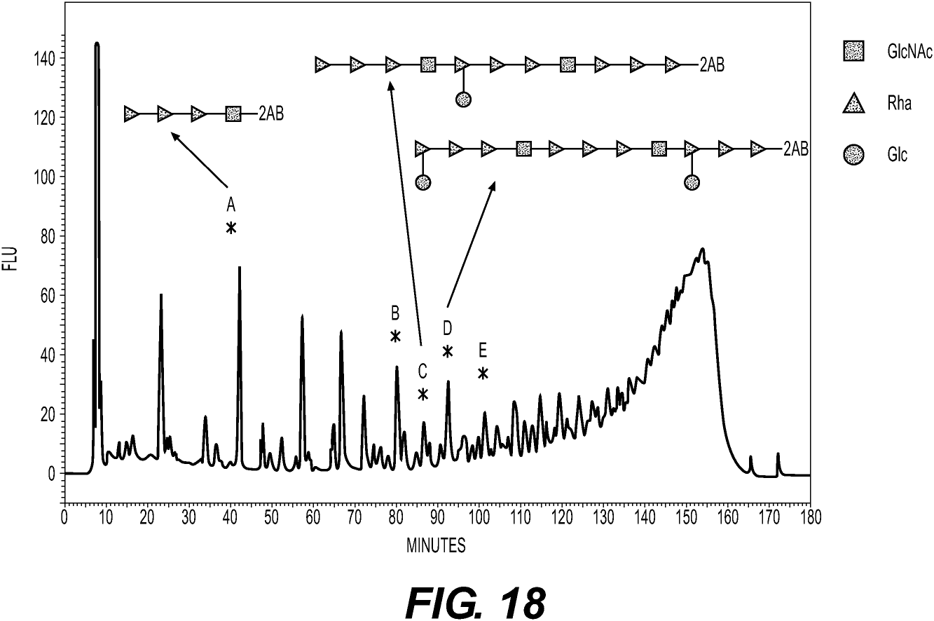

For the Sf3E ENG API batch (FIG. 18) three glycan species originating from the Sf3E polysaccharide structures were confirmed for the peaks at a RT of 42.4, 86.7 and 92.6 min. The structures confirmed correspond to fragments of 1 and 3 RU’s. At RT 42.4 dHex-dHex-dHex-HexNAc-2AB (m/z 780, H-adduct), at RT 86.7 dHex- dHex-dHex-HexNAc-dHex(Hex-)-dHex-dHex-HexNAc-dHexdHex-dHex-2AB (m/z 2043, Na-adduct) and at RT 92.6 dHex(Hex-)-dHex-dHex-HexNAc-dHexd-dHexdHex-HexNAc-dHex(Hex)-dHex-dHex-2AB (m/z 2206, Na-adduct) was confirmed. Two collected fractions could not be confirmed by MALDI-MS/MS. The fragments observed are assumed to be generated during hydrazinolysis, which is a very harsh reaction, leading to the partial degradation of the Sf3E polysaccharide structure.

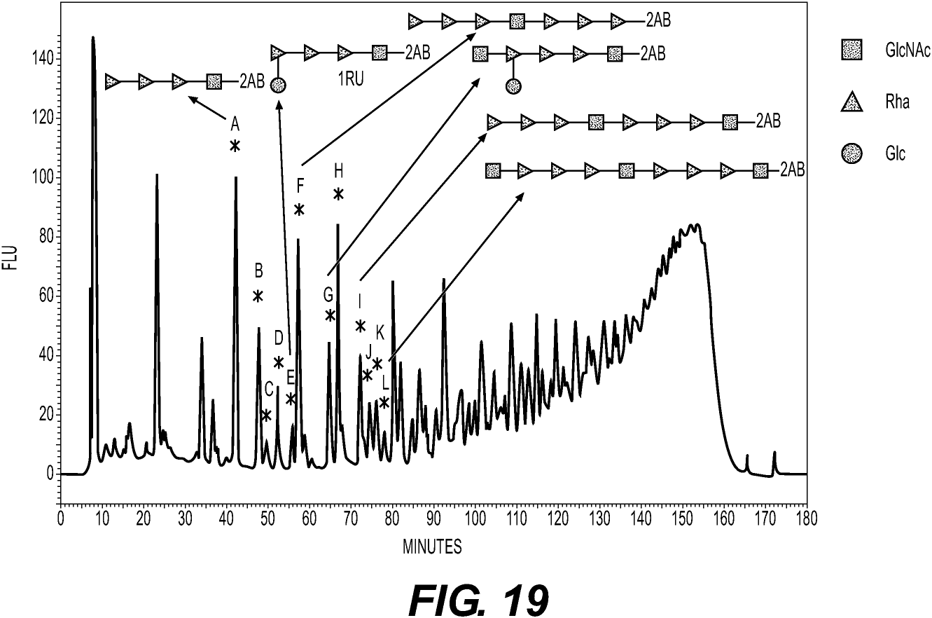

For the Sf3E GMP API batch (FIG. 19) the chromatogram looked comparable to the ENG batch and several glycan species originating from the Sf3E polysaccharide were confirmed by MALDI-MS/MS, including the 1RU at RT 56.0 (m/z 964, Na-adduct). All the other structures confirmed were fragments of the Sf3E polysaccharide, dHex-dHex-dHex-HexNAc-2AB was confirmed at RT 42.6 (m/z 780, H-adduct), dHex-dHex-dHex-HexNAc-dHex-dHex-dHex-2AB at RT 57.6 (m/z 1240, Na-adduct), HexNAc-(Hex-)-dHex-dHex-dHex-HexNAc-2AB (1RU+HexNAc Sf2a) (m/z 1167, Na-adduct), dHexdHex-dHex-HexNAc-dHex-dHexdHex-HexNAc-2AB at RT 72.4 (m/z 1443, Na-adduct) and dHex-dHexdHex-HexNAc-dHex-dHexdHex-HexNAc-2AB at RT 78.1 (m/z 1646, Na-adduct).

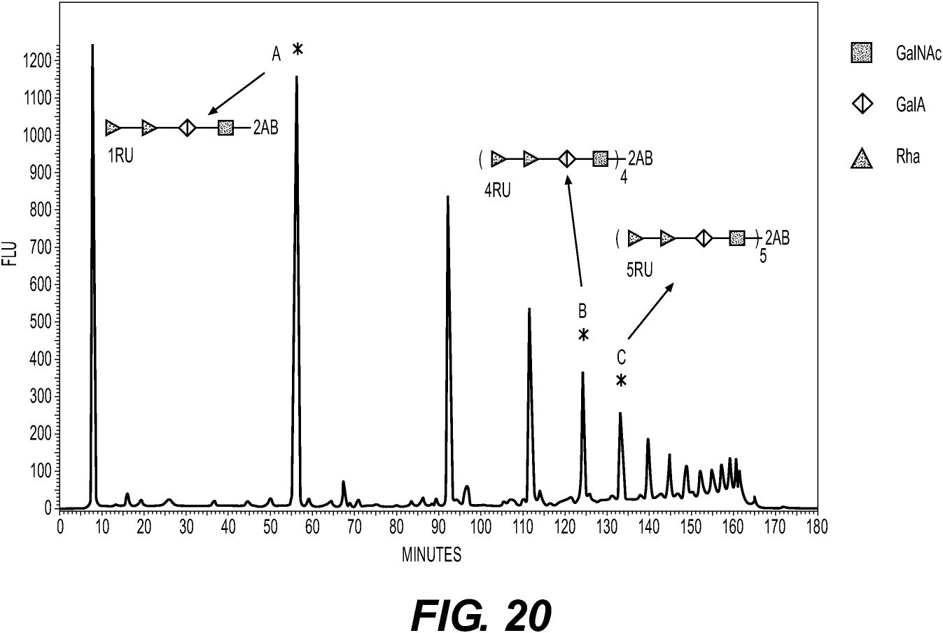

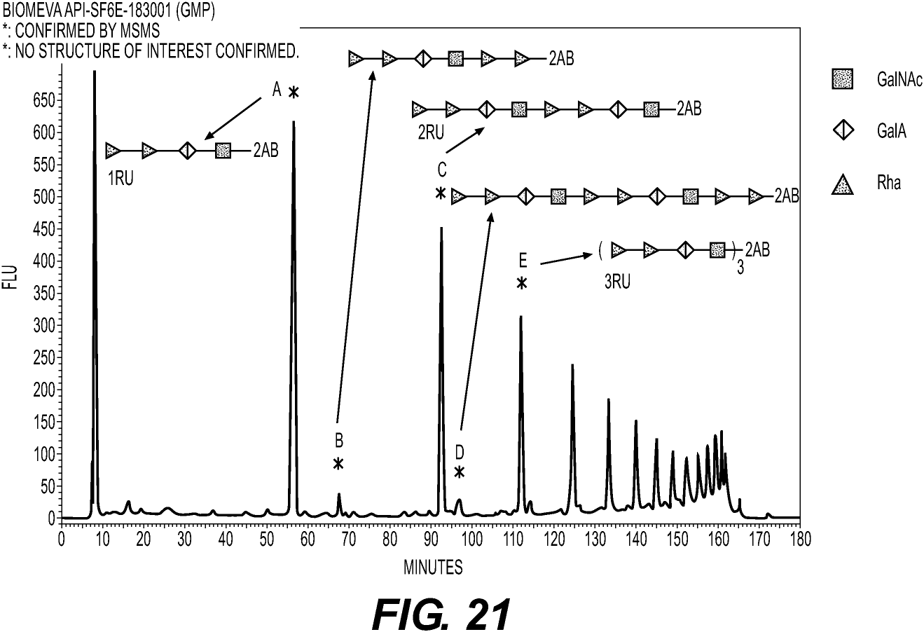

For the Sf6E ENG API batch (FIG. 20) 1 RU, 4 RU and 5 RU glycan species were confirmed for the peaks at a RT of 56.5 (m/z 810, H-adduct), 124.5 (m/z 2846, Na-adduct) and 133.3 (m/z 3517, Na-adduct) min.

For the Sf6E GMP API batch (FIG. 21) 1 RU, 2 RU and 3 RU glycan species were confirmed for the peaks at a RT of 56.5 (m/z 810, H-adduct), 92.6 (m/z 1481, H-adduct) and 111.9 (m/z 2174, H-adduct) min. Further, for two minor peaks at RT 67.7 and RT 96.9 the fragments dHex-dHex-HeAHexNAc-dHex-dHex-2AB (m/z 1124, Na-adduct) and dHex-dHex-HexA-HexNAc-dHex-dHex-HexAHexNAc-dHex-dHex 2AB (m/z 1795, Na-adduct) were confirmed. It should be mentioned that the chromatograms of the HPLC runs in ENG and GMP batch look very similar.

### Example 8: S. Sonnei NMR Data

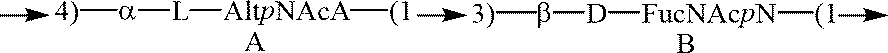

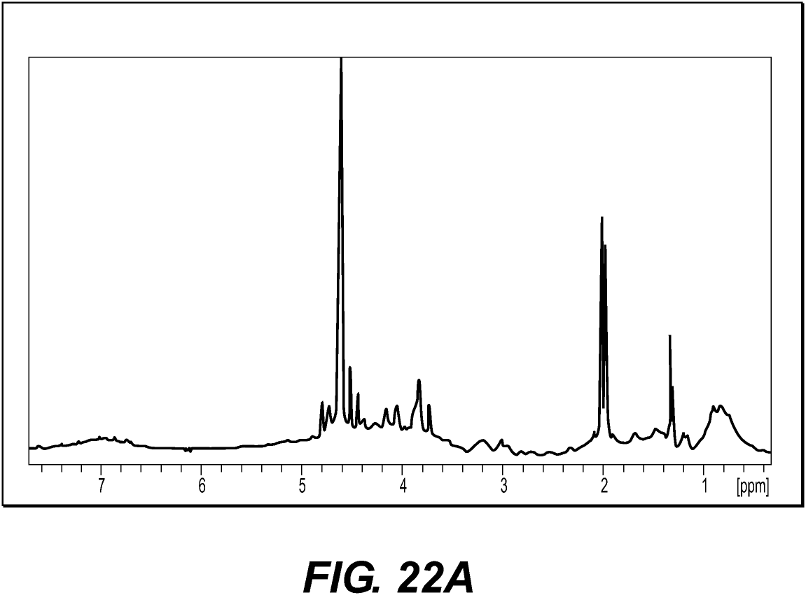

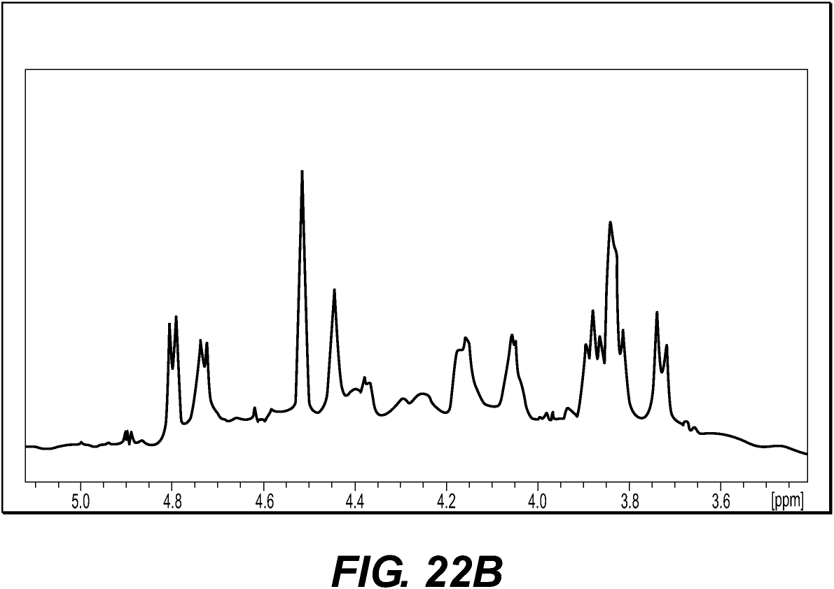

Spectra were referenced relative to H6/C6 of β-FucNAc4N: 1H at 1.33 ppm, 13C at 16.31 ppm. The 1H NMR spectrum of the SsE bioconjugate (FIG. 22A) contains sharp signals due to the S. sonnei disaccharide RU superimposed on broad peaks of low intensity from the EPA protein. The saccharide peaks are characteristic of the S. sonnei RU: one α- and one β-linked sugar, ring protons, two N-acetyl and a methyl group (from β-FucNAc4N). The 1D DOSY expansion (FIG. 22B), which removes the large HOD signal, allows assignment of all the signals for the disaccharide S. sonnei RU in the anomeric and ring regions.

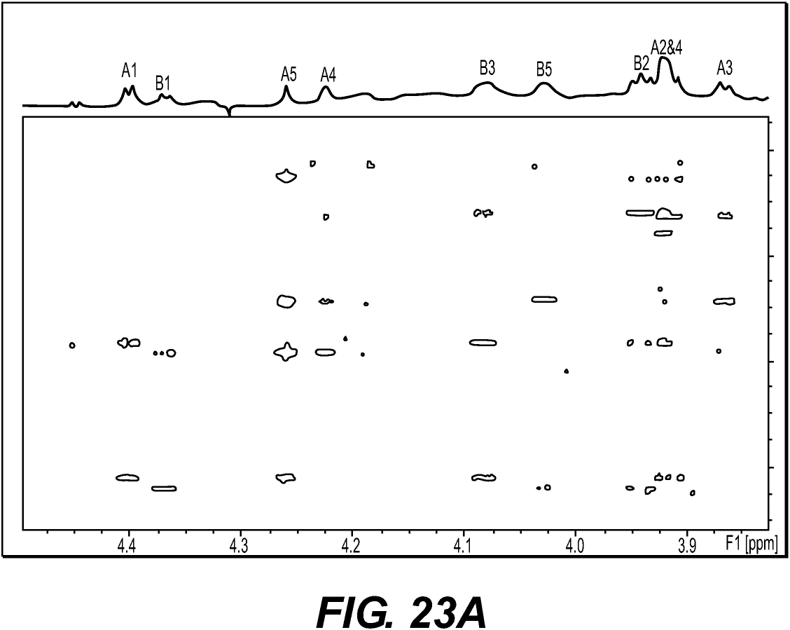

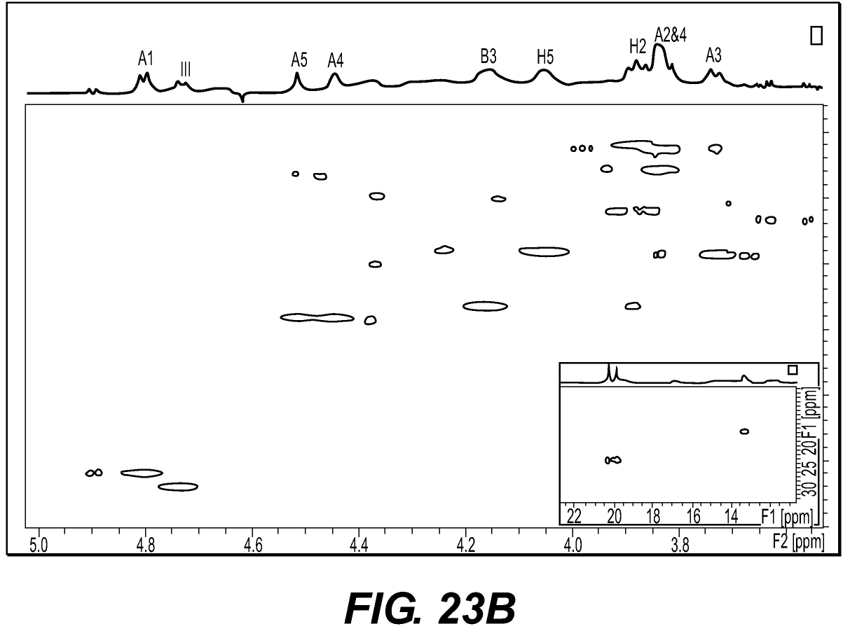

Corroboration of the assignments of the two spin systems was aided by the use of the HMBC experiment. An overlay of HSQC /HMBC (black) in FIG. 23 (left panel) shows 2- and 3-bond correlations for the disaccharide RU and key inter-residue correlations which confirm the linkages and sequence of residues in the disaccharide RU of S. sonnei. The methyl region (data not shown) gave correlations from H6 of FucNAcN to C5 and C4 of the same residue, whereas the methyls of the N-acetyl groups gave crosspeaks to the corresponding C=O groups. Lastly, H5 of AltNAcA gave a crosspeak to C6, the carboxyl carbon at 172.8 ppm.

NMR analysis confirms the structure of the biosynthetically produced disaccharide RU of S. sonnei as →4)-α-AltpNAcA-(1→3)-β-FucpNAcN-(1→. The 1D proton and 2D 1H-1H (TOCSY) and 1H-13C (HSQC) spectra assigned in this study constitute identity maps of the S. sonnei antigen.

### Example 9: The Manufacturing Process

The manufacturing processes for all four drug substances Sf2E, Sf3E, Sf6E and SsE initiate with a single vial of the corresponding master cell bank.

The upstream processing (USP) process steps embodies inoculation, Fed batch fermentation, harvest and wash by centrifugation, and storage.

The downstream processing (DSP) process steps are similar for Sf2E, Sf3E Sf6E, and SsE. The steps include, osmotic shock, centrifugation, column chromatography, tangential flow filtration, size exclusion chromatography or ion exchange chromatography, and storage.

### Example 10: Description and Composition of the Immunogenic Composition

The tetravalent bioconjugate vaccine candidate to be evaluated in the present clinical trial is intended for intramuscular administration and consists of the four Immunogenic Composition components Sf2E, Sf3E, Sf6E and SsE in an 1:1:1:1 ratio formulated as a liquid dosage form in 10 mM sodium phosphate pH 6.5, 150 mM sodium chloride 0.015% Polysorbate 80 (PBS pH 6.5 + 0.015% Polysorbate 80) and stored at 2 - 8° C. Between 1 and 48 µg glycan per serotype after on-site dilution with Diluent or Adjuvant. The adjuvant may be Aluminium hydroxide (1.6 mg Aluminum/mL)) diluted in 150 mM NaCl in water for injection (WFI). The diluent may be 10 mM sodium phosphate, pH 6.5, 150 mM NaCl.

### Example 11: Formulation Development

The formulation pH has been chosen since O-acetyl groups are labile at a basic pH. Furthermore, the carrier protein EPA is not stable below a pH of 5.5. Consequently, the pH was set to 6.5. 150 mM sodium chloride is used to achieve isotonicity.

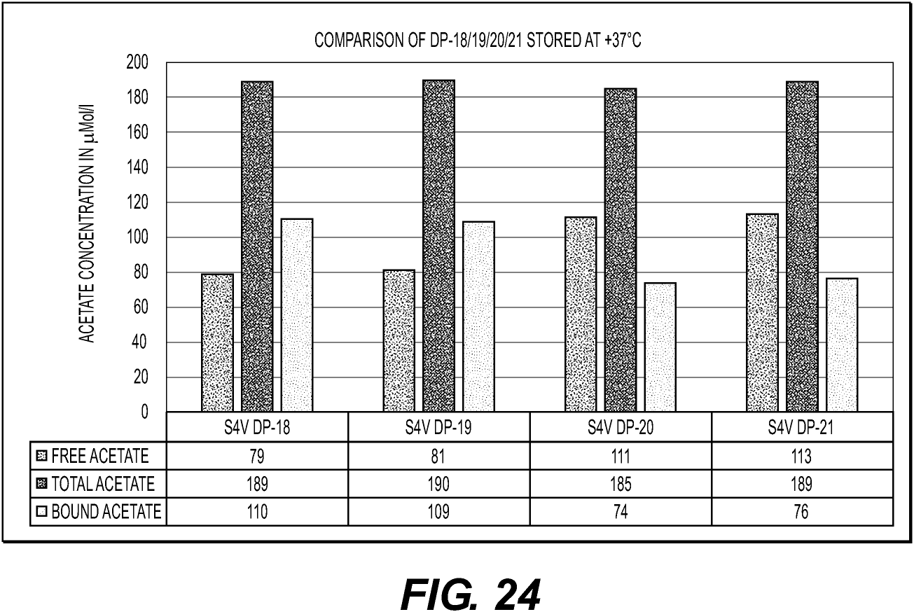

Formulation experiments were conducted, including different relevant stress conditions, such as temperature, freeze-thaw, shear force, agitation stress as well as container closure adsorption. The different studies revealed that the formulation 10 mM sodium phosphate, pH 6.5, 150 mM NaCl sufficiently stabilizes the Immunogenic Composition with regards to temperature stress, adsorption and O-acetyl stability. Freeze-thaw and agitation stress however resulted in an increase in particle size, which was prevented when adding Polysorbate 80. This behavior was confirmed with complimentary analytical techniques in different experiments. Additional formulations tested so far did not result in a superior stabilization as compared to Polysorbate 80. Hence it was decided to further stabilize the formulation by the addition of 0.015% Polysorbate 80. Polysorbate 80 is a widely used excipient in vaccine formulations in similar concentrations (e.g. Prevnar 13 contains 0.02% w/w). The stability of the O-acetyl groups is reduced at higher pH in a time and temperature dependent manner, whereby pH 6.5 is preferred over pH 7.0 (FIG. 24).

### Example 12: Animal Study

The study was conducted in female New Zealand white rabbits. Animals were administered i.m. with either 1 µg PS dose of each vaccine component with and without Al(OH)₃ or 1 µg PS dose of a single vaccine component. One control group received injections with formulation buffer only and the other control group received no injections at all. The test items were administered on day 0, 14 and 28 and serum was collected from all animals prior to the first injection and 14 days after the second (day 28) and third injection (day 42).

### Example 13: Anti-LPS IgG ELISA

Serum IgG titers specific for Sf2a-LPS, Sf3a-LPS, Sf6-LPS and Ss-LPS were measured by ELISA.

Microtiter 96-well plates (MAXISORPTM, Nunc, Thermo Scientific) were coated with 100 µI per well of 5 µg/ml LPS and 10 µg/ml of methylated BSA in PBS. After incubation over night at 4° C., the plates were washed with PBS 0.05% Tween®20. After washing, all wells were incubated for 2 hours with 300 µl of PBS 5% skimmed milk powder. After washing, plates were stored at -24° C. until further use. Plates were removed from the freezer and washed with PBS 0.05% Tween®20. Then serial three-fold dilutions (in PBS Tween®20 0.05%) of test sera were added in duplicates. The plates were incubated for 1 hour at room temperature under shaking. After washing, peroxidase- conjugated IgG-specific antibodies were added for 1 hour at room temperature under shaking goat anti-rabbit IgG(Fc) antibodies. Plates were washed as above and TMB substrate solution (Sigma T4444) was added to each well (100 µl/well) for 6 min. The reaction was stopped by addition of 100 µl of H₂SO₄ 1 N and the optical density (OD) was read at 450 nm. The individual endpoint titers were determined as the highest dilution above the mean OD value + 3 S.D. of the buffer only controls.

### Example 14: Anti-EPA IgG ELISA

EPA-specific serum IgG titers were measured by ELISA.

Microtiter 96-well plates were coated with 100 µl per well of 2 µg/ml uEPA (batch: E-7) in PBS. After incubation over night at 4° C., the plates were washed with PBS 0.05% Tween®20. After washing, all wells were incubated for 2 hours with 300 µl of PBS 5% skimmed milk powder. After washing, plates were stored at -24° C. until further use. Plates were removed from the freezer and washed with PBS 0.05% Tween®20. Then serial three-fold dilutions (in PBS Tween®20 0.05%) of test sera were added in duplicates. The plates were incubated for 1 hour at room temperature under shaking. After washing, peroxidase-conjugated IgG-specific antibodies were added for 1 hour at room temperature under shaking goat anti-rabbit IgG (Fc) antibodies. Plates were washed as above and TMB substrate solution was added to each well (100 µl/well) for 6 min. The reaction was stopped by addition of 100 µl of H₂SO₄ 1N and the optical density (OD) was read at 450 nm. The individual endpoint titers were determined as the highest dilution above the mean OD value + 3 S.D. of the buffer only controls.

### Example 15: SBA

The ability of serum antibodies to mediate killing (SBA) of the different Shigella serotypes in the presence of complement was assessed in a microtiter assay. An SBA titer is reported as the dilution of serum giving 50% killing of a specified inoculum of bacteria after incubation with test serum in the presence of an endogenous complement source.

Serum bactericidal activity was determined. For this assay pools of pre-immune and post-III immune sera of rabbits immunized with Shigella4V and buffer only were tested in SBA with S. flexneri 2a, S. flexneri 3a, S. flexneri 6, and S. sonnei.

To perform the assay the complement in immune sera was inactivated by incubation at 56° C. for 30 minutes. Duplicates of the complement-inactivated immune sera were three-fold serially diluted on a microtiter plate. To control for inactivation of complement the lowest serum dilution duplicates were prepared for incubation with (complement control, CC) and without (viable cell count, VCC) rabbit complement. Heat-inactivated rabbit complement was added to the VCC and complement-independent controls (CIC). S. flexneri 2a, S. flexneri 3a, S. flexneri 6 and S. sonnei were grown overnight at 37° C. with 5% CO₂ on a Tryptone soya agar (TSA) plate and were harvested and suspended in buffer and adjusted to a concentration of 0.1 OD₆₀₀. This suspension was diluted further 1:5000 and 10 µl of this suspension was added to the diluted sera and incubated at 37° C. for 60 minutes with shaking (iEMS Microplate Incubator/Shaker). Rabbit complement was added at 25% of the volume in the microtiter wells and incubated at 37° C. for a further 60-75 minutes with shaking. At the end of the incubation 10µl from each well were dotted onto pre-labelled TSA. Each sample was plated in duplicate on TSA plates, and incubated overnight at 26° C. with 5% CO₂ (16 - 18 hours). The % survival of bacteria was calculated using the formula CFU from test sera/CFU from viable cell count control (VCC) control x 100).

The SBA titers were calculated by determining the mean of the active complement control wells in each assay and dividing the mean absolute titer by 2; establishing a 50% cutoff value. The titer was determined to be the reciprocal of last sample dilution that has a colony count of ≤ the 50% cutoff value. The interpolated titer was determined using the 50% cutoff value and was calculated by curve fitting. The Opsotiter software uses colony counts from two sequential dilutions of serum, one that kills less than 50% and one that kills more than 50%, by applying the algorithm:

$$\text{OI} \left(\text{X50}\right) = {10}^{\left(log \text{X1+} \frac{\left(\text{Y50} - \text{Y1}\right) \times \left(log \text{X2} - log \text{X1}\right)}{\left(\text{Y2} - \text{Y1}\right)}\right)}$$

### Example 16: LPS-Specific IgG Responses

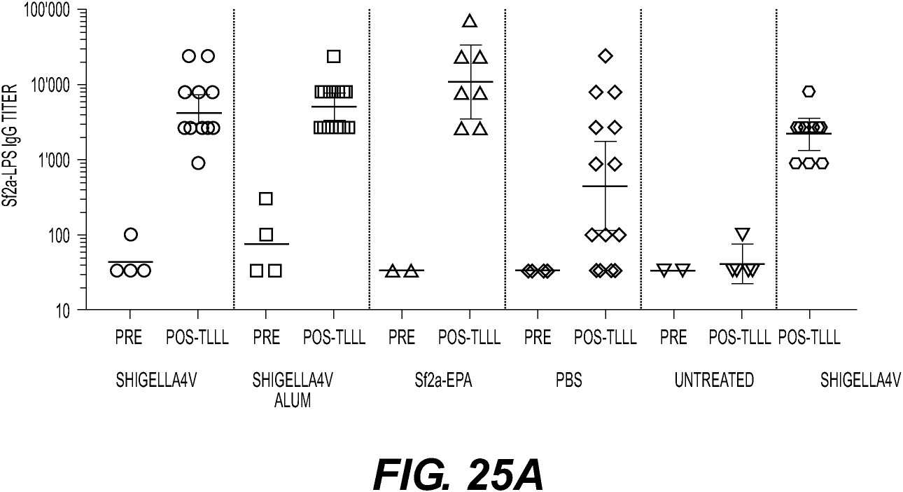

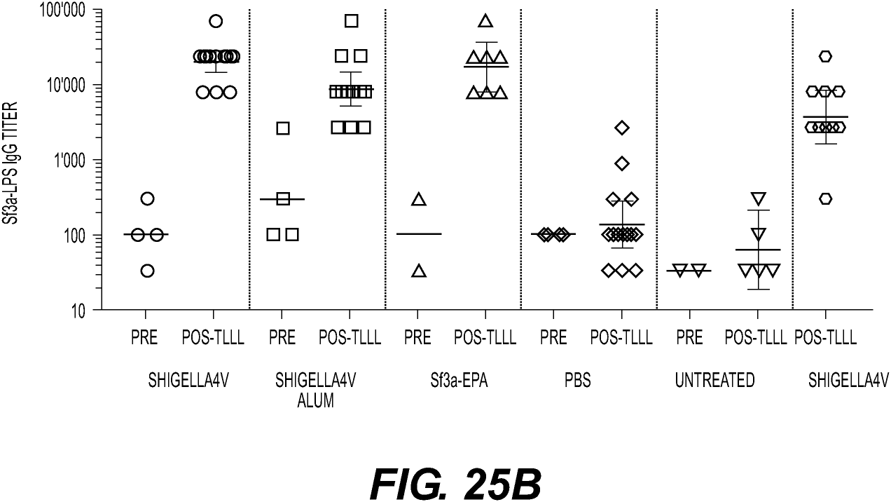

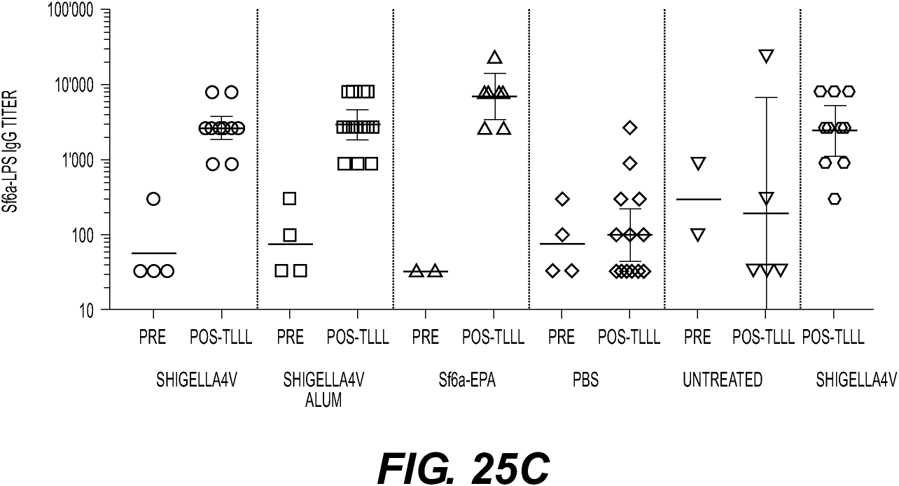

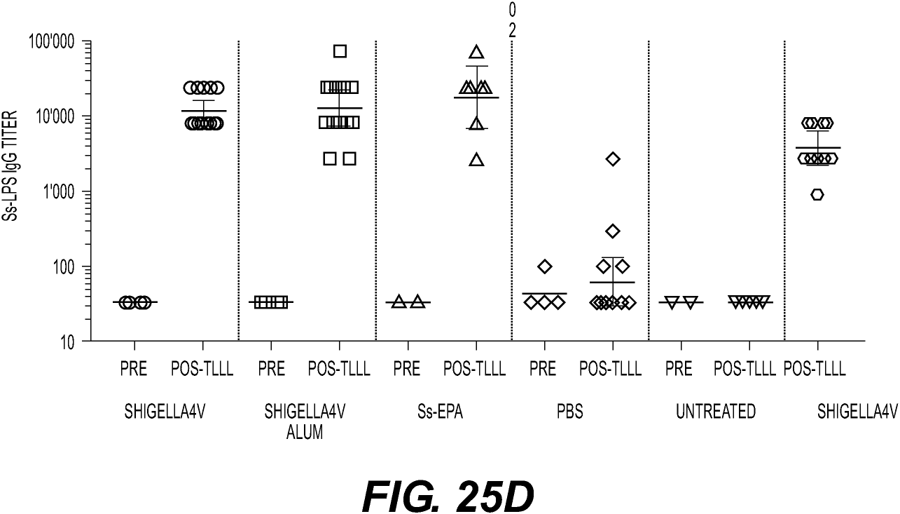

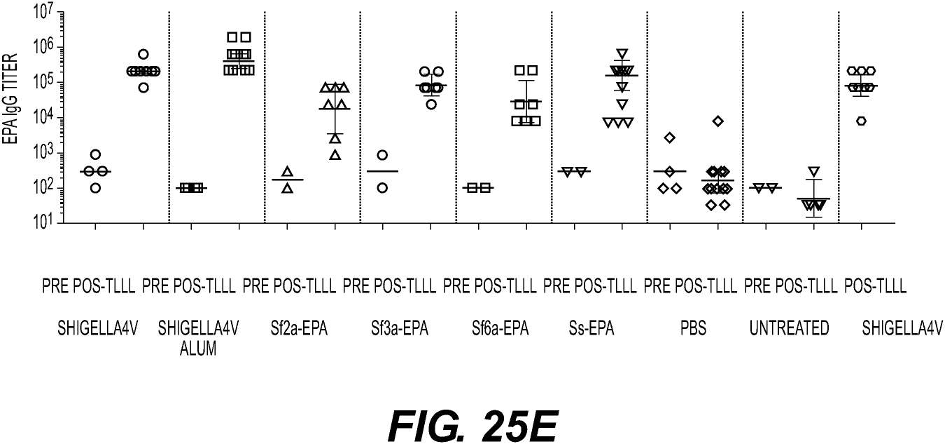

GMTs over time are shown in FIG. 25A FIG. 25B, FIG. 25C FIG. 25D, and FIG. 25E.

In a fraction of pre-immune serum pools Sf2a-, Sf3a- and Sf6-LPS-specific IgG titers were detectable, indicating that some animals already have serum IgG that bind to these LPSs. Similarly, a fraction of post-III immune sera of rabbits treated with PBS buffer only, as well as untreated rabbits, contained IgG that bind to these LPSs.

The post-III IgG titers of PBS treated animals and untreated animals were not statistically significant different (p≥0.7932), except for Sf2a-LPS specific IgG titers, that were 10.7-fold higher in the PBS treated animals (p=0.0136).

Vaccination of rabbits with Shigella4V elicited IgG responses against all 4 O-antigen components. Post-III titers against all 4 LPSs were significantly higher in rabbits vaccinated with Shigella4V compared to rabbits injected with PBS only (p≤0.0004). GMR between the Shigella4V and the PBS group were 9.7 for Sf2a-LPS, 151.7 for Sf3a-LPS, 27.0 for Sf6-LPS and 191.9 for Ss-LPS titers.

The Shigella4V vaccine was also immunogenic when formulated with Al(OH)₃. The post-III serum titers were significantly higher compared to the PBS treated group (p≤0.001). The GMR between the Shigella4V Alum group and the PBS group was 11.4 for Sf2a-LPS, 64.0 for Sf3a-LPS, 29.2 for Sf6-LPS and 207.5 for Ss-LPS.

The formulation of Shigella4V with Al(OH)₃ had no significant effect on the LPS-specific vaccine responses (p≥0.2108). GMRs between the Shigella4V and Shigella4V Alum group were 0.9 for Sf2a-LPS, 2.4 for Sf3a-LPS, 0.9 for Sf6-LPS and 0.9 for Ss-LPS.

Also, each of the monovalent vaccines elicited strong vaccine-specific anti-LPS IgG responses. The post-III serum titers were significantly higher than in the PBS treated group(p≤0.0001). The GMR between the monovalent treatment groups and the PBS group was 24.9 for Sf2a-EPA, 129.7 for Sf3a-EPA, 69.2 for Sf6-EPA and 284.4 for Ss- EPA.

The LPS-specific IgG responses in the 4-valent group was not significantly different to the response levels measured in the monovalent groups (p≥0.7735), indicating no major interference effect of the multivalent formulation. The GMR between LPS-specific post-III IgG titers of the Shigella4V-immunized group and the group immunized with the monovalent vaccines was 0.4 for Sf2a-EPA, 1.2 for Sf3a-EPA, 0.4 for Sf6-EPA and 0.7for Ss- EPA.

### Example 17: Serum Bactericidal Activity of Antibodies Induced by Vaccination with Shigella4V

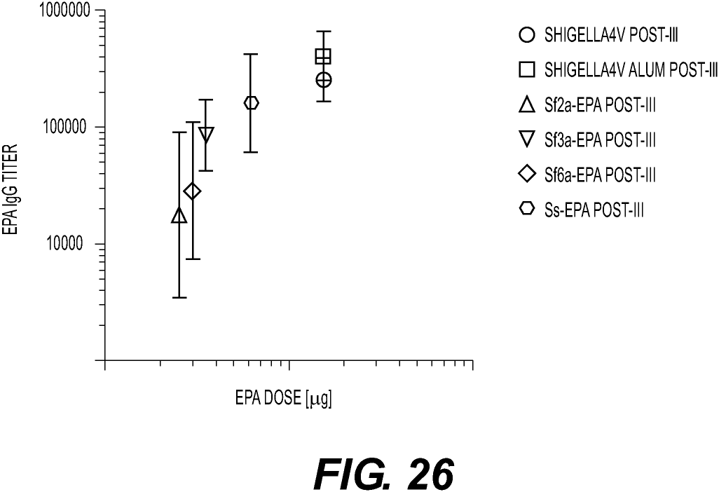

The results of SBA titers in the pre- and post-III sera in rabbit with monovalent vaccines and Shigella4V formulated with and without adjuvant are described in FIG. 26.

### Control Groups

Samples from the null treatment control groups had similar SBA titers across all shigella serotypes with the exception of S. flexneri 6 where 3-fold increase in SBA activity was noted in post versus pre-pre versus post-immunization pools. Rabbit control samples (PBS-immunized groups) also generally had low SBA titers in pre and post immunization time points across all Shigella serotypes, with the exception of S. flexneri 2a.

### Experimental Groups

All groups immunized with either monovalent or tetravalent vaccine showed good SBA titers/responses on post-III. Similar SBA titers were achieved after vaccination with the tetravalent vaccine as compared to the tetravalent vaccine co-administered with adjuvant, suggesting the adjuvant did not positively or negatively influence the generation of bactericidal antibodies. SBA titers achieved after immunization with monovalent formulations were comparable (within 2-fold) to SBA titers achieved after immunization with tetravalent vaccine formulation, suggesting minimal immune interference in terms of bactericidal activity. S. sonnei monovalent presented 4-fold higher titer than the tetravalent vaccine. The anti-S.flexneri 2a SBA results indicate that the monovalent Sf3a vaccine induces some cross reactivity with Sf2a, albeit at lower levels that what was achieved after immunization with Sf2a bioconjugate. Interestingly, serum from rabbits immunized with Sf2a did not possess comparable levels of Sf3a-specific bactericidal activity.

### Example 18: Evaluation of Mutated Forms of PalB Oligosaccharyltransferase for Their Ability to Glycosylate an Asparagine Residue with the Saccharide of Shigella Sonnei

Variants of PgIB were tested for their ability to catalyse the glycosylation of Exoprotein A from P. aeruginosa (EPA) containing D/E-Z₁-N-Z₂-S/T glycosylation sites (where Z₁ and Z₂ are not P) using a polysaccharide corresponding to that of Shigella sonnei O-antigen. Therefore a E. coli host cell was transformed with plasmids encoding glycosyltransferase genes required for the construction of a S. sonnei O-antigen, a variant PgIB gene and EPA containing glycosylation sites. Expression of the genes was induced using IPTG and arabinose and the E. coli host cells were grown overnight to allow expression of glycosyltransferases, PgIB and EPA and glycosylation of EPA as follows.

The wells of a 96 deep well plate were filled with 1 ml of TB media and each well was inoculated with a single colony of host cell E. coli and incubated at 37° C. overnight. Samples of each well were used to inoculate main cultures in a 96 deep well plate containing of 1 ml of TB supplemented with 10 mM MgCl₂ and appropriate antibiotics and were grown until an OD600 of 1.3-1.5 was reached. Cells were incubated with 1 mM IPTG and 0.1% arabinose overnight at 37° C.

Periplasmic extracts were made by centrifuging the plates, removing supernatant and adding 0.2 ml of 50 mM Tris-HCl pH 7.5, 175 mM NaCl, 5 mM EDTA followed by shaking at 4° C. to suspend the cells. 10 µl of 10 mg/ml polymyxin B was added to each well and the cells were incubated for 1 hour at 4° C. The plate was centrifuged and the supernatant removed.

In order to isolate the glycosylated protein from the periplasmic extract, 120 µl of a 25% slurry of IMAC resin in 30 mM Tris pH 8.0, 10 mM imidazole, 500 mM NaCl was added to each well of a 96 well filter plate (Acroprep Advance) and placed on top of a Nunc ELISA plate. The plate was centrifuged and the flow through discarded. 150 µl of periplasmic extract and 37.5 ml of 5x binding buffer (150 mM Tris pH 8.0, 50 mM imidazole, 2.5 M NaCl was added to each well. The samples were incubated for 30 minutes at room temperature. The plate was centrifuged and the flow through discarded and three more washing steps were carried out. Finally, the glycosylated protein was eluted with 30 mM Tris pH 8.0, 500 mM imidazole, 200 mM NaCl, ready for use in an ELISA assay.

A sandwich ELISA was performed by coating the wells of a 96-well plate with an antibody that recognizes the saccharide part of the glycosylated protein (for example, a monoclonal antibody against S. aureus capsular polysaccharide type 5) diluted in PBS. The plate was incubated overnight at 4° C. to allow coating. The plate was then washed with PBS containing 0.1% Tween. The plate was then blocked for 2 hours at room temperature using 5% bovine serum albumin in PBST. The plate was washed in PBST. The sample was diluted in PBST containing 1% BSA and incubated in the coated wells for one hour at room temperature. After washing a detection antibody, for example anti-Histag - horseradish peroxidase diluted in PBST containing 1% BSA was added to each well and incubated for one hour at room temperature. The plate was then washed before adding 3,3’,5,5′-Tetramethylbenzidine liquid substrate, Supersensitive, for ELISA (Sigma-Aldrich). After a few minutes, the reaction was stopped by addition of 2 M sulfuric acid. The results were obtained by reading the OD at 450 nm.

### Results

As a starting point, mutations were generated in a PgIB which already contained mutations at N311V and Y77H. A PgIB containing mutations at N311V and Y77H was subjected to mutation and promising variants were selected, sequenced and analysed for OST activity as described above. The fold increase in oligosaccharyltransferase activity of each variant was calculated and the results are shown in Table 5.

TABLE 5

Improvement in engineered PgIB OST activity in transferring S. sonnei O-antigen to a protein as determined by ELISA

| | | |
|---|---|---|
| PgIB Variant mutation | Amino acid substitution | Fold increase in OST activity |
| G55 | M | M-1.972 |
| Y78R | R | R-1.503 |
| I101 | C | C-2.508 |
| R125 | T | T-3.296 |
| T153 | P | P-1.857 |
| Y155 | H | H-1.664 |
| Y191 | T | T-1.473 |
| W192 | R | R-1.437 |
| 1273 | R | R-1.436 |
| D282 | L, P | L-2.184 |
| L300 | P | P-1.612 |
| Q315 | Y | Y-3.516 |
| L371 | H | H-2.36 |
| Y425 | T, P | T-1.863, P-1.799 |
| Q435 | L | L-1.491 |
| Y466 | T | T-1.466 |
| V474 | A | A-1.52 |
| G477 | A | A-1.759 |
| F513 | Y | Y-1.542 |
| R570 | G, A, D | G-1.467, A-1.439 |
| l581 | G, A | G-1.459 |
| S610 | W | W-2.057 |
| Y645 | P | P-2.167 |

PgIB mutations at positions X, as well as a combination of X were selected for evaluation in larger cultures.

### Conclusions

Shigella4V, Shigella4V Al(OH)₃, Sf2a-, Sf3a-, Sf6-, Ss-EPA all were immunogenic in rabbits and elicited high levels of LPS-specific IgG. Shigella4V and Shigella4V Al(OH)₃ elicited IgG responses against all four O-antigens. Formulation of Shgiella4V with Al(OH)₃ had no significant impact on O-antigen- or EPA-specific IgG responses. There was no statistically significant difference in the levels of O-antigen-specific IgG responses between Shigella4V and monovalent vaccines. No interference due to multivalency was observed. The EPA-specific IgG response is dose dependent

Weak and heterogeneous LPS-specific responses were detected in rabbits injected with formulation buffer only as well as in untreated animals. The responses were significantly lower than in rabbits injected with any of the vaccines tested. Lipid A core specific antibodies may have been elicited by exposure of the rabbits to gram negative bacteria during the course of the study.

The SBA assay showed that elicited response by vaccination of rabbits with the Shigella4V and single serotypes induced a good bactericidal activity against all four serotype Sf2a, Sf3a, Sf6 and Ss. The coadministration of adjuvant with tetravalent vaccine did not show an effect in the generation of antibacterial antibodies. The SBA titers achieved after immunization with monovalent and multivalent formulations with and without adjuvant were comparable (within <2-fold difference) for Shigella flexneri 2a, 3a, 6. The Shigella sonnei monovalent immune sera showed a 4-fold higher SBA titer than the tetravalent vaccine.

### Sequences

SEQ ID NO: 1: Modified detoxified Pseudomonas aeruginosa exotoxin A (EPA) protein carrier used for Sf2E, Sf3E and S16E. The signal peptide (underlined letters) is cleaved off during translocation to the periplasm. The N-glycosylation consensus sites are marked with bold letters. The Leu-Glu to Val mutation (italicized) leads to a significant detoxification of EPA. MW: molecular weight; pl: isoelectric point.

MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRQARGSWSLNWLVPIGHEKPSNIKVFIHELNAGNQLSHMSP
IYTIEMGDELLAKLARDATFFVRAHESNEMQPTLAISHAGVSVVMAQAQP
RREKRWSEWASGKVLCLLDPLDGVYNKDQNATKLAQQRCNLDDTWEGKIY
RVLAGNPAKHDLDIKPTVISHRLHFPEGGSLAALTAHQACHLPLEAFTKD QNATKHRQPRGWEQLEQCGYPVQRLVALYLAARLSWNQVDQVIRNALASP
GSGGDLGEAIREQPEQARLALTLAAAESERFVRQGTGNDEAGAASADVVS
LTCPVAAGECAGPADSGDALLERNYPTGAEFLGDGGDVSFSTRGTQNWTV
ERLLQAHRQLEERGYVFVGYHGTFLEAAQSIVFGGVRARSQDLDAIWRGF
YIAGDPALAYGYAQDQEPDARGRIRNGALLRVYVPRWSLPGFYRTGLTLK DQNATKAPEAAGEVERLIGHPLPLRLDAITGPEEEGGRVTILGWPLAERT
VVIPSAIPTDPRNVGGDLDPSSIPDKEQAISALPDYASQPGKPPREDLK

SEQ ID NO: 2: Modified detoxified Pseudomonas aeruginosa exotoxin A (EPA) protein carrier used for SsE. The signal peptide (underlined letters) is cleaved off during translocation to the periplasm. The O-glycosylation consensus site is bold with the putative O-glycosylated Serine in bold/underlined. The Leu-Glu to Val mutation (italicized) leads to a significant detoxification of EPA. MW: molecular weight; pl: isoelectric point.

MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRQARGSWSLNWLVPIGHEKPSNIKVFIHELNAGNQLSHMSP
IYTIEMGDELLAKLARDATFFVRAHESNEMQPTLAISHAGVSVVMAQAQP
RREKRWSEWASGKVLCLLDPLDGVYNYLAQQRCNLDDTWEGKIYRVLAGN
PAKHDLDIKPTVISHRLHFPEGGSLAALTAHQACHLPLEAFTRHRQPRGW
EQLEQCGYPVQRLVALYLAARLSWNQVDQVIRNALASPGSGGDLGEAIRE
QPEQARLALTLAAAESERFVRQGTGNDEAGAASADVVSLTCPVAAGECAG
PADSGDALLERNYPTGAEFLGDGGDVSFSTRGTQNWTVERLLQAHRQLEE
RGYVFVGYHGTFLEAAQSIVFGGVRARSQDLDAIWRGFYIAGDPALAYGY
AQDQEPDARGRIRNGALLRVYVPRWSLPGFYRTGLTLGTWPKDNTSAGVA SSPTDIKAPEAAGEVERLIGHPLPLRLDAITGPEEEGGRVTILGWPLAER
TVVIPSAIPTDPRNVGGDLDPSSIPDKEQAISALPDYASQPGKPPREDLK

SEQ ID NO: 3 rEPA30 polynucleotide sequence - GlycoTag sequence SEQ ID NO: 20 at N-terminus.

SEQ ID NO:4 rEPA30 amino acid sequence - GlycoTag sequence SEQ ID NO: 20 at N-terminus (DsbA signal sequence and 6xHis Tag (SEQ ID NO: 22) underlined, GlycoTag double underlined).

MKKIWLALAGLVLAFSASASTPLVEAVAASSNAIACKNNAPWYTSSVQSG KYVSAIEPAVKAEEAFDLWNECAKACVLDLKDGVRSSRMSVDPAIADTNG
QGVLHYSMVLEGGNDALKLAIDNALSITSDGLTIRLEGGVEPNKPVRYSY
TRQARGSWSLNWLVPIGHEKPSNIKVFIHELNAGNQLSHMSPIYTIEMGD
ELLAKLARDATFFVRAHESNEMQPTLAISHAGVSVVMAQAQPRREKRWSE
WASGKVLCLLDPLDGVYNYLAQQRCNLDDTWEGKIYRVLAGNPAKHDLDI
KPTVISHRLHFPEGGSLAALTAHQACHLPLEAFTRHRQPRGWEQLEQCGY
PVQRLVALYLAARLSWNQVDQVIRNALASPGSGGDLGEAIREQPEQARLA
LTLAAAESERFVRQGTGNDEAGAASADVVSLTCPVAAGECAGPADSGDAL
LERNYPTGAEFLGDGGDVSFSTRGTQNWTVERLLQAHRQLEERGYVFVGY
HGTFLEAAQSIVFGGVRARSQDLDAIWRGFYIAGDPALAYGYAQDQEPDA
RGRIRNGALLRVYVPRWSLPGFYRTGLTLAAPEAAGEVERLIGHPLPLRL
DAITGPEEEGGRVTILGWPLAERTVVIPSAIPTDPRNVGGDLDPSSIPDK
EQAISALPDYASQPGKPPREDLKHHHHHH

SEQ ID NO: 5 rEPA31 polynucleotide sequence - GlycoTag sequence SEQ ID NO: 19 at N-terminus.

SEQ ID NO: 6 rEPA31_amino acid sequence - GlycoTag sequence SEQ ID NO: 19 at N-terminus (DsbA signal sequence and 6xHis Tag (SEQ ID NO: 22) underlined, GlycoTag double underlined).

MKKIWLALAGLVLAFSASASGAVTEYEADKGVFPTSNASAGVAAAADING KAEEAFDLWNECAKACVLDLKDGVRSSRMSVDPAIADTNGQGVLHYSMVL
EGGNDALKLAIDNALSITSDGLTIRLEGGVEPNKPVRYSYTRQARGSWSL
NWLVPIGHEKPSNIKVFIHELNAGNQLSHMSPIYTIEMGDELLAKLARDA
TFFVRAHESNEMQPTLAISHAGVSVVMAQAQPRREKRWSEWASGKVLCLL
DPLDGVYNYLAQQRCNLDDTWEGKIYRVLAGNPAKHDLDIKPTVISHRLH
FPEGGSLAALTAHQACHLPLEAFTRHRQPRGWEQLEQCGYPVQRLVALYL
AARLSWNQVDQVIRNALASPGSGGDLGEAIREQPEQARLALTLAAAESER
FVRQGTGNDEAGAASADVVSLTCPVAAGECAGPADSGDALLERNYPTGAE
FLGDGGDVSFSTRGTQNWTVERLLQAHRQLEERGYVFVGYHGTFLEAAQS
IVFGGVRARSQDLDAIWRGFYIAGDPALAYGYAQDQEPDARGRIRNGALL
RVYVPRWSLPGFYRTGLTLAAPEAAGEVERLIGHPLPLRLDAITGPEEEG
GRVTILGWPLAERTVVIPSAIPTDPRNVGGDLDPSSIPDKEQAISALPDY
ASQPGKPPREDLKHHHHHH

SEQ ID NO: 7 rEPA32 polynucleotide sequence - GlycoTag sequence SEQ ID NO: 18 in at residue R274.

SEQ ID NO: 8 rEPA32 amino acid sequence - GlycoTag sequence SEQ ID NO: 18 in at residue R274 (DsbA signal sequence and GlycoTag underlined).

MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRQARGSWSLNWLVPIGHEKPSNIKVFIHELNAGNQLSHMSP
IYTIEMGDELLAKLARDATFFVRAHESNEMQPTLAISHAGVSVVMAQAQP
RREKRWSEWASGKVLCLLDPLDGVYNYLAQQRCNLDDTWEGKIYRVLAGN
PAKHDLDIKPTVISHRLHFPEGGSLAALTAHQACHLPLEAFTSAVTGYYL NHGTWPKDNTSAGVASSPTDIKHRQPRGWEQLEQCGYPVQRLVALYLAAR
LSWNQVDQVIRNALASPGSGGDLGEAIREQPEQARLALTLAAAESERFVR
QGTGNDEAGAASADVVSLTCPVAAGECAGPADSGDALLERNYPTGAEFLG
DGGDVSFSTRGTQNWTVERLLQAHRQLEERGYVFVGYHGTFLEAAQSIVF
GGVRARSQDLDAIWRGFYIAGDPALAYGYAQDQEPDARGRIRNGALLRVY
VPRWSLPGFYRTGLTLAAPEAAGEVERLIGHPLPLRLDAITGPEEEGGRV
TILGWPLAERTVVIPSAIPTDPRNVGGDLDPSSIPDKEQAISALPDYASQ
PGKPPREDLK

SEQ ID NO: 9 rEPA33 polynucleotide sequence - GlycoTag sequence SEQ ID NO: 18 in at residue S408.

SEQ ID NO: 10 rEPA33 amino acid sequence - GlycoTag sequence SEQ ID NO: 18 in at residue S408 (DsbA signal sequence and GlycoTag underlined).

MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRQARGSWSLNWLVPIGHEKPSNIKVFIHELNAGNQLSHMSP
IYTIEMGDELLAKLARDATFFVRAHESNEMQPTLAISHAGVSVVMAQAQP
RREKRWSEWASGKVLCLLDPLDGVYNYLAQQRCNLDDTWEGKIYRVLAGN
PAKHDLDIKPTVISHRLHFPEGGSLAALTAHQACHLPLEAFTRHRQPRGW
EQLEQCGYPVQRLVALYLAARLSWNQVDQVIRNALASPGSGGDLGEAIRE
QPEQARLALTLAAAESERFVRQGTGNDEAGAASADVVSLTCPVAAGECAG
PADSGDALLERNYPTGAEFLGDGGDVSAVTGYYLNHGTWPKDNTSAGVAS SPTDIKFSTRGTQNWTVERLLQAHRQLEERGYVFVGYHGTFLEAAQSIVF
GGVRARSQDLDAIWRGFYIAGDPALAYGYAQDQEPDARGRIRNGALLRVY
VPRWSLPGFYRTGLTLAAPEAAGEVERLIGHPLPLRLDAITGPEEEGGRV
TILGWPLAERTVVIPSAIPTDPRNVGGDLDPSSIPDKEQAISALPDYASQ
PGKPPREDLK

SEQ ID NO: 11 rEPA34 polynucleotide sequence - GlycoTag sequence SEQ ID NO: 18 in at residue A519.

SEQ ID NO: 12 Pseudomonas exotoxin A (EPA) amino acid sequence (mature sequence/signal sequence removed). Corresponds to NCBI Reference Sequence WP_016851883.1.

SEQ ID NO: 13 Neisseria meningitidis PilE GlycoTag amino acid sequence (corresponding to residues 55-66 of SEQ ID NO: 21; 12 amino acid long). E.g. Gly Glu Trp Pro Gly Asn Asn Thr Ser Ala Gly Val

SEQ ID NO: 14 Neisseria gonorrhoeae GlycoTag amino acid sequence (corresponding to residues 62-73 of SEQ ID NO: 23; 12 amino acid long). E.g. Gly Thr Trp Pro Lys Asp Asn Thr Ser Ala Gly Val

SEQ ID NO: 15 Neisseria lactamica 020-06 GlycoTag amino acid sequence (corresponding to residues 62-73 of SEQ ID NO: 24; 12 amino acid long). E.g. Gly Thr Phe Pro Ala Gln Asn Ala Ser Ala Gly lle

SEQ ID NO: 16 Neisseria shayeganii 871 GlycoTag amino acid sequence (corresponding to residues 63-74 of SEQ ID NO: 25; 12 amino acids long). E.g. Gly Val Phe Pro Thr Ser Asn Ala Ser Ala Gly Val

SEQ ID NO: 17 Neisseria meningitidis PilE GlycoTag amino acid sequence (corresponding to residues 45-73 of SEQ ID NO: 21; 29 amino acid long). E.g. Ser Ala Val Thr Glu Tyr Tyr Leu Asn His Gly Glu Trp Pro Gly Asn Asn Thr Ser Ala Gly Val Ala Thr Ser Ser Glu lle Lys

SEQ ID NO: 18 Neisseria gonorrhoeae GlycoTag amino acid sequence (corresponding to residues 52-81 of SEQ ID NO: 23; 30 amino acid long). E. g. Ser Ala Val Thr Gly Tyr Tyr Leu Asn His Gly Thr Trp Pro Lys Asp Asn Thr Ser Ala Gly Val Ala Ser Ser Pro Thr Asp lle Lys

SEQ ID NO: 19 Neisseria shayeganii 871 GlycoTag amino acid sequence (corresponding to residues 53-83 of SEQ ID NO: 25; 31 amino acids long). E.g. Gly Ala Val Thr Glu Tyr Glu Ala Asp Lys Gly Val Phe Pro Thr Ser Asn Ala Ser Ala Gly Val Ala Ala Ala Ala Asp lle Asn Gly Lys

SEQ ID NO: 20 Neisseria mucosa ATCC 25996 GlycoTag amino acid sequence (corresponding to residues 52-92 of SEQ ID NO: 26; 41 amino acids long).

SEQ ID NO: 21 Neisseria meningitidis MC58 PilE amino acid sequence (mature sequence; signal sequence removed). Corresponds to NCBI Accession NP_273084.1.

SEQ ID NO: 22 6xHis-tag

SEQ ID NO: 23 Neisseria gonorrhoeae Pilin (NgPilin) amino acid sequence. Corresponds to NCBI GenBank CNT62005.1.

SEQ ID NO: 24 Neisseria lactamica 020-06 Pilin (N/Pilin) amino acid sequence. Corresponds to NCBI GenBank CBN86420.1.

SEQ ID NO:25 Neisseria shayeganii 871 (NsPilin) amino acid sequence. Corresponds to NCBI GenBank EGY51595.1. 100% identity to SEQ ID NOs: 27 and 28.

SEQ ID NO: 26 Neisseria mucosa ATCC 25996 (NmuPilin) amino acid sequence. Corresponds to NCBI GenBank EFC89512.1.

SEQ ID NO: 27 Neisseria shayeganii 871 Pilin amino acid sequence. Corresponds to NCBI GenBank EGY51595.1. 100% identity to SEQ ID NOs: 25 and 28.

SEQ ID NO: 28 Neisseria shayeganii 871 Pilin amino acid sequence. Corresponds to NCBI GenBank EGY51595.1. 100% identity to SEQ ID NOs: 25 and 27.

SEQ ID NO: 29

TWPKDNTSAGVASSPTDIK

SEQ ID NO: 30 EPA sequence from Pseudomonas aeruginosa

AEEAFDLWNECAKACVLDLKDGVRSSRMSVDPAIADTNGQGVLHYSMVLE
GGNDALKLAIDNALSITSDGLTIRLEGGVEPNKPVRYSYTRQARGSWSLN
WLVPIGHEKPSNIKVFIHELNAGNQLSHMSPIYTIEMGDELLAKLARDAT
FFVRAHESNEMQPTLAISHAGVSVVMAQAQPRREKRWSEWASGKVLCLLD
PLDGVYNYLAQQRCNLDDTWEGKIYRVLAGNPAKHDLDIKPTVISHRLHF
PEGGSLAALTAHQACHLPLEAFTRHRQPRGWEQLEQCGYPVQRLVALYLA
ARLSWNQVDQVIRNALASPGSGGDLGEAIREQPEQARLALTLAAAESERF
VRQGTGNDEAGAASADVVSLTCPVAAGECAGPADSGDALLERNYPTGAEF
LGDGGDVSFSTRGTQNWTVERLLQAHRQLEERGYVFVGYHGTFLEAAQSI
VFGGVRARSQDLDAIWRGFYIAGDPALAYGYAQDQEPDARGRIRNGALLR
VYVPRWSLPGFYRTGLTLAAPEAAGEVERLIGHPLPLRLDAITGPEEEGG
RVTILGWPLAERTVVIPSAIPTDPRNVGGDLDPSSIPDKEQAISALPDYA
SQPGKPPREDLK

SEQ ID NO: 31 Consensus sequence (artificial sequence) D/E-X-N-Z-S/T

SEQ ID NO: 32 Consensus sequence (artificial sequence) K-D/E-X-N-Z-S/T-K

SEQ ID NO: 33 Neisseria mucosa PgIL polynucleotide sequence.

SEQ ID NO: 34 Neisseria mucosa PgIL amino acid sequence. Corresponds to NCBI GenBank Accession KGJ31457.1.

SEQ ID NO: 35 Neisseria shayeganii 871 PglL (NsPgIL) amino acid sequence. Corresponds to NCBI GenBank Accession EGY51593.1.

SEQ ID NO: 36 Neisseria sp. 83E34 PgIL polynucleotide sequence.

SEQ ID NO: 37 rEPA1 amino acid sequence - GlycoTag sequence SEQ ID NO: 140 at the N-terminus (DsbA signal sequence underlined, GlycoTag and 6xHis Tag (SEQ ID NO: 22) double underlined)

MKKIWLALAGLVLAFSASASAVTEYYLNHGEWPGNNTSAGVATSSEIKAE
EAFDLWNECAKACVLDLKDGVRSSRMSVDPAIADTNGQGVLHYSMVLEGG
NDALKLAIDNALSITSDGLTIRLEGGVEPNKPVRYSYTRQARGSWSLNWL
VPIGHEKPSNIKVFIHELNAGNQLSHMSPIYTIEMGDELLAKLARDATFF
VRAHESN EMQPTLAISHAGVSVVMAQAQPRREKRWSEWASGKVLCLLDP
LDGVYNYLAQQRCNLDDTWEGKIYRVLAGNPAKHDLDIKPTVISHRLHFP
EGGSLAALTAHQACHLPLEAFTRHRQPRGWEQLEQCGYPVQRLVALYLAA
RLSWNQVDQVIRNALASPGSGGDLGEAIREQPEQARLALTLAAAESERFV
RQGTGNDEAGAASADVVSLTCPVAAGECAGPADSGDALLERNYPTGAEFL
GDGGDVSFSTRGTQNWTVERLLQAHRQLEERGYVFVGYHGTFLEAAQSIV
FGGVRARSQDLDAIWRGFYIAGDPALAYGYAQDQEPDARGRIRNGALLRV
YVPRWSLPGFYRTGLTLAAPEAAGEVERLIGHPLPLRLDAITGPEEEGGR
VTILGWPLAERTVVIPSAIPTDPRNVGGDLDPSSIPDKEQAISALPDYAS
QPGKPPREDLKHHHHHH

SEQ ID NO: 38 rEPA3 amino acid sequence - GlycoTag sequence SEQ ID NO: 20 at C-terminus (DsbA signal sequence and GlycoTag underlined, 6xHis Tag (SEQ ID NO: 22) double underlined)

MKKIWLALAGLVLAFSASAAEEAFDLWNECAKACVLDLKDGVRSSRMSVD
PAIADTNGQGVLHYSMVLEGGNDALKLAIDNALSITSDGLTIRLEGGVEP
NKPVRYSYTRQARGSWSLNWLVPIGHEKPSNIKVFIHELNAGNQLSHMSP
IYTIEMGDELLAKLARDATFFVRAHESNEMQPTLAISHAGVSVVMAQAQP
RREKRWSEWASGKVLCLLDPLDGVYNYLAQQRCNLDDTWEGKIYRVLAGN
PAKHDLDIKPTVISHRLHFPEGGSLAALTAHQACHLPLEAFTRHRQPRGW
EQLEQCGYPVQRLVALYLAARLSWNQVDQVIRNALASPGSGGDLGEAIRE
QPEQARLALTLAAAESERFVRQGTGNDEAGAASADVVSLTCPVAAGECAG
PADSGDALLERNYPTGAEFLGDGGDVSFSTRGTQNWTVERLLQAHRQLEE
RGYVFVGYHGTFLEAAQSIVFGGVRARSQDLDAIWRGFYIAGDPALAYGY
AQDQEPDARGRIRNGALLRVYVPRWSLPGFYRTGLTLAAPEAAGEVERLI
GHPLPLRLDAITGPEEEGGRVTILGWPLAERTVVIPSAIPTDPRNVGGDL
DPSSIPDKEQAISALPDYASQPGKPPREDLKSAVTEYYLNHGEWPGNNTS AGVATSSEIKHHHHHH

### REFERENCES

Kotloff KL et al. (2013) Burden and aetiology of diarrhoeal disease in infants and young children in developing countries (the Global Enteric Multicenter Study, GEMS) a prospective, case-control study. Lancet 382:209-222; Houpt E et al. (2014) Microbiologic methods utilized in the MAL-ED cohort study. Clin Infect Dis 59 Suppl 4:S225-32.

Liu J et al. (2016) Use of quantitative molecular diagnostic methods to identify causes of diarrhoea in children: a reanalysis of the GEMS case-control study. The Lancet 388:1291-1301.

Faridmoayer et al., “Functional Characterization of Bacterial Oligosaccharyltransferases Involved in O-Linked Protein Glycosylation,” J. Bacteriology, pp. 8088-8098, 2007.

Faridmoayer et al., “Extreme Substrate Promiscuity of the Neisseria Oligosaccharyl Transferase Involved in Protein O-Glycosylation,” J. Biol. Chem., vol. 283, no. 50, pp. 34596-34604, 2008.

Tan et al., “Sugar coating: bacterial protein glycosylation and host-microbe interactions,” Trends in Bioch. Sci., vol. 40, no. 7, pp. 342-350, 2015

Pan et al., “Biosynthesis of Conjugate Vaccines Using an O-Linked Glycosylation System,” mBio, vol. 7, no. 2, pp. e00443-16, 2016.

Ihssen et al., “Production of glycoprotein vaccines in Escherichia coli,” Microbial Cell Factories, vol. 9, p. 61, 13 total pages, 2010.

Vik et al., “Broad spectrum O-linked protein glycosylation in the human pathogen Neisseria gonorrhoeae,” PNAS, vol. 106, no. 11, pp. 4447-4452, 2009.

Ravenscroft et al., “Purification and characterization of a Shigella Conjugate Vaccine, Produced by Glycoengineering Escherichia coli,” Glycobiology, vol. 26, no. 1, pp. 51-62, 2016.Glycovaxyn AG, US Pat. No. 8,846,342 (Sep. 30, 2014).

Glycovaxyn AG, U.S. Pat. No. 8,895,014 (Nov. 25, 2014).

ETH Zurich, U.S. Pat. No. 8,753,864 (Jun. 17, 2014).

Tan et al., “Sugar coating: bacterial protein glycosylation and host-microbe interactions,” Trends in Bioch. Sci., vol. 40, no. 7, pp. 342-350, 2015

Wacker et al., “Substrate specificity of bacterial oligosaccharyltransferase suggests a common transfer mechanism for the bacterial and eukaryotic systems,” PNAS, vol. 103, pp. 7088-7093, 2006.

Bentley, “Genetic analysis of the capsular biosynthetic locus from all 90 pneumococcal serotypes,” PLoS Gen, vol. 2, p. e31, 2006.

Borud et al., “Genetic, Structural, and Antigenic Analysis of Glycan Diversity in the O-Linked Protein Glycosylation Systems of Human Neisseria Species,” J. Bacteriology, vol. 192, no. 11, pp. 2816-2829, 2010.

Li et al., “Understanding protein glycosylation pathways in bacteria,” Future Microbiol., vol. 12, no. 1, pp. 59-72, 2017.

Institute of Biotechnology, Academy of Military Medical Sciences, China WO/2016/082597 (Jun. 2, 2016; English language equivalent EP3225690).

Wacker M et al. (2002) N-linked glycosylation in Campylobacter jejuni and its functional transfer into E. coli. Science 298:1790-1793.

Hartley MD et al. (2011) Biochemical characterization of the O-linked glycosylation pathway in Neisseria gonorrhoeae responsible for biosynthesis of protein glycans containing N,N′-diacetylbacillosamine. Biochemistry 50:4936-4948.

Poolman JT, Wacker M (2015) Extraintestinal Pathogenic Escherichia coli, a Common Human Pathogen: Challenges for Vaccine Development and Progress in the Field. J Infect Dis.

Ge et al., “The C-Termainal Domain of AcrA is Essential for the Assembly and Function of the Multidrug Efflux Pump AcrAB-TolC,”J. Bacteriology, vol. 191, no. 13, pp. 4365-4371, 2009.

Glaxosmithkline Biologicals SA WO/2017/067964 (Apr. 27, 2017).

Avci et al., “A mechanism for glycoconjugate vaccine activation of the adaptive immune system and its implications for vaccine design,” Nature Medicine, vol. 17, no. 12, pp. 1602-1610, 2011.

Dagan et al., “Glycoconjugate vaccines and immune interference: a review,” Vaccine, vol. 28, pp. 5513-5523, 2010.

Fisher et al., “Production of Secretory and Extracellular N-Linked Glycoproteins in Escherichia coli,” Applied and Environmental Microbiology, vol. 77, no. 3, pp. 871-881, 2011.

Klein et al., “Design and Characterization of structured Protein Linkers with differing Flexibilities,” Protein Engineering, Design & Selection, vol. 27, no. 10, pp. 325-330, 2014.

Faridmoayer et al., “Extreme Substrate Promiscuity of the Neisseria Oligosaccharyl Transferase Involved in Protein O-Glycosylation,” J. Biol. Chem., vol. 283, no. 50, pp. 34596-34604, 2008.

Bentley, “Genetic analysis of the capsular biosynthetic locus from all 90 pneumococcal serotypes,” PLoS Gen, vol. 2, p. e31, 2006.

University of Alberta, US Pat. No. 9,238,830 (Jan. 19, 2016).

Li et al., “Understanding protein glycosylation pathways in bacteria,” Future Microbiol., vol. 12, no. 1, pp. 59-72, 2017.

Musumeci et al., “In Vitro Activity of Neisseria meningitidis PglL O-Oligosaccharyltransferase with Diverse Synthetic Lipid Donors and a UDP-activated Sugar,” J. Biological Chemistry, vol. 288, no. 15, pp. 10578-10587, 2013.

Ge et al., “The C-Termainal Domain of AcrA is Essential for the Assembly and Function of the Multidrug Efflux Pump AcrAB-TolC,” J. Bacterio Bentley, “Genetic analysis of the capsular biosynthetic locus from all 90 pneumococcal serotypes,” PLoS Gen, vol. 2, p. e31, 2006. logy, vol. 191, no. 13, pp. 4365-4371, 2009.

Pogue et al., “Production of pharmaceutical-grade recombinant aprotinin and a monoclonal antibody product using plant-based transient expression systems,” Plant Biotech. J., vol. 8, pp. 638-654, 2010.

International Application No. PCT/US2007/064857, published as International Publication No. WO2007/109812), imidazoquinoxaline compounds (see International Application No. PCT/US2007/064858, published as International Publication No. WO2007/109813.

Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund’s adjuvant (complete or incomplete). Other adjuvants are oil in water emulsions (such as squalene or peanut oil), optionally in combination with immune stimulants, such as monophosphoryl lipid A (see Stoute et al., N. Engl. J. Med. 336, 86-91 (1997)). Another adjuvant is CpG (Bioworld Today, Nov. 15, 1998.

Bentley, “Genetic analysis of the capsular biosynthetic locus from all 90 pneumococcal serotypes,” PLoS Gen, vol. 2, p. e31, 2006.

Li et al., “Understanding protein glycosylation pathways in bacteria,” Future Microbiol., vol. 12, no. 1, pp. 59-72, 2017.

Institute of Biotechnology, Academy of Military Medical Sciences, China WO/2016/082597 (Jun. 2, 2016; English language equivalent EP3225690).

Aas et al., “Neisseria gonorrhoeae O-linked pilin glycosylation: functional analyses define both the biosynthetic pathway and glycan structure,” Molecular Microbiology, vol. 65, no. 3, pp. 607-624, 2007.

Schulz et al., “Identification of Bacterial Protein O-Oligosaccharyltransferases and their glycoprotein substrates,” PLoS One, vol. 8, no. 5, p. e62768, 2013.

Abeyrathne et al., “Functional Characterization of WaaL, a Ligase Associated with Linking O-Antigen Polysaccharide to the Core of Pseudomonas aeruginosa Lipopolysaccharide,” J. Bacteriol., vol. 187, no. 9, pp. 3002-3012, 2005.

Ruan et al., “The WaaL O-antigen lipopolysaccharide ligase has features in common with metal ion-independent inverting glycosyltransferases,” Glycobiology, vol. 22, no. 2, pp. 288-299, 2012.

Bentley, “Genetic analysis of the capsular biosynthetic locus from all 90 pneumococcal serotypes,” PLoS Gen, vol. 2, p. e31, 2006.

Walker et al., “Molecular cloning, characterization, and complete nucleotide sequence of the gene for pneumolysin, the sulfhydryl-activated toxin of Streptococcus pneumoniae,” Infect. Immun., vol. 55, no. 5, pp. 1184-1189, 1987.

Li et al., “Understanding protein glycosylation pathways in bacteria,” Future Microbiol., vol. 12, no. 1, pp. 59-72, 2017.

Musumeci et al., “In Vitro Activity of Neisseria meningitidis PglL O-Oligosaccharyltransferase with Diverse Synthetic Lipid Donors and a UDP-activated Sugar,” J. Biological Chemistry, vol. 288, no. 15, pp. 10578-10587, 2013.

Borud et al., “Genetic, Structural, and Antigenic Analysis of Glycan Diversity in the O-Linked Protein Glycosylation Systems of Human Neisseria Species,” J. Bacteriology, vol. 192, no. 11, pp. 2816-2829, 2010.

Sun et al., “Design and production of conjugate vaccines against S. Paratyphi A using an O-linked glycosylation system in vivo,” npj Vaccines, vol. 4, pp. 1-9, 2018.

Glycovaxyn AG, U.S. Pat. No. 8,895,014 (Nov. 25, 2014).

Ravenscroft et al., “Purification and characterization of a Shigella Conjugate Vaccine, Produced by Glycoengineering Escherichia coli,” Glycobiology, vol. 26, no. 1, pp. 51-62, 2016.

Glycovaxyn AG, U.S. Pat. No. 8,846,342 (Sep. 30, 2014).

University of Alberta, U.S. Pat. No. 9,238,830 (Jan. 19, 2016).

Perepelov AV et al. (2012) Shigella flexneri O-antigens revisited: final elucidation of the O-acetylation profiles and a survey of the O-antigen structure diversity. FEMS Immunol Med Microbiol 66:201-210.

Kubler-Kielb J, Vinogradov E, Chu C, Schneerson R (2007) O-Acetylation in the O-specific polysaccharide isolated from Shigella flexneri serotype 2a. Carbohydrate research 342:643-647.

Kubler-Kielb Jet al. (2010) Immunochemical studies of Shigella flexneri 2a and 6, and Shigella dysenteriae type 1 O-specific polysaccharide-core fragments and their protein conjugates as vaccine candidates. Carbohydrate research 345:1600-1608.

Hayashi K, Morooka N, Yamamoto Y, et al. Highly accurate genome sequences of Escherichia coli K-12 strains MG1655 and W3110. Mol Syst Biol. 2006;2:2006.0007. doi:10.1038/msb4100049.

Rosano & Ceccarelli, “Recombinant Protein Expression in Escherichia coli: advances and challenges,” Frontiers in Microbiology, vol. 5, no. 172, pp. 1-17, 2014.

Thermo Fisher Scientific, Fusion Protein Purification Resins, 2013.

Sun et al., “Design and production of conjugate vaccines against S. Paratyphi A using an O-linked glycosylation system in vivo,” npj Vaccines, vol. 4, pp. 1-9, 2018.

ETH ZÜRICH WO/2003/074687 (Sep. 12, 2003).

ETH ZÜRICH WO/2006/119987 (Nov. 16, 2006).

University of Alberta, U.S. Pat. No. 9,238,830 (Jan. 19, 2016).

Sun et al., “Design and production of conjugate vaccines against S. Paratyphi A using an O-linked glycosylation system in vivo,” npj Vaccines, vol. 4, pp. 1-9, 2018.

Schulz et al., “Identification of Bacterial Protein O-Oligosaccharyltransferases and their glycoprotein substrates,” PLoS One, vol. 8, no. 5, p. e62768, 2013.

Abeyrathne et al., “Functional Characterization of WaaL, a Ligase Associated with Linking O-Antigen Polysaccharide to the Core of Pseudomonas aeruginosa Lipopolysaccharide,” J. Bacteriol., vol. 187, no. 9, pp. 3002-3012, 2005.

Ruan et al., “The WaaL O-antigen lipopolysaccharide ligase has features in common with metal ion-independent inverting glycosyltransferases,” Glycobiology, vol. 22, no. 2, pp. 288-299, 2012.

Kowarik et al., “N-Linked Glycosylation of Folded Proteins by the Bacterial Oligosaccharyltransferase,” Science, vol. 314, no. 5802, pp. 1148-1150, 2006.

Glycovaxyn AG, U.S. Pat. No. 8,846,342 (Sep. 30, 2014).

University of Alberta, U.S. Pat. No. 9,238,830 (Jan. 19, 2016).

Wacker et al., “N-linked glycosylation in Campylobacter jejuni and its functional transfer into E. coli,” Science, vol. 298, no. 5599, pp. 1790-1793, 2002.

Shepherd et al., “Comparison of O-antigen gene clusters of Escherichia coli (Shigella) sonnei and Plesiomonas shigelloides O17: sonnei gained its current plasmid-borne O-antigen genes from P. shigelloides in a recent event,” Infect. Immun., vol. 68, no. 10, pp. 6065-6061, 2000.

Allured et al., Proc. Natl. Acad. Sci. USA Vol. 83, pp. 1320-1324, March 1986.

Ihssen et al. (2010) Microbial Cell Factories 9:61.

WO 2006/119987

WO 2009/104074

WO2015124769A1

Gray et al (1984) Proc. Nati. Acad. Sci. USA Vol. 81, pp. 2645-2649.

Vasil et al. (1986) Infection and Immunity, May 1986 pages 538 to 548.

Lukac et al. (1988), Infect Immun, 56: 3095-3098, and Ho et al. (2006), Hum Vaccine, 2:89-98.

Feldman et al. 2005, PNAS USA 102:3016-3021.

1-47. (canceled)

48. An immunogenic composition comprising an O-antigen polysaccharide chain from each of S flexneri 2a (Sf2E), S flexneri 3a (Sf3E), S flexneri 6 (Sf6E), and S sonnei (SsE); wherein the O-antigen polysaccharide chains from S. flexneri 2a (Sf2E), S. flexneri 3a (Sf3E), S. flexneri 6 (Sf6E) are separately covalently linked to a protein carrier that has been modified to contain a N- glycosylation consensus sequence.

49. The immunogenic composition of claim 48, wherein the N-glycosylation consensus sequence is D/E-X-N-Z-S/T (SEQ ID NO: 31), wherein X and Z can be any amino acid except proline and optionally wherein PglB is used to transfer the polysaccharide to the N-glycosylation consensus sequence D/E-X-N-Z-S/T (SEQ ID NO: 31), wherein X and Z can be any amino acid except proline.

50. The immunogenic composition of claim 48, wherein SsE is covalently linked to a protein carrier containing an O-glycosylation consensus sequence capable of being glycosylation by PglL, wherein PglL is used to transfer the polysaccharide to the consensus sequence for SsE, TWPKDNTSAGVASSPTDIK (SEQ ID NO: 29).

51. The immunogenic composition of claim 48, wherein the protein carrier is selected from the group consisting of cholera toxin b subunit (CTB), tetanus toxoid (TT), tetanus toxin C fragment (TTc), diphtheria toxoid (DT), CRM 197, Pseudomonas aeruginosa exotoxin A (EPA), C jejuni Acriflavine resistance protein A (CjAcrA), E coli Acriflavine resistance protein A (EcAcrA), and Pseudomonas aeruginosa PcrV (PcrV).

52. The immunogenic composition of claim 48, wherein the protein carrier comprises at least two N-glycosylation consensus sequences.

53. The immunogenic composition of claim 52, wherein the protein carrier is glycosylated at one (Mono-), two (Di-), or at all three N-glycosylation sites (Tri-glycosylated).

54. The immunogenic composition of claim 48, wherein the polysaccharide of Sf2E, Sf3E, and Sf6E are linked covalently via the reducing end of the O-antigen to the side chain nitrogen atom of an asparagine residue; wherein the asparagine residue resides in the D/E-X-N-Z-S/T (SEQ ID NO: 31) N-glycosylation consensus sequence.

55. The immunogenic composition of claim 48, wherein the polysaccharide of SsE is linked covalently via the reducing end of the O-antigen; wherein the glycan has a reducing end structure of
(i) a reducing end structure of Glucose, Galactose. Galactofuranose, Rhamnose. GlcNAc, GalNAc, FucNAc. DATDH, GATDH HexNAc, deoxy HexNAc, diNAcBac, or Pse;
(ii) a reducing end structure of DATDH, GlcNAc, GalNAc, FucNAc, Galactose, or Glucose:
(iii) a reducing end structure of GlcNAc, GalNAc, FucNAc, or Glucose: or
(iv) a S-2 to S-1 reducing end structure of Galactose-β1,4-Glucose; Glucuronic acid-β1, 4-glucose; N-acetyl-fucosamine-α1,3-N-acetyl-galactosamine; Galactose-β1,4-glucose; Rhamnose-β1,4-glucose; Galactofuranose-β1,3-glucose; N-acetyl-altruronic acid-α1,3-4-amino-N-acetyl-fucosamine; or Rhamnose-β1,4-N-acetylgalactosamine.

56. The immunogenic composition of claim 48, wherein the S flexneri 2a, S flexneri 3a, S flexneri 6 antigens are linked via the D-GlcNAc reducing end to the ε-nitrogen atom of an asparagine residue of one of the N-glycosylation consensus sites.

57. A gram-negative host cell which is not S sonnei comprising, the O-antigen polysaccharide chain form S sonnei (SsE).

58. The host cell of claim 57 which is Neisseria, Salmonella, Shigella, Escherichia, Pseudomonas, or Yersinia cell.

59. The host cell of claim 57, comprising a plasmid encoding the carrier protein EPA optionally comprising at least one O-glycosylation consensus sequence suitable for glycosylation by PglL, comprising the amino acid sequence TWPKDNTSAGVASSPTDIK (SEQ ID NO: 29).

60. The host cell of claim 57, comprising a plasmid encoding the oligosaccharyltransferase PglL.

61. A method of producing a tetravalent bioconjugate vaccine, comprising the O-antigen polysaccharide chains from S flexneri 2a (Sf2E), S flexneri 3a (Sf3E), S flexneri 6 (Sf6E), and S sonnei (SsE); comprising the steps of a) culturing four separate host cells (optionally E. coli host cells) engineered to produce bioconjugates under conditions suitable for the production of bioconjugate, b) purifying one bioconjugate selected from the group consisting of Sf2E-EPA, Sf3E-EPA, Sf6E-EPA and SsE-EPA from each culture and c) mixing the Sf2E-EPA, Sf3E-EPA, Sf6EEPA and SsE-EPA bioconjugates, optionally at a ratio of 1:1:1:1.

62. The method of claim 61, wherein Campylobacter jejuni enzyme (PglB) transfers the polysaccharide to a consensus sequence on the carrier protein detoxified Exotoxin A of Pseudomonas aeruginosa (EPA) in E coli for the bioconjugates Sf2E, Sf3E, and Sf6E.

63. The method of claim 61, wherein PglL, transfers the polysaccharide to consensus sequence on the carrier protein detoxified Exotoxin A of Pseudomonas aeruginosa (EPA) in E coli for the bioconjugate SsE.

64. The method of claim 61, wherein the host strain producingSf2E was genetically modified by replacing the polysaccharide biosynthesis (rfb) cluster with S flexneri 2a O-polysaccharide cluster, deletion of the O-antigen ligase waaL, deletion of the araBAD genes required for arabinose metabolism, and replacement of the E coli 016 glycosyltransferase gtrS with S flexneri 2a glycosyltransferase gtrll.

65. The method of claim 61, wherein the host strain of Sf3E is genetically modified by the replacing the polysaccharide biosynthesis (rfb) cluster with S flexneri 3a specific O-polysaccharide cluster, deletion of the O-antigen ligase waaL, deletion of araBAD genes required for arabinose metabolism, and replacement of the E coli 016 glycosyltransferase gtrS with S flexneri 2a glycosyltransferase gtrll.

66. The method of claim 61, wherein the S flexneri 2a glycosyltransferase gtrll is replaced with S flexneri 3a glycosyltransferase gtrX; wherein yeaS gene is replaced with the O-acetyltransferase OAcA gene; wherein yahL gene is replaced with O-acetyltransferase OAcD gene.

67. The method of claim 61, wherein the host strain of SsE was genetically modified by replacing the O16 O-polysaccharide biosynthesis (rfb) cluster with the Plesiomonas shigelloides 017, deletion of the wecA-wzzE, replacing O-antigen waaL with O-oligosaccharyltransferase PgIL, and replacing E coli O16wzz polysaccharide chain length modulator with wzzB polysaccharide chain length modulator of S typhimurium LT2.


SEQUENCE LISTING (38 sequences)

SEQ ID NO: 1 - protein, 649 aa, Artificial Sequence
  source: /note="Description of Artificial Sequence: Synthetic polypeptide"
MKKIWLALAGLVLAFSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNKDQNATKLAQQRCNLDDTWEGKIYRVLAGNPAKHDLDIKPTVISHRLHFPEGGSLAALTAHQACHLPLEAFTKDQNATKHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLKDQNATKAPEAAGEVERLIGHPLPLRLDAITGPEEEGGRVTILGWPLAERTVVIPSAIPTDPRNVGGDLDPSSIPDKEQAISALPDYASQPGKPPREDLK

SEQ ID NO: 2 - protein, 650 aa, Artificial Sequence
  source: /note="Description of Artificial Sequence: Synthetic polypeptide"
MKKIWLALAGLVLAFSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLGTWPKDNTSAGVASSPTDIKAPEAAGEVERLIGHPLPLRLDAITGPEEEGGRVTILGWPLAERTVVIPSAIPTDPRNVGGDLDPSSIPDKEQAISALPDYASQPGKPPREDLK

SEQ ID NO: 3 - skipped

SEQ ID NO: 4 - protein, 679 aa, Artificial Sequence
  source: /note="Description of Artificial Sequence: Synthetic polypeptide"
MKKIWLALAGLVLAFSASASTPLVEAVAASSNAIACKNNAPWYTSSVQSGKYVSAIEPAVKAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLKHHHHHH

SEQ ID NO: 5 - skipped

SEQ ID NO: 6 - protein, 669 aa, Artificial Sequence
  source: /note="Description of Artificial Sequence: Synthetic polypeptide"
MKKIWLALAGLVLAFSASASGAVTEYEADKGVFPTSNASAGVAAAADINGKAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLKHHHHHH

SEQ ID NO: 7 - skipped

SEQ ID NO: 8 - protein, 660 aa, Artificial Sequence
  source: /note="Description of Artificial Sequence: Synthetic polypeptide"
MKKIWLALAGLVLAFSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLEAFTSAVTGYYLNHGTWPKDNTSAGVASSPTDIKHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLK

SEQ ID NO: 9 - skipped

SEQ ID NO: 10 - protein, 660 aa, Artificial Sequence
  source: /note="Description of Artificial Sequence: Synthetic polypeptide"
MKKIWLALAGLVLAFSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSAVTGYYLNHGTWPKDNTSAGVASSPTDIKFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLK

SEQ ID NO: 11 - skipped

SEQ ID NO: 12 - protein, 638 aa, Pseudomonas sp.
MHLIPHWIPLVASLGLLAGGSFASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 13 - protein, 12 aa, Neisseria meningitidis
GEWPGNNTSAGV

SEQ ID NO: 14 - protein, 12 aa, Neisseria gonorrhoeae
GTWPKDNTSAGV

SEQ ID NO: 15 - protein, 12 aa, Neisseria lactamica
GTFPAQNASAGI

SEQ ID NO: 16 - protein, 12 aa, Neisseria shayeganii
GVFPTSNASAGV

SEQ ID NO: 17 - protein, 29 aa, Neisseria meningitidis
SAVTEYYLNHGEWPGNNTSAGVATSSEIK

SEQ ID NO: 18 - protein, 30 aa, Neisseria gonorrhoeae
SAVTGYYLNHGTWPKDNTSAGVASSPTDIK

SEQ ID NO: 19 - protein, 31 aa, Neisseria shayeganii
GAVTEYEADKGVFPTSNASAGVAAAADINGK

SEQ ID NO: 20 - protein, 41 aa, Neisseria mucosa
TPLVEAVAASSNAIACKNNAPWYTSSVQSGKYVSAIEPAVK

SEQ ID NO: 21 - protein, 170 aa, Neisseria meningitidis
MNTLQKGFTLIELMIVIAIVGILAAVALPAYQDYTARAQVSEAILLAEGQKSAVTEYYLNHGEWPGNNTSAGVATSSEIKGKYVKSVEVKNGVVTAQMASSNVNNEIKGKKLSLWAKRQNGSVKWFCGQPVTRDKAKAANDDVTAAAAANGKKIDTKHLPSTCRDASDAS

SEQ ID NO: 22 - protein, 6 aa, Artificial Sequence
  source: /note="Description of Artificial Sequence: Synthetic 6xHis tag"
HHHHHH

SEQ ID NO: 23 - protein, 98 aa, Neisseria gonorrhoeae
MNTLQKGFTLIELMIVIAIVGILAAVALPAYQDYTARAQVSEAILLAEGQKSAVTGYYLNHGTWPKDNTSAGVASSPTDIKGKYVQSVTVATASLPPK

SEQ ID NO: 24 - protein, 148 aa, Neisseria lactamica
MKAIQKGFTLIELMIVIAIVGILAAVALPAYQDYTARAQMSEALTLAEGQKAAVVEYYSDNGTFPAQNASAGIATASAITGKYVAKVEVTGDATSATITSTMKSSGVNNDIKGKTLVLVGKQNSGSFSWECKKGTVDDKFLPSSCRAK

SEQ ID NO: 25 - protein, 143 aa, Neisseria shayeganii
MLKQVQKGFTLIELMIVIAIIGILAAIALPAYQDYIARSQMTEAFNLAGGQKGAVTEYEADKGVFPTSNASAGVAAAADINGKYVEKVEITTDGKIVATMRNSGTSAGIQGKTLTLTPTKQSGSYTWACTSSANSKYLPAACR

SEQ ID NO: 26 - protein, 157 aa, Neisseria mucosa
MKAIQKGFTLIELMIVVAIIGILAAIALPMYGDYTARAQATEGYELLGGMKTPLVEAVAASSNAIACKNNAPWYTSSVQSGKYVSAIEPAVKGETCVLTATFKAASAGVNDKVAEKHIAMTLTPKTGAWSCGTDLDKNVAPAACRDALAAAPANPAQ

SEQ ID NO: 27 - protein, 143 aa, Neisseria shayeganii; identical to SEQ ID NO: 25

SEQ ID NO: 28 - protein, 143 aa, Neisseria shayeganii; identical to SEQ ID NO: 25

SEQ ID NO: 29 - protein, 19 aa, Artificial Sequence
  source: /note="Description of Artificial Sequence: Synthetic peptide"
TWPKDNTSAGVASSPTDIK

SEQ ID NO: 30 - protein, 612 aa, Artificial Sequence
  source: /note="Description of Artificial Sequence: Synthetic polypeptide"
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLK

SEQ ID NO: 31 - protein, 5 aa, Artificial Sequence
  source: /note="Description of Artificial Sequence: Synthetic peptide"
  VARIANT (1)..(1): /replace="Glu"
  MOD_RES (2)..(2): Any amino acid except proline
  MOD_RES (4)..(4): Any amino acid except proline
  VARIANT (5)..(5): /replace="Thr"
  SITE (1)..(5): /note="Variant residues given in the sequence have no preference with respect to those in the annotations for variant positions"
  source: /note="See specification as filed for detailed description of substitutions and preferred embodiments"
DXNXS

SEQ ID NO: 32 - protein, 7 aa, Artificial Sequence
  source: /note="Description of Artificial Sequence: Synthetic peptide"
  VARIANT (2)..(2): /replace="Glu"
  MOD_RES (3)..(3): Any amino acid except proline
  MOD_RES (5)..(5): Any amino acid except proline
  VARIANT (6)..(6): /replace="Thr"
  SITE (1)..(7): /note="Variant residues given in the sequence have no preference with respect to those in the annotations for variant positions"
  source: /note="See specification as filed for detailed description of substitutions and preferred embodiments"
KDXNXSK

SEQ ID NO: 33 - skipped

SEQ ID NO: 34 - protein, 610 aa, Neisseria mucosa
MTYHTLSDGLAESSVKTFWPLWLSFLWICAIPFLSLYRVGPLPSFYLEAISLSGSLVLTLISAHKGLLNIRLPALSIGLFAMAAFWSLQARFMHSTYPGMSDITAWTFVILALGAWSVRGWVTAYGQERVVSIFAWSLLIGATIQAAIVWLQFKGWSNVEWLNGIIARSNGTVNGQLGQRNHLGHYLMWGILAASYLWTARKMSNAAGFLFVLALTATLGLVNSRTILGYIAALFLITPLWFWRTRFQHKRALCIFLLTAILAAAFQFGMGSLLDLFGNVKYETAVERAGTSGFEGSMRQIEWSKAWIAFQSAPWFGHGWNSFAQQTFLINAQQQYFPNNILGVLFTHSHNIVMQLLSEMGIAGTLLATLTLLAAVWRLLGRNQAPASLFLLTAASVSLCHSMLEYPLWYIYFLTVFSLILSLTPSYPKDVSDGPLSIRIRQWAGGIAALSILIGMANLSWEYQNLTQYSRVGKTDDQQTVQNKIDGLQRLSKESPMLAYYADLSLSKRADPTDAYIRPWAEEAALKALTYRPYSSAYQVGLYLYRQGKKEEGAQWMQAMQYYYPYMMYFYADKIRSHPAFAPLLPKLLSDCKDFINAPKHQTAQSCDKP

SEQ ID NO: 35 - protein, 583 aa, Neisseria shayeganii
MMQHLSWPLRSFLFCFAAAYFWGVLSIQHYLPSLGLDMYINQYVAALLSLGLCIFFLGEKNDTCRSISAGVLLWFALLFCLLLQPLLNQIMYPDGLVFPIASVFLCLFLAWGVLNLNETTKQTLVYWVAILLVTVGLLTALSQVAQLMPQNPLTGILFFPISGNRLTGNIAQVNQAAFVEAMGICAAVYLFFNAKEKKIIHFFCAISVFFLAMGVALSASRGGMILAGAAAVSCAIFYAVPLRRRLSLAVLFILLASLGYVLGEFILNRYLPGGVTAVGRFGSGEGFFVLRKALLENAWLAFQDNWLLGIGWGNFNGVNAQNFEEVSWFTSSHHAHNFIAQLAAELGVFGLLIILGFFWVLIRNFKCKLPLYCAFSYSVLSIIFLYSLSEFPLWYLRFLMLAAFFIAVIEKPLTPLSKHYRWLGISISALVFLGSIFYIRHYQNYISVAYQIEMAHDAVEENLDSYKKLGKVFGYGEYDDLMIYTILPVSDENLEHHIQLGKRVLHMNSSPDLLIKQANLLVLAGKSEEADRLYRAVCLSDNLQKRKCVEIFAMLQQAAHDHPDIFTPYFERFKLWYDISLQD

SEQ ID NO: 36 - skipped

SEQ ID NO: 37 - protein, 666 aa, Artificial Sequence
  source: /note="Description of Artificial Sequence: Synthetic polypeptide"
MKKIWLALAGLVLAFSASASAVTEYYLNHGEWPGNNTSAGVATSSEIKAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLKHHHHHH

SEQ ID NO: 38 - protein, 666 aa, Artificial Sequence
  source: /note="Description of Artificial Sequence: Synthetic polypeptide"
MKKIWLALAGLVLAFSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLEAFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRWSLPGFYRTGLTLAAPEAAGEVERLIGHPLPLRLDAITGPEEEGGRVTILGWPLAERTVVIPSAIPTDPRNVGGDLDPSSIPDKEQAISALPDYASQPGKPPREDLKSAVTEYYLNHGEWPGNNTSAGVATSSEIKHHHHHH